(19) United States
(12) Patent Application Publication
(10) Pub. No.: US 2008/0199420 A1
(43) Pub. Date: Aug. 21, 2008

(54) Use Of Polyisobutenyl Succinic Anhydride-Based Block Copolymers In Cosmetic Preparations

(75) Inventors: Volker Wendel, Frankfurt (DE); Darijo Mijolovic, Mannheim (DE)

Correspondence Address: Connolly Bove Lodge & Hutz LLP, 1875 Eye Street, NW, Suite 1100, Washington, DC, 20006, US

(73) Assignee: BASF AKTIENGESELLSCHAFT, Ludwigshafen (DE)

(21) Appl. No.: 11/997,796

(22) PCT Filed: Jul. 31, 2006
(86) PCT No.: PCT/EP06/64855

§ 371 Date: Feb. 4, 2008

(30) Foreign Application Priority Data
Aug. 4, 2005 (EP) 05107216.3

(51) Int. Cl. A61K 8/84; A61Q 99/00

(52) U.S. Cl. 424/78.02

(57) ABSTRACT

The invention relates to novel cosmetic preparations comprising an O/W emulsion, where the O/W emulsion comprises at least one amphiphilic polymer comprising one or more hydrophobic units A and one or more hydrophilic units B, where the hydrophobic units A are formed from polyisobutenes modified with terminal, polar groups, at least one component with an HLB value in the range from 8 to 20, and at least one oil and/or fat phase and water.

The present invention relates to cosmetic preparations comprising an oil-in-water emulsion, where the oil-in-water emulsion comprises at least one amphiphilic polymer comprising one or more hydrophobic units A and one or more hydrophilic units B, where the hydrophobic units A are formed from polyisobutenes modified with terminal, polar groups, at least one component suitable as emulsifier having an HLB value in the range from 8 to 20, at least one oil and/or fat phase, and water.

### PRIOR ART

The prior art discloses derivatives of succinic anhydride substituted by a polyisobutenyl group (PIBSA) in various applications, inter alia as emulsifier and friction-reducing additive in fuels and lubricants. The use of PIBSA and PIBSA derivatives as emulsifier for cosmetic water-in-oil emulsions is likewise known.

WO 04/035635 relates to polymer compositions comprising at least one hydrophobic polymer and at least one modified polyisobutene, to fibers, films, shaped bodies and further processing products thereof constructed from this polymer composition, to a method of producing the polymer composition according to the invention, to a method of producing the fibers, films and shaped bodies constructed from the polymer composition according to the invention, to colored polymer compositions comprising at least one hydrophobic polymer, at least one modified polyisobutene and at least one dye; and to fibers, films and shaped bodies constructed from the colored polymer composition according to the invention and to the use of modified polyisobutenes for treating hydrophobic polymers. The use of such modified polyisobutenes in cosmetics is not described.

WO 93/029309 describes compounds based on polyisobutene and mixtures thereof which are suitable as emulsifiers for oil-in-water emulsions, methods of producing such compounds and the emulsions themselves. Cosmetic preparations comprising oil-in-water emulsions which, in addition to the compounds based on polyisobutene, also comprise emulsifiers with an HLB value in the range from 8 to 20 are not described.

EP-A 1 210 929 describes cosmetic and pharmaceutical compositions comprising at least one emulsifier comprising

a) at least one alkyl chain and/or alkenyl chain having at least 28 carbon atoms obtainable by polymerization of (C₂-C₅)-alkenes, which is linked with

b) at least one carboxylic acid, carboxylic acid derivative, carboxylic acid anhydride, carboxylic acid anhydride derivative, ester and/or amide group.

As emulsifiers, alkenylsuccinic acid anhydrides and derivatives thereof are particularly preferred. Cosmetic preparations comprising oil-in-water emulsions are not described.

WO 02/032382 describes anhydrous pigment pastes comprising a pigment, an anhydrous solvent and a dispersant based on polyisobutenesuccinimide.

EP-A 1 172 089 describes water-in-oil emulsions which comprise, as emulsifier, an oligo- or polyolefin, in particular a polyisobutene with at least 40 carbon atoms and a polar fraction. Further emulsifiers are not used.

U.S. Pat. No. 5,980,922 describes hygiene articles water-in-oil emulsions which comprise, as emulsifiers, for example, polyisobutene derivatives. Oil-in-water emulsions and the use of additional emulsifiers is not described.

DE 197 55 488 A1 describes O/W microemulsions comprising (a) 5 to 30% by weight, preferably 8 to 12% by weight, of oil bodies, (b) 5 to 80% by weight, preferably 15 to 70% by weight, of anionic and/or nonionic emulsifiers and (c) 12 to 30% by weight, preferably 14 to 16% by weight of polyols. The microemulsions are thermally stable and can be produced in a low-temperature process.

The skin is the largest human organ. Among its many functions (for example for temperature regulation and as a sensory organ), the barrier function, which prevents the skin (and thus ultimately the entire organism) from drying out, is probably the most important. At the same time, the skin acts as a protective device against the penetration and the absorption of external substances. This barrier function is effected by the epidermis, which, being the outermost layer, forms the actual protective sheath against the environment. Being about one tenth of the total thickness, it is also the thinnest layer of the skin. The epidermis is a stratified tissue in which the outer layer, the horny layer (Stratum corneum), constitutes the part of importance for the barrier function.

Apart from its barrier effect against external chemical and physical influences, the epidermal lipids also contribute to the holding together of the horny layer and have an effect on the smoothness of the skin. In contrast to the sebaceous gland lipids, which do not form a continuous film on the skin, the epidermal lipids are distributed over the entire horny layer.

The extremely complex interaction of the moisture-binding substances and of the lipids of the upper layers of the skin is very important for the regulation of skin moisture. For this reason, cosmetics generally comprise, besides balanced lipid mixtures and water, water-binding substances.

Besides the chemical composition, however, the physical behavior of these substances is also of importance. The development of very biocompatible emulsifiers and surfactants is therefore desirable. Products formulated therewith aid the liquid-crystalline organization of the intercellular lipids of the Stratum corneum and thus improve the barrier properties of the horny layer. It is particularly advantageous if their molecular constituents consist of substances that are naturally occurring in the epidermis.

Cosmetic skin care is primarily understood as meaning that the natural function of the skin as a barrier against environmental influences (e.g. dirt, chemicals, microorganisms) and against the loss of endogenous substances (e.g. water, natural fats, electrolytes) is strengthened or restored. Impairment of this function can lead to increased absorption of toxic or allergenic substances or to attack by microorganisms and consequently to toxic or allergic skin reactions.

Another aim of skin care is to compensate for the loss by the skin of grease and water caused by daily washing. This is particularly important when the natural regeneration ability is inadequate. Furthermore, skin care products should prevent against environmental influences, in particular against sun and wind, and delay skin aging.

Medicinal topical preparations generally comprise one or more medicaments in an effective concentration. For a clear distinction between cosmetic and medicinal use and corresponding products, reference is made at this point to the legal provisions of the Federal Republic of Germany (e.g. Cosmetics Ordinance, Foods and Drugs Act).

Emulsions

Emulsions are customary cosmetic application forms. Emulsions are generally understood as meaning heterogeneous systems of two liquids that are immiscible or miscible only to a limited extent with one another, which are usually referred to as phases. One of the liquids is in the form of droplets (disperse phase), while the other liquid forms the continuous (coherent) phase.

If the two liquids are water and oil and oil droplets are finely distributed in water, then this is an oil-in-water emulsion (O/W emulsion, e.g. milk). The basic character of an O/W emulsion is determined by the water. If, on the other hand, water droplets are present in fine distribution in oil, then this is a water-in-oil emulsion (W/O emulsion), the basic character of which is determined by the oil.

Less common forms of application are multiple emulsions. These are understood as meaning those emulsions which, in the droplets of the dispersed (or discontinuous) phase, comprise for their part droplets of a further dispersed phase, e.g. W/O/W emulsions or O/W/O emulsions.

In order to be able to ensure the metastability of emulsions, interface-active substances, i.e. emulsifiers, are generally necessary. The droplet diameters of the customary “simple”, i.e. nonmultiple emulsions are in the range from about 1 μm to about 50 μm. Without further coloring additives, such “macroemulsions” are milky-white in color and opaque. Finer “macroemulsions” whose droplet diameters are in the range from about 10⁻¹ μm to about 1 μm are, again without coloring additives, bluish-white in color and opaque. Such “macroemulsions” usually have high viscosity.

Micellar and molecular solutions with particle diameters of less than about 10⁻² μm which, though no longer to be regarded as true emulsions, appear clear and transparent.

Microemulsions are optically isotropic, thermodynamically stable systems which comprise a water-insoluble oil component, emulsifiers and water. The clear or transparent appearance of the microemulsions is a result of the low particle size of the dispersed emulsion droplets. The droplet diameter of microemulsions is in the range from about 10⁻² μm to about 10⁻¹ μm. Microemulsions are translucent and mostly of low viscosity. The viscosity of many microemulsions of the O/W type is comparable with that of water. Microemulsions are often in the literature, although their targeted production is associated with difficulties since the ranges of existence of the microemulsion in the three-phase diagram formed from oil component, water and emulsifiers are in most cases very small and the position of these ranges of existence is greatly influenced to a high degree by structural features of all components and all further ingredients of such systems. On account of their higher stability compared with macroemulsions, finer distribution of the internal phase, the mostly higher effectiveness and the better transdermal penetration of the active ingredients incorporated therein, microemulsions have considerable importance for the formulation of cosmetic and pharmaceutical preparations. A further advantage is that, on account of their low viscosity, they are sprayable. If microemulsions are used as cosmetics, corresponding products are characterized by high cosmetic elegance.

In the field of cosmetic emulsions for skin care and hair care, the consumer sets a large number of requirements. Apart from the cleaning and care effects, which determine the application purpose, importance is placed on such differing parameters as highest possible dermatological compatibility, elegant appearance, optimum sensory impression and storage stability. Some of these features, such as, for example, the skin compatibility, can be determined largely objectively by the person skilled in the art. However, irrespective of these factors, it is known that the finely divided nature of an emulsion is directly connected both to its external appearance and also the storage stability. Consequently, there is great interest in providing emulsions which are characterized by a particularly finely divided nature and, even under thermal stress, do not show a tendency toward agglomeration of the droplets or even toward phase separation. In this connection, reference may be made to the publications by A. Ansmann [Seifen-Öle-Fette-Wachse, 117, 518 (1991)], C. Cabeta [SÖFW Journal, 120, 162 (1994)], P. Hameyer [SÖFW Journal, 121, 216, (1995)] and in particular A. Wadle [Parf.Kosm. 77, 250 (1996)].

The so-called PIT (phase inversion temperature) method has proven particularly advantageous for producing finely divided emulsions. In the single-stage method, the emulsion components are usually initially introduced at room temperature and heated together to about 80° C., during which the lamellar liquid-crystalline phase range is passed through. After cooling to room temperature, a finely divided emulsified oil phase is obtained. In the two-stage hot/hot method, which is preferably used in the industrial sector, the hot, anhydrous phase of oil body and emulsifier is emulsified with some of the water at the same temperature. Here, in the emulsion concentrate, the emulsion passes through a transparent emulsion to which the remaining water is added at about 85° C. As a result of this, the emulsion inverts to give a likewise very finely divided O/W emulsion.

### DISADVANTAGES OF THE PRIOR ART

Cosmetic preparations which are or comprise O/W emulsions and have a high content of pigments often exhibit, besides cosmetically disadvantageous behavior, such as, for example, so-called whitening, i.e. the formation of white marks on the skin, inadequate and unsatisfactory distribution of active ingredients on the application surface.

A further disadvantage of O/W emulsions from the prior art is often their lack of stability at low or high pH values (hydrolysis) and relatively high electrolyte concentrations. For example, this lack of stability can lead to phase separation. Although this can often be remedied to a certain degree through appropriate choice of the emulsifier system, then other disadvantages often arise nevertheless. It is often not possible to dispense with electrolytes since their properties are to be utilized.

Often, the temperatures for producing PIT emulsions are relatively high and reducing the PIT of such emulsions is advantageous.

It was thus an object of the present invention to provide O/W emulsions which are stable toward, in cosmetic terms, high electrolyte concentrations and/or high ionic strengths.

Although the person skilled in the art is already aware of measures with whose help he can in principle arrive at finely divided emulsions, the emulsions of the prior art continue not to be completely satisfactory—on account of the selection of the emulsifiers used. In particular, the external appearance, the sensory impression and the thermal storage resistance are to be improved.

Accordingly, a further object of the present invention was to provide O/W emulsions which, compared with the prior art, are simultaneously characterized by an improved finely divided nature and storage stability, especially at relatively high temperatures.

A further object of the present invention was to provide preparations in the field of care cosmetics, decorative cosmetics and pharmacological galenics with reduced stickiness and/or greasiness.

Furthermore, it was an object of the invention to develop bases for cosmetic preparations which are characterized by good skin compatibility.

It was a further object of the present invention to provide products with as broad an application diversity as possible. Base materials for preparation forms such as cleansing emulsions, face and body care preparations, but also for medicinal-pharmaceutical and/or dermatological application forms should be provided. Examples which may be mentioned are preparations to combat acne and other skin symptoms.

Photoprotective Preparations

The harmful effect of the ultraviolet part of solar radiation on the skin is generally known. While rays with a wavelength of less than 290 nm (UVC region) are absorbed by the ozone layer in the earth's atmosphere, rays in the range between 290 nm and 320 nm (UVB region) cause erythema, simple sunburn or even burns of greater or lesser severity.

The erythema activity maximum of sunlight is generally regarded as the relatively narrow range around 308 nm.

Numerous compounds are known for protecting against UVB radiation; these are mostly derivatives of 3-benzylidenecamphor, of p-aminobenzoic acid, of cinnamic acid, of salicylic acid, of benzophenone and also of 2-phenylbenzimidazole. It is also important to have available filter substances for the range between about 320 nm and about 400 nm (UVA region) since its rays too can also cause damage. Thus, it has been proven that UVA radiation leads to damage of the elastic and collagenous fibers of connective tissue, which makes the skin age prematurely, and that they are to be regarded as a cause of numerous phototoxic and photoallergic reactions. The harmful effect of UVB radiation can be intensified by UVA radiation.

UV radiation also leads to photochemical reactions, the photochemical reaction products interfering in the skin metabolism.

In order to prevent such reactions, the cosmetic or dermatological formulations can additionally comprise antioxidants and/or free-radical scavengers.

The most important inorganic pigments which are known for use in cosmetics as UV absorbers or UV reflectors for protecting the skin against UV rays are the oxides of titanium, zinc, iron, zirconium, silicon, manganese, aluminum, cerium and mixtures thereof.

Compounds which boost the photoprotective effect of a photoprotective agent are referred to as LPF (light protection factor) or SPF (sun protection factor) boosters. It was a further object of the present invention to provide compounds which increase the photoprotective effect of a photoprotective agent and thus act as LPF or SPF boosters.

Deodorants

Preparations based on O/W emulsions are also suitable as bases for deodorants. Cosmetic deodorants serve to eliminate body odor which arises when fresh perspiration, which is in itself odorless, is decomposed by microorganisms. Customary cosmetic deodorants are based on different active principles. In so-called antiperspirants, astringents—primarily aluminum salts such as aluminum hydroxychloride (aluminum chlorohydrate)—reduces the formation of perspiration. By using antimicrobial substances in cosmetic deodorants it is possible to reduce the bacterial flora on the skin. In this connection, ideally, only the odor-causing microorganisms should be effectively reduced. The flow of perspiration itself is not influenced by this, and in an ideal case only microbial decomposition of the perspiration is temporarily stopped. The combination of astringents with antimicrobially effective substances in one and the same composition is also commonplace.

Deodorants should satisfy the following conditions:

1) reliable deodorization;

2) no impairment of the natural biological processes of the skin;

3) harmless nature in the event of incorrect dosage or other use not in accordance with the directions;

4) no accumulation on the skin following repeated application;

5) good ability to be incorporated into customary cosmetic formulations.

Liquid deodorants, for example aerosol sprays, roll-ons and the like, and also solid preparations, for example deodorant sticks, powders, powder sprays, intimate cleansing compositions etc. are known and customary.

It was thus a further object of the present invention to provide preparations which are suitable as a base for cosmetic deodorants or antiperspirants and do not have the disadvantages of the prior art, such as excessively high amounts of emulsifier.

Furthermore, it was an object of the invention to develop cosmetic bases for cosmetic deodorants which are characterized by good skin compatibility.

Furthermore, emulsions for moisturizing the skin, or for stabilizing sensitive active ingredients such as, for example, vitamin C or enzymes, should thus be provided.

In addition, an object of the present invention was to provide hair cosmetic preparations, in particular hair cosmetic preparations for the care of hair and the scalp, which serve in particular to strengthen individual hairs and/or to impart hold and fullness to the hairstyle overall.

Human hair, in particular the cuticle, but also the keratinous region between cuticle and cortex, as the outer sheath of the hair, are exposed to particular stresses as a result of environment influences, as a result of combing and brushing, but also as a result of hair treatment, in particular hair coloring and hair shaping, e.g. permanent waving processes.

If the stress is particularly aggressive, for example bleaching with oxidizing agents such as hydrogen peroxide, in which the pigments distributed in the cortex are oxidatively destroyed, the inside of the hair can also be affected. If human hair is to be colored permanently, in practice only oxidative hair coloring processes are suitable. In oxidatively colored human hair, similarly to bleached hair, microscopic holes can be detected at the points where melanin granules were present. Oxidizing agents react not only with the dye precursors, but also with the hair substance and as a result can cause damage to the hair under certain circumstances. Even washing the hair with aggressive surfactants can stress the hair, at least reduce its appearance or the appearance of the hairstyle overall. For example, certain water-soluble hair constituents (e.g. urea, uric acid, xanthine, keratin, glycogen, citric acid, lactic acid) can be leached out as a result of hair washing.

For these reasons, hair care cosmetics have been used for some time, some of which are intended to be rinsed out of the hair again after they have acted (“rinse off”), and some of which are intended to remain on the hair. The latter can be formulated in such a way that they not only serve to care for the individual hairs, but also improve the appearance of the hairstyle overall, for example by imparting more fullness to the hair, fixing the hairstyle over a longer period or improving its stylability.

Through quaternary ammonium compounds, for example, it is possible to decisively improve the combability of the hair. Such compounds attach to the hair and are often still detectable in the hair after the hair has been washed several times.

There is regularly a need for active ingredients and preparations which care for damaged hair in a satisfactory manner. Preparations which are supposed to give the hairstyle fullness also often prove to be inadequate; they are at least unsuitable to be used as hair care preparations. The hairstyle-fixing preparations of the prior art generally comprise, for example, viscous constituents, which run the risk of giving rise to a feeling of stickiness, which often has to be compensated for by skilful formulation.

Numerous cosmetic preparations are in the form of creams, gels, pastes and generally as application forms which have increased viscosity compared with water. Establishing a desired rheology and in particular a desired viscosity is achieved through the use of rheology modifiers such as, for example, thickeners. Customary cosmetically acceptable thickeners no longer ensure an adequate effect if the electrolyte concentration in the preparations reaches or exceeds certain values.

A further object of the present invention was thus the provision of cosmetically acceptable substances which can also act as thickeners in cosmetic preparations when high electrolyte concentrations are present for which conventional thickeners such as, for example, polyacrylic acids, no longer exhibit the desired effect.

Solution to the Problems

The abovementioned problems are solved through the provision of cosmetic preparations comprising an oil-in-water emulsion, where the oil-in-water emulsion comprises,
a) at least one amphiphilic polymer comprising one or more hydrophobic units A and one or more hydrophilic units B where the hydrophobic units A are formed from polyisobutenes modified with terminal polar groups,
b) at least one component suitable as emulsifier having an HLB value in the range from 8 to 20,
c) at least one oil and/or fat phase and
d) water.

The term amphiphilic is known to the person skilled in the art and indicates that a substance referred to in this way has both lipophilic and hydrophilic properties.

Hydrophobic Units A

In a preferred embodiment of the invention, the hydrophobic units A are obtainable by functionalization of reactive polyisobutene with a number-average molecular weight Mₙ of from 150 to 50 000.

Preference is given to those amphiphilic polymers a) whose hydrophobic units A are formed from a polyisobutene block whose polyisobutene macromolecules have at least 50 mol % terminally arranged double bonds. In a preferred embodiment of the invention, accordingly, at least 50 mol %, preferably at least 60 mol %, of the reactive polyisobutene molecules to be functionalized have terminal double bonds, based on the total number of polyisobutene molecules.

The amphiphilic polymers are generally technical-grade mixtures of substances with a greater or lesser broad molecular weight distribution.

Preferably, each hydrophobic unit A is formed from a polyisobutene block. For the purposes of this invention, polyisobutene is referred to in some places in abbreviated from as PIB.

Polyisobutenes which correspond to the above definition, i.e. which are formed to at least 50 mol % of macromolecules with terminally arranged double bonds, are referred to as so-called reactive polyisobutenes. Here, the term terminally arranged double bonds is understood as meaning either β-olefinic (vinyl) double bonds —[—CH═C(CH₃)₂], or α-olefinic (vinylidene) double bonds —[—C(CH₃)═CH₂]. More preferred reactive polyisobutenes are those in which at least 60 mol %, particularly preferably at least 80 mol %, of the polyisobutene macromolecules, based on the total number of polyisobutene macromolecules, have terminally arranged double bonds.

Suitable reactive polyisobutenes can be obtained, for example, by cationic polymerization of isobutene.

For the synthesis of suitable polyisobutenes, preference is given to using pure isobutene. However, in addition it is also possible to use cationically polymerizable comonomers. However, the amount of comonomers should generally be less than 20% by weight, preferably less than 10% by weight and in particular less than 5% by weight.

Suitable cationically polymerizable comonomers are in particular vinyl aromatics, such as styrene and a-methylstyrene, C₁-C₄-alkylstyrenes, and 2-, 3- and 4-methylstyrene, and 4-tert-butylstyrene, C₃- to C₆-alkenes, such as n-butene, isoolefins having 5 to 10 carbon atoms, such as 2-methylbutene-1,2-methylpentene-1,2-methylhexene-1,2-ethylpentene-1,2-ethylhexene-1 and 2-propylheptene-1.

Suitable isobutene-containing feed materials for the method according to the invention are either isobutene itself or else isobutene-containing C₄-hydrocarbon streams, for example C₄ raffinates, C₄ cuts from the dehydrogenation of isobutane, C₄ cuts from steam crackers or so-called FCC crackers (FCC: Fluid Catalyzed Cracking), provided they are largely freed from 1,3-butadiene present therein. Typically, the concentration of isobutene in C₄-hydrocarbon streams is in the range from 40 to 60% by weight.

Suitable C₄-hydrocarbon streams should generally comprise less than 500 ppm, preferably less than 200 ppm, of 1,3-butadiene. The presence of butene-1, cis- and trans-butene-2 is largely uncritical for the polymerization and does not lead to selectivity losses.

When using C₄-hydrocarbon streams as feed material, the hydrocarbons other than isobutene take on the role of an inert solvent or are copolymerized as comonomer.

Suitable solvents are all organic compounds which are liquid in the selected temperature range of the production of the polyisobutenes and neither cleave off protons nor have free electron pairs.

In particular, mention is to be made of cyclic and acyclic alkanes, such as ethane, iso- and n-propane, n-butane and its isomers, cyclopentane and n-pentane and its isomers, cyclohexane, and n-hexane and its isomers, n-heptane and its isomers, and higher homologs, cyclic and acyclic alkenes, such as ethene, iso- and n-propene, n-butene, cyclopentene, and n-pentene, cyclohexene, and n-hexene, n-heptene, aromatic hydrocarbons, such as benzene, toluene or isomeric xylenes. The hydrocarbons may also be halogenated. Examples of halogenated hydrocarbons include methyl chloride, methyl bromide, methylene chloride, methylene bromide, ethyl chloride, ethyl bromide, 1,2-dichloroethane, 1,1,1-trichloroethane, chloroform or chlorobenzene. It is also possible to use mixtures of the solvents provided no undesired properties arise.

In terms of processing, it is particularly advisable to use solvents which boil in the desired temperature range. The polymerization takes place usually at −80° C. to 0° C., preferably −50° C. to −5° C. and particularly preferably at −30° C. to −15° C.

Pure BF₃, its complexes with electron donors or mixtures thereof can be used as catalyst. Electron donors (Lewis bases) are compounds which have a free electron pair, for example on an O, N, P or S atom, and can form complexes with Lewis acids. This complexation is desired in many cases since, as a result, the activity of the Lewis acid is reduced and secondary reactions are suppressed. Examples of suitable electron donors are ethers, such as diisopropyl ether or tetrahydrofuran, amines such as triethylamine, amides, such as dimethylacetamide, alcohols, such as methanol, ethanol, isopropanol or t-butanol. The alcohols furthermore act as proton source and thus start the polymerization. Protons from ubiquitous traces of water can also activate a cationic polymerization mechanism.

In the cationic polymerization under BF₃ catalysis, largely linear polyisobutenes are obtained which have a particularly high content of α-olefin groups at one chain end. With suitable reaction control, the α-olefin content is not less than 80%.

Reactive polyisobutenes which have reactive α-olefin groups on both chain ends or which are branched can be obtained particularly advantageously through living cationic polymerization. However, linear polyisobutenes which have an α-olefin group only at one chain end can also be synthesized using this method.

In the living cationic polymerization, isobutene is polymerized with a suitable combination of an initiator molecule IXₙ with a Lewis acid S. Details of this method for the polymerization are disclosed, for example, in Kennedy and Ivan, “Carbocationic Macromolecular Engineering”, Hanser Publishers 1992.

Suitable initiator molecules IXₙ have one or more leaving groups X. The leaving group X is a Lewis base, which can also be yet further substituted. Examples of suitable leaving groups comprise the halogens fluorine chlorine, bromine and iodine, straight-chain and branched alkoxy groups, such as C₂H₅O—, n-C₃H₇O—, i-C₃H₇O—, n-C₄H₉O—, i-C₄H₉0-, sec-C₄H₉0- or t-C₄H₉0-, and straight-chain and branched carboxy groups such as CH₃ CO—O—, C₂H₅ CO—O—, n-C₃H₇ CO—O—, i-C₃H₇ CO—O—, n-C₄H₉ CO—O—, i-C₄H₉ CO—O—, sec-C₄H₉ CO—O—, t-C₄H₉ CO—O—. Joined to the leaving group or groups is the molecular moiety I, which, under reaction conditions, can form sufficiently stable carbocations I⁺. To trigger the polymerization, the leaving group is abstracted using a suitable Lewis acid S: I−X+S→I⁺+XS⁻ (shown here only for the case n=1). The resulting carbocation I⁺ starts the cationic polymerization and is incorporated into the resulting polymer. Suitable Lewis acids S are, for example, AlY₃, TiY₄, BY₃, SnY₄, ZnY₂, where Y is fluorine, chlorine, bromine or iodine. The polymerization reaction can be terminated by destroying the Lewis acid, for example by its reaction with alcohol. This process forms polyisobutene which has terminal —C(CH₃)₂-Z groups, which can then be converted into α- and β-olefin end groups.

As initiator molecule, preference is given to structures which can form tertiary carbocations. Particular preference is given to radicals derived from the lower oligomers of isobutene H—[CH₂—C(CH₃)₂]ₙ—X, where n is preferably 2 to 5. Linear reactive polyisobutenes formed with such initiator molecules have a reactive group only at one end.

Linear polyisobutenes which have reactive groups at both ends can be obtained using initiator molecules IXQ which have two leaving groups X and Q, where X and Q may be identical or different. In practice, compounds which comprise —C(CH₃)₂—X groups have proven useful. Examples comprise straight-chain or branched alkylene radicals CₙH₂ₙ (where n can preferably assume values from 4 to 30), which can also be interrupted by a double bond or an aromatic, such as

X—(CH₃)₂C—CH₂—C(CH₃)₂-Q, X—(CH₃)₂C—CH₂—C(CH₃)₂CH₂—C(CH₃)₂-Q,

X—(CH₃)₂C—CH₂—C(CH₃)₂CH₂—C(CH₃)₂CH₂—C(CH₃)₂-Q or

X—(CH₃)₂C—CH₂—C(CH₃)₂CH₂—C(CH₃)₂CH₂—C(CH₃)₂—CH₂—C(CH₃)₂-Q,

X—(CH₃)₂C—CH═CH—C(CH₃)₂-Q or para and/or meta

X—(CH₃)₂C—C₆H₄—C(CH₃)₂-Q.

Branched polyisobutenes can be obtained by using initiator molecules IXₙ which have 3 or more leaving groups, where the leaving groups may be identical or different.

Examples of suitable initiator molecules comprise X—(CH₃)₂C—C₆H₃—[C(CH₃)₂-Q]-C(CH₃)₂—P as 1,2,4- and/or 1,3,5-isomer, where the leaving groups are preferably identical, but may also be different. Further examples of mono-, di-, tri- or polyfunctional initiator molecules can be found in the work by Kennedy and Ivan cited at the start, and the literature cited therein.

Suitable polyisobutenes which have a large number of α-olefin groups in the vicinity of one and/or at one chain end are, for example, the Glissopal® grades from BASF Aktiengesellschaft, for example Glissopal®550, 1000, 1300 or 2300, and the Oppanol® grades from BASF AG, such as Oppanol®B10 or B12.

Of particular suitability for the cosmetic preparations according to the invention are those polymers a) which have a polyisobutene block with a number-average molecular weight Mₙ in the range from 150 to 50 000 g/mol, preferably in the range from 200 to 20 000 g/mol and particularly preferably in the range from 450 to 5000 g/mol.

Depending on the polymerization method, the polydispersity index (PDI), i.e. the ratio of weight-average and number-average molecular weight, of the polyisobutenes which can be used preferably is in the range from 1.05 to 10, preferably in the range from 1.05 to 5, particularly preferably in the range from 1.05 to 2.0. The method of determining the polydispersity (PDI) and the number-average and weight-average molecular weight is described, for example, in the Analytiker-Taschenbuch, Volume 4, pages 433 to 442, Berlin 1984.

Suitable amphiphilic block copolymers a) for the use in the preparations according to the invention are block copolymers consisting of at least one hydrophobic unit A formed from reactive polyisobutenes with at least one polar functional group as anchor group and at least one hydrophilic unit B formed from a polyalkylene oxide or a polyethyleneimine.

To introduce the hydrophilic unit B, the reactive polyisobutenes are functionalized by introducing polar groups. Depending on the type of polar group(s), the functionalized polyisobutenes are reacted either with alkylene oxides, such as, for example, ethylene oxide or propylene oxide, or in a polymer-analogous reaction with polyalkylene oxides, such as, for example, polyethylene oxide, polypropylene oxide or ethylene oxide-propylene oxide copolymers or polyethyleneimines.

If the amphiphilic block copolymers a) are prepared by reacting one or more functionalized polyisobutenes with alkylene oxides, then the hydrophilic block of the described block copolymer is only formed during the reaction.

By contrast, with the specified polymer-analogous reactions of one or more functionalized polyisobutenes with polyalkylene oxides or polyethyleneimines, preformed hydrophilic blocks B are used.

Preferably, the amphiphilic block copolymers a) are produced in a polymer-analogous reaction of hydrophobic unit A, formed from reactive polyisobutene with at least one functional group, with at least one hydrophilic unit B, formed from a polyalkylene oxide.

In principle, the invention is not restricted with regard to the one or more hydrophilic units B that can be used to form the amphiphilic polymers a).

Units B which are readily soluble in water and sparingly soluble in oil are particularly advantageous.

In order to join the hydrophilic units B with the hydrophobic units, the reactive polyisobutenes are functionalized with the introduction of polar groups. The degree of functionalization of the modified polyisobutene derivatives with terminal, polar groups is at least 50%, preferably at least 60% and very particularly preferably at least 80%. In the case of the polymers having polar groups only at one chain end, this information refers only to this one chain end.

In the case of the polyisobutenes having polar groups at both chain ends, and also the branched products, the information concerning the degree of functionalization refers to the total number of all chain ends. The nonfunctionalized chain ends are either those which have no reactive group at all or those which do have a reactive group, but were not converted in the course of the functionalization reaction.

The term “polar group” is known to the person skilled in the art. The polar groups may either be protic or aprotic polar groups. The modified polyisobutenes thus have a hydrophobic molecular moiety of a polyisobutene radical, and a molecular moiety, which has at least a certain hydrophilic character, of terminal, polar groups. These are preferably strongly hydrophilic groups. The terms “hydrophilic” and “hydrophobic” are known to the person skilled in the art.

Suitable reactions for introducing polar groups (functionalization) are known in principle to the person skilled in the art.

In principle, the functionalization of the polyisobutenes used according to the invention can be carried out in one or more stages.

In a preferred embodiment, the functionalization of the polyisobutene used according to the invention takes place in one or more stages and is selected from:
i) reaction of the reactive polyisobutene with aromatic hydroxy compounds in the presence of an alkylation catalyst to give aromatic hydroxy compounds alkylated with polyisobutenes,
ii) reaction of the reactive polyisobutene with a peroxy compound to give an epoxidized polyisobutene,
iii) reaction of the reactive polyisobutene with an alkene which has a double bond substituted by electron-attracting groups (enophile), in an ene reaction,
iv) reaction of the reactive polyisobutene with carbon monoxide and hydrogen in the presence of a hydroformylation catalyst to give a hydroformylated polyisobutene,
v) reaction of the reactive polyisobutene with a phosphorus halide or a phosphorus oxychloride to give a polyisobutene functionalized with phosphono groups,
vi) reaction of the reactive polyisobutene with a borane and subsequent oxidative cleavage to give a hydroxylated polyisobutene,
vii) reaction of the reactive polyisobutene with an SO₃ source, preferably acetyl sulfate or oleum, to give a polyisobutene with terminal sulfo groups,
viii) reaction of the reactive polyisobutene with oxides of nitrogen and subsequent hydrogenation to give a polyisobutene with terminal amino groups,
ix) reaction of the reactive polyisobutene with hydrogen sulfide or a thiol to give a polyisobutene functionalized with thiol groups.

Particular preference is given to the embodiments iii) and vi) and very particular preference to the embodiment iii).

The abovementioned reactions i) to ix) are described in detail in WO 04/035635, p. 12, 1.26 to p. 27, 1.2. Reference is made here to this description in its entirety.

Re i) alkylation of aromatic hydroxy compounds

For the functionalization, the reactive polyisobutene can be reacted with an aromatic hydroxy compound in the presence of an alkylation catalyst. Suitable catalysts and reaction conditions of this so-called Friedel-Crafts alkylation are described, for example, in J. March, Advanced Organic Chemistry, 4th edition, Verlag John Wiley & Sons, pp. 534-539, to which reference is hereby made.

The aromatic hydroxy compound used for the alkylation is preferably selected from phenolic compounds having 1, 2 or 3 OH groups, which, if appropriate, may have at least one further substituent. Preferred further substituents are C₁-C₈-alkyl groups and in particular methyl and ethyl. Preference is given in particular to compounds of the general formula,

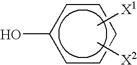

in which X¹ and X², independently of one another, are hydrogen, OH or CH₃. Particular preference is given to phenol, the cresol isomers, catechol, resorcinol, pyrogallol, fluoroglucinol and the xylenol isomers. In particular, phenol, o-cresol and p-cresol are used. If desired, mixtures of the abovementioned compounds can also be used for the alkylation.

The catalyst is preferably selected from Lewis-acidic alkylation catalysts, which, for the purposes of the present application, are understood as meaning both individual acceptor atoms and also acceptor-ligand complexes, molecules, etc., provided they have overall (outwardly) Lewis-acidic (electron acceptor) properties. These include, for example, AlCl₃, AlBr₃, BF₃, BF₃2 C₆H₅OH, BF₃[O(C₂H₅)₂]₂, TiCl₄, SnCl₄, AlC₂H₅Cl₂, FeCl₃, SbCl₅ and SbF₅. These alkylation catalysts can be used together with a cocatalyst, for example an ether. Suitable ethers are di(C₁-C₈)alkyl ethers, such as dimethyl ether, diethyl ether, di-n-propyl ether, and tetrahydrofuran, di(C₅-C₈)cycloalkyl ethers, such as dicyclohexyl ether and ethers with at least one aromatic hydrocarbon radical, such as anisole. If, for the Friedel-Crafts alkylation, a catalyst-cocatalyst complex is used, then the molar quantitative ratio of catalyst to cocatalyst is preferably in a range from 1:10 to 10:1. The reaction can also be catalyzed with protic acids, such as sulfuric acid, phosphoric acid, trifluoromethanesulfonic acid. Organic protic acids can also be in polymer-bound form, for example as ion exchanger resin.

The alkylation can be carried out solvent-free or in a solvent. Suitable solvents are, for example, n-alkanes and mixtures thereof and alkyl aromatics, such as toluene, ethylbenzene and xylene, and halogenated modifications thereof.

The alkylation is preferably carried out at temperatures between −10° C. and +100° C. The reaction is usually carried out at atmospheric pressure, but can also be carried out at higher or lower pressures.

Through appropriate choice of the molar quantitative ratios of aromatic hydroxy compound to polyisobutene and of the catalyst it is possible to establish the achieved fraction of alkylated products and their degree of alkylation. Thus, for example, essentially monoalkylated polyisobutenylphenols are generally obtained with an excess of phenol or in the presence of a Lewis-acidic alkylation catalyst if additionally an ether is used as cocatalyst.

The reaction of polyisobutenes with phenols in the presence of suitable alkylation catalysts is disclosed, for example, in U.S. Pat. No. 5,300,701 and WO 02/26840.

For the further functionalization, a polyisobutenylphenol obtained in step i) can be subjected to a reaction in the sense of a Mannich reaction with at least one aldehyde, for example formaldehyde, and at least one amine which has at least one primary or secondary amine function, giving a polyisobutene-alkylated and additionally at least partially aminoalkylated compound. It is also possible to use reaction and/or condensation products of aldehyde and/or amine. The preparation of such compounds is described in WO 01/25 293 and WO 01/25 294, to which reference is hereby made in their entirety.

In a further embodiment, for the further functionalization, a polyisobutenylphenol obtained in step i) can be subjected to a hydrogenation step. The preparation of such compounds is described in the unpublished German patent application No. 102005021093.7, to which reference is hereby made in its entirety.

For the preparation of the described amphiphilic block copolymers a), in a further step, a polyisobutenylphenol obtained in step i), which has, if appropriate, been subjected to a Mannich reaction or hydrogenation, is reacted with alkylene oxides. In this reaction, one or more hydrophilic unit(s) B of polymer a) are formed by graft polymerization on the terminally functionalized polyisobutene A. The number of hydrophilic units B is governed here by the number of OH groups of the polyisobutenephenol obtained in step i). If, for example, phenol is used for the functionalization, a polymer a) with A-B structure is obtained.

Alkylene oxides which can be used are preferably ethylene oxide or ethylene oxide/propylene oxide mixtures, preferably with a fraction of from 0 to 50% by weight of propylene oxide, particularly preferably with a fraction of from 0 to 20% by weight of propylene oxide, very particularly preferably of ethylene oxide. Here, the alkylene oxide block which forms is a random copolymer, a gradient copolymer, an alternating or a block copolymer of ethylene oxide and propylene oxide. Besides ethylene oxide and propylene oxide, the following pure alkylene oxides or else mixtures can be used: 1,2-butene oxide, 2,3-butene oxide, 2-methyl-1,2-propene oxide(isobutene oxide), 1,2-pentene oxide, 2,3-pentene oxide, 2-methyl-1,2-butene oxide, 3-methyl-1,2-butene oxide, 2,3-hexene oxide, 3,4-hexene oxide, 2-methyl-1,2-pentene oxide, 2-ethyl-1,2-butene oxide, 3-methyl-1,2-pentene oxide, decene oxide, 4-methyl-1,2-pentene oxide, styrene oxide or can be formed from a mixture of oxides of industrially available raffinate streams.

In a further embodiment, the resulting polyisobutenylphenols which have, if appropriate, been subjected to a Mannich reaction or hydrogenation are reacted with, for example, phosphorus oxychloride to give a phosphoric half-ester. This is reacted in a subsequent step with polyethyleneimines, alkylene oxides or polyalkylene oxides to give the described block copolymers a).

If it is a reaction with alkylene oxides, then one or more hydrophilic unit(s) B of polymer a) are produced by graft polymerization onto the polyisobutene A terminally functionalized with phosphoric half-ester groups. The number of hydrophilic units B depends on the number of OH groups of the resulting phosphated polyisobutenephenol. If, for example, phenol is used for the functionalization of polyisobutene and reacted with phosphorus oxychloride, a hydrophobic unit A with two OH groups is obtained which forms the amphiphilic polymer a) with A-B₂ structure by means of alkoxylation. If PIB phenol derivatives which have been further reacted subsequently in a Mannich reaction and still comprise free N—H groups after the reaction are subjected to an alkoxylation, then, besides the OH groups of the phosphoric half-ester group, these N—H groups can also react with alkylene oxides and thus form a further hydrophilic unit B.

If the polyisobutenephenols reacted with, for example, phosphorus oxychloride which have, if appropriate, been subjected to a Mannich reaction or hydrogenation are reacted with polyethyleneimines or polyalkylene oxides, then these are polymer-analogous reactions with a preformed hydrophilic unit B. The polyalkylene oxides used must comprise at least one reactive group selected from the group consisting of OH, SH, NH₂ or NH.

Preferably, for the formation of amphiphilic polymers a) of polyisobutene A functionalized with phosphoric half-ester, use is made of polyalkylene oxides with at least one OH group.

In a further embodiment, the resulting polyisobutenylphenols which have, if appropriate, been subjected to a Mannich reaction or hydrogenation are reacted with, for example, sulfuric acid or oleum to give a sulfuric half-ester. This is reacted in a subsequent step with polyethyleneimines, alkylene oxides or polyalkylene oxides to give the described block copolymers a).

As already described for the phosphoric half-esters, the reaction of sulfuric half-esters with alkylene oxides is a graft polymerization. The number of hydrophilic units B depends here on the number of OH groups of the resulting sulfated polyisobutenephenol. If, for example, phenol is used for the functionalization of PIB and reacted with oleum, a hydrophobic unit A with an OH group is obtained which forms the polymer a) with. A-B structure by means of alkoxylation. If PIB phenol derivatives which have been further reacted subsequently in a Mannich reaction and still comprise free N—H groups after the reaction are subjected to an alkoxylation, then, besides the OH groups of the sulfuric half-ester group, these N—H groups can also enter into a graft polymerization with alkylene oxides and thus form a further hydrophilic unit B.

If sulfated polyisobutenephenols which have been subjected beforehand, if appropriate, to a Mannich reaction or hydrogenation are reacted with polyethyleneimines or polyalkylene oxides, then these are polymer-analogous reactions with a preformed hydrophilic unit B. The polyalkylene oxides used must have at least one group selected from OH, SH, NH₂ or NH.

Preferably, for the formation of amphiphilic polymers a) of polyisobutene A functionalized with sulfuric acid half-ester, use is made of polyalkylene oxides with at least one OH group. Which polyalkylene oxides are preferably used is described in the section “Hydrophilic units B”.

Re ii) Epoxidation

For the functionalization, the reactive polyisobutene can be reacted with at least one peroxy compound to give an epoxidized polyisobutene. Suitable methods for the epoxidation are described in J. March, Advanced Organic Chemistry, 4th edition, Verlag John Wiley & Sons, pp. 826-829, to which reference is hereby made. As peroxy compound, preference is given to using at least one peracid, such as m-chloroperbenzoic acid, performic acid, peracetic acid, trifluoroperacetic acid, perbenzoic acid and 3,5-dinitroperbenzoic acid. The production of the peracids can take place in situ from the corresponding acids and H₂O₂, if appropriate in the presence of mineral acids. Further suitable epoxidation reagents are, for example, alkaline hydrogen peroxide, molecular oxygen and alkyl peroxides, such as tert-butyl hydroperoxide. Suitable solvents for the epoxidation are, for example, customary, nonpolar solvents. Particularly suitable solvents are hydrocarbons, such as toluene, xylene, hexane or heptane.

For the further functionalization, the epoxidized polyisobutenes which are obtained in step ii) can be reacted with ammonia, giving polyisobutene amino alcohols (EP-A 0 476 785).

For the preparation of the described block copolymers a), in a further step, the resulting epoxidized polyisobutenes are reacted with alkylene oxides. The reaction is a graft polymerization in which the hydrophilic units B are formed during the reaction. The number of hydrophilic units B depends on the number of epoxide groups per molecule of the polyisobutene epoxide. Which alkylene oxides are preferably used is described in the section “Hydrophilic units B”.

Re iii) Ene Reaction

For the functionalization, the reactive polyisobutene can furthermore be reacted with at least one alkene which has a low-electron double bond in an ene reaction (see, for example, DE-A 195 19 042, DE-A 4 319 671, DE-A 4 319 672 or H. Mach and P. Rath in “Lubrication Science II (1999), pp. 175-185, to the entire contents of which reference is made). In the ene reaction, an alkene, referred to as ene, having an allyl-position hydrogen atom is reacted with a low-electron alkene, the so-called enophile, in a pericyclic reaction, comprising a carbon-carbon bond linkage, a double bond shift and a hydrogen transfer. Presently, the reactive polyisobutene reacts as ene. Suitable enophiles are compounds as are also used as dienophiles in the Diels-Alder reaction. Suitable enophiles are fumaryl dichloride, fumaric acid, maleoyl dichloride, maleic anhydride and maleic acid, preferably maleic anhydride and maleic acid. In the process, the succinic acid derivatives of the general formula Ia, Ib or Ic are formed in which X³ is a polyisobutene group with a number-average molecular weight Mₙ of from 150 to 50 000, preferably 200 to 20 000, particularly preferably from 450 to 5000.

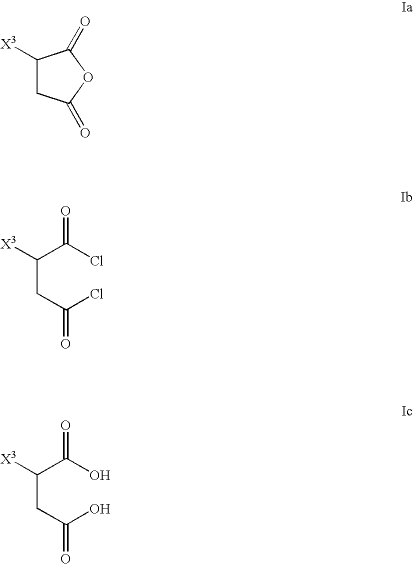

As enophile, very particular preference is given to using maleic anhydride (formula Ia). The process produces polyisobutenes functionalized with succinic anhydride groups (polyisobutenylsuccinic anhydride, PIBSA), as disclosed in EP-A 0 156 310.

The ene reaction can, if appropriate, be carried out in the presence of a Lewis acid as catalyst. For example, aluminum chloride and ethylaluminum chloride are suitable.

In the reaction, a new α-olefin group is produced at the chain end. For the further functionalization and production of the described block copolymers, the polyisobutene derivatized with succinic anhydride groups is subjected to a subsequent reaction which is selected from:

α) graft polymerization with at least one abovementioned alkylene oxide to give a polyisobutene functionalized with two succinic ester groups (per succinic anhydride group),

β) hydrolysis to give a polyisobutene functionalized with succinic acid groups, where the succinic acid groups are reacted as under a) with alkylene oxides by means of graft polymerization,

χ) reaction with maleic anhydride to give a product with two succinic anhydride groups at the chain end (so-called PIBBSA), where the reaction is as under a) with alkylene oxides by means of graft polymerization,

δ) reaction with at least one amine to give a polyisobutene functionalized at least partially with succinimide groups and/or succinamide groups, which is reacted in a further reaction with said alkylene oxides by means of graft polymerization,

ε) reaction with at least one alcohol or thioalcohol to give a polyisobutene functionalized with succinic ester groups or succinic thioester groups, which is reacted in a further reaction with said alkylene oxides by means of graft polymerization,

φ) reaction with at least one polyethyleneimine to give a polyisobutene functionalized at least partially with succinimide groups and/or succinamide groups,

γ) reaction with at least one polyalkylene oxide which has at least one hydroxy group to give a polyisobutene functionalized at least partially with succinic ester groups,

η) reaction with at least one polyalkylene oxide which has at least one amino group to give a polyisobutene functionalized at least partially with succinimide groups and/or succinamide groups,

τ) reaction with at least one polyalkylene oxide which has at least one thiol group to give a polyisobutene functionalized at least partially with succinic thioester groups,

φ) if, after the reaction of the succinic anhydride group, free carboxyl groups are still present, these can also be converted to salts. Suitable preferred cations in salts are primarily alkali metal cations, ammonium ions, and alkylammonium ions.

Re χ)

The polyisobutenes derivatized with one succinic anhydride group per chain end can be reacted in an exhaustive ene reaction with an excess of maleic anhydride to give polyisobutenes functionalized with in part two succinic anhydride groups per chain end. The polyisobutenes functionalized in this way can be reacted with alkylene oxides by means of graft polymerization, where in each case two succinic ester groups are formed per anhydride group.

Re δ) and ε)

For the further functionalization, the succinic anhydride groups can be reacted, for example, with polar reactants, such as alcohols, thioalcohols or amines. Suitable polar reactants are preferably alcohols ROH, thioalcohols RSH or primary amines RNH₂ or secondary amines RR′NH, where R is a linear or branched saturated hydrocarbon radical which carries at least two substituents selected from the group OH, SH, NH₂ or NH₃⁺ and, if appropriate, one or more CH(O) groups and, if appropriate, has nonadjacent —O— and/or —NH— and/or tertiary —N— groups, and R′, independently R, has the same meaning. Here, it is possible for both carboxylic acid groups of the succinic anhydride to react or else only one, while the other carboxylic acid group is present as free acid group or as salt. In a further reaction, the free substituents (substituents not reacted with anhydride) are modified by alkoxylation, giving the described block copolymers a).

Re φ)

For the production of the described block copolymers a), the succinic anhydride groups can be reacted with polyethyleneimines in a polymer-analogous way, where one or more polyisobutene chains are joined per polyethyleneimine chain, depending on the reaction procedure. The binding takes place via succinimide groups and/or succinamide groups. The polyethyleneimines are preformed hydrophilic units B.

Re γ), η) and τ)

For the production of the described block copolymers a), the succinic anhydride groups are reacted with polyalkylene oxides in a polymer-analogous manner. In this connection, the polyalkylene oxides used must have at least one group selected from OH, SH, NH₂ or NH. The polyethylene oxides are preformed hydrophilic units B.

Which alkylene oxides and polyalkylene oxides are preferably used is described in the section “Hydrophilic units B”.

Further synthesis variants for the derivatization of succinic anhydride groups are given in DE-A-101 25 158. It is also known to the person skilled in the art to convert a succinic anhydride group into a succinimide group under suitable conditions.

In a further embodiment, reactive polyisobutene can be free-radically copolymerized with maleic anhydride (cf. WO 95/07944, WO 01/55059, WO 90/03359). The strictly alternating copolymers obtained in this way can be further reacted as described above. Preference is given to the reactions with alkylene oxides, polyalkylene oxides or polyethyleneimines. Which alkylene oxides or polyalkylene oxides are preferably used is described in the section “Hydrophilic units B”.

Re iv) Hydroformylation

For the functionalization, the reactive polyisobutene can be subjected to a reaction with carbon monoxide and hydrogen in the presence of a hydroformylation catalyst, giving a hydroformylated polyisobutene.

Suitable catalysts for the hydroformylation are known and comprise preferably a compound or a complex of an element of subgroup VIII of the Periodic Table of the Elements, such as Co, Rh, Ir, Ru, Pd or Pt. For influencing the activity and/or selectivity, preference is given to using hydroformylation catalysts modified with N— or P-containing ligands. Suitable salts of these metals are, for example, the hydrides, halides, nitrates, sulfates, oxides, sulfides or the salts with alkyl- or arylcarboxylic acids or alkyl- or arylsulfonic acids. Suitable complex compounds have ligands which are selected, for example, from halides, amines, carboxylates, acetyl acetonate, aryl- or alkylsulfonates, hydride, CO, olefins, dienes, cycloolefins, nitriles, N-containing heterocycles, aromatics and heteroaromatics, ethers, PF₃, phospholene, phosphabenzenes, and mono-, di- and polydentate phosphine, phosphinite, phosphonite, phosphoramidite and phosphite ligands.

In general under hydroformylation conditions, the catalysts or catalyst precursors used in each case form catalytically active species of the general formula HₓM_{y}(CO)_{z}L_{q}, in which M is a metal of subgroup VIII, L is a ligand and q, x, y, z are integers, depending on the valence and type of the metal and the number of coordination sites occupied by the ligand L.

According to a preferred embodiment, the hydroformylation catalysts are produced in situ in the reactor used for the hydroformylation reaction.

Another preferred form is the use of a carbonyl generator in which preprepared carbonyl is adsorbed e.g. to activated carbon and only the desorbed carbonyl is passed to the hydroformylation, but not the salt solutions from which the carbonyl is produced. Rhodium compounds or complexes suitable as catalysts are, for example, rhodium(II) and rhodium(III) salts, such as rhodium(III) chloride, rhodium(III) nitrate, rhodium(III) sulfate, potassium-rhodium sulfate, rhodium(II) or rhodium(III) carboxylate, rhodium(II) and rhodium(III) acetate, rhodium(III) oxide, salts of rhodium(III) acid, trisammonium hexachlororhodate(III) etc. Rhodium complexes, such as biscarbonyl rhodium acetylacetonate, acetylacetonatobisethylenerhodium(I) etc. are also suitable.

Ruthenium salts or ruthenium compounds are likewise suitable. Suitable ruthenium salts are, for example, ruthenium(III) chloride, ruthenium(IV), ruthenium(VI) or ruthenium(VIII) oxide, alkali metal salts of ruthenium oxo acids, such as K₂RuO₄ or KRuO₄ or complex compounds, such as, for example, RuHCl(CO)(PPh₃)₃. The metal carbonyls of ruthenium, such as dodecacarbonyl trisruthenium or octadecacarbonyl hexaruthenium, or mixed forms in which CO has been partially replaced by ligands of the formula PR₃, such as Ru(CO)₃(PPh₃)₂, can also be used.

Suitable cobalt compounds are, for example, cobalt(II) chloride, cobalt(II) sulfate, cobalt(II) carbonate, cobalt(II) nitrate, their amine or hydrate complexes, cobalt carboxylates, such as cobalt formate, cobalt acetate, cobalt ethylhexanoate, cobalt naphthanoate, and the cobalt-caprolactamate complex. The carbonyl complexes of cobalt, such as octacarbonyl dicobalt, dodecacarbonyl tetracobalt and hexadecacarbonyl hexacobalt, can also be used here.

The specified and further suitable compounds are known in principle and described adequately in the literature.

Suitable activators which can be used for the hydroformylation are, for example, Brönsted acids, Lewis acids, such as BF₃, AlCl₃, ZnCl₂, and Lewis bases.

The composition of the synthesis gas used comprising carbon monoxide and hydrogen can vary within wide ranges. The molar ratio of carbon monoxide and hydrogen is generally about 5:95 to 95:5, preferably about 40:60 to 60:40. The temperature during the hydroformylation is generally in a range from about 20 to 200° C., preferably about 50 to 190° C. The reaction is generally carried out at a partial pressure of the reaction gas at the selected reaction temperature. In general, the pressure is in a range from about 1 to 700 bar, preferably 1 to 300 bar.

The functionalized polyisobutenes obtained by hydroformylation are advantageously suitable as intermediates for the further processing by functionalization of at least some of the aldehyde functions present therein.

α) Oxocarboxylic Acids

For the further functionalization, the hydroformylated polyisobutenes obtained in step iv) can be reacted with an oxidizing agent to give a polyisobutene functionalized at least partially with carboxy groups.

For the oxidation of aldehydes to carboxylic acids, it is generally possible to use a large number of different oxidizing agents and oxidation methods, which are described, for example, in J. March, Advanced Organic Chemistry, Verlag John Wiley & Sons, 4th edition, p. 701ff. (1992). These include, for example, oxidation with permanganate, chromate, atmospheric oxygen, etc. The oxidation with air/oxygen can take place either catalytically in the presence of metal salts, or in the absence of catalysts. The metals used are preferably those which are capable of changing valency, such as Cu, Fe, Co, Mn, etc. The reaction generally takes place also in the absence of a catalyst. In the case of air oxidation, the conversion can be readily controlled via the reaction time.

To produce the described amphiphilic block copolymers a) of hydrophobic units A and hydrophilic units B, the polyisobutenes obtained are reacted with carboxy function in a further step. Reactions may be with alkylene oxides, esterifications with polyalkylene oxides or amide formations with polyethyleneimines. The reactions take place as described under iii) points β) and δ) to τ).

Which alkylene oxides or polyalkylene oxides are preferably used is described in the section “Hydrophilic units B”.

β) Oxo Alcohols

According to a further suitable embodiment, the hydroformylated polyisobutenes obtained in step iv) can be subjected to a reaction with hydrogen in the presence of a hydrogenation catalyst to give a polyisobutene functionalized at least partially with alcohol groups.

Suitable hydrogenation catalysts are generally transition metals, such as Cr, Mo, W, Fe, Rh, Co, Ni, Pd, Pt, Ru, etc., or mixtures thereof which, to increase the activity and stability, can be applied to supports, such as activated carbon, aluminum oxide, kieselguhr, etc. To increase the catalytic activity, Fe, Co, and preferably Ni, also in the form of the Raney catalysts as metal sponge with a very large surface area can be used.

The hydrogenation of the oxo aldehydes from stage iv) preferably takes place at elevated temperatures and increased pressure, depending on the activity of the catalyst. Preferably, the reaction temperature is about 80 to 150° C. and the pressure is about 50 to 350 bar.

To produce the described block copolymers a), in a further step, the polyisobutene functionalized with alcohol groups is reacted with alkylene oxides by means of graft polymerization. Which alkylene oxides are preferably used is described in the section “Hydrophilic units B”.

χ) Amine Synthesis

According to a further suitable embodiment, the hydroformylated polyisobutenes obtained in step iv) are subjected, for further functionalization, to a reaction with hydrogen and ammonia or a primary or secondary amine in the presence of an amination catalyst to give a polyisobutene functionalized at least partially with amine groups.

Suitable amination catalysts are the hydrogenation catalysts described above in stage β), preferably copper, cobalt or nickel, which can be used in the form of the Raney metals or on a support. Furthermore, platinum catalysts are also suitable.

In the amination with ammonia, aminated polyisobutenes with primary amino functions are obtained. Primary and secondary amines suitable for the amination are compounds of the general formulae R—NH₂ and RR′NH, in which R and R′, independently of one another, are, for example, C₁-C₁₀-alkyl, C₆-C₂₀-aryl, C₇-C₂₀-arylalkyl, C₇-C₂₀-alkylaryl or cycloalkyl.

To produce the described amphiphilic block copolymers a), in a further step, the polyisobutene functionalized with amino groups is reacted with alkylene oxides by means of graft polymerization. Which alkylene oxides are preferably used is described in the section “Hydrophilic Units B”.

Re v) Production of Phosphonic Acid Derivatives

For the functionalization, the reactive polyisobutene can be subjected to a reaction with PX₅ (X═Cl, Br, I) to give a polyisobutene functionalized with a phosphonic acid halide group. For the further functionalization, the derivatized polyisobutene is subjected to a subsequent reaction which is selected from:

α) graft polymerization with at least one alkylene oxide to give a polyisobutene functionalized with phosphonic ester groups,

β) hydrolysis to give a polyisobutene functionalized with phosphonic acid groups, where the phosphonic acid groups are reacted as under a) with alkylene oxides by means of graft polymerization,

χ) reaction with at least one amine to give a polyisobutene functionalized at least partially with phosphonamide groups, which is reacted in a further reaction with alkylene oxides by means of graft polymerization,

δ) reaction with at least one alcohol to give a polyisobutene functionalized with phosphonic ester groups, which is reacted in a further reaction with alkylene oxides by means of graft polymerization,

ε) reaction with at least one polyethyleneimine to give a polyisobutene functionalized at least partially with phosphonamide groups,

φ) reaction with at least one polyalkylene oxide which has at least one hydroxy group to give a polyisobutene functionalized at least partially with phosphonic ester groups,

γ) reaction with at least one polyalkylene oxide which has at least one amino group to give a polyisobutene functionalized at least partially with phosphonamide groups,

η) reaction with at least one polyalkylene oxide which has at least one thio group to give a polyisobutene functionalized at least partially with phosphonic thioester groups,

τ) if, following the reaction of the phosphonic acid halide group, free acid or halide groups are still present, these can also be converted into salts. Suitable cations in salts are primarily alkali metal cations, ammonium ions and alkylammonium ions.

Re χ) and δ)

For further derivatization, the phosphonic acid halide groups can be reacted, for example, with polar reactants such as alcohols or amines. Suitable polar reactants are preferably alcohols ROH or primary amines RNH₂ or secondary amines RR′NH, where R is a linear or branched saturated hydrocarbon radical which carries at least two substituents selected from the group OH, SH, NH₂ or NH₃⁺ and, if appropriate, one or more CH(O) groups and, if appropriate, has nonadjacent —O— and/or —NH-and/or tertiary-N— groups, and R′, independently of one another of R, has the same meaning. Here, both phosphonic acid groups can be reacted, or just one, while the other phosphonic acid group is present as free acid group or as salt. In a further reaction, the free substituents (substituents not reacted with phosphonic acid halide group) are modified by alkoxylation, giving the described block copolymers a).

Re ε)

To produce the described block copolymers a), the phosphonic acid halide groups can be reacted with polyethyleneimines in a polymer-analogous manner where, depending on the reaction procedure, one or more polyisobutene chains per polyethyleneimine chain are joined. The binding takes place via phosphonamide groups. The polyethyleneimines are preformed hydrophilic units B.

Re γ), η) and τ)

To produce the described block copolymers a), the succinic anhydride groups are reacted with polyalkylene oxides in a polymer-analogous manner. Here, the polyalkylene oxides used must have at least one group selected from OH, SH, NH₂ or NH. The polyethylene oxides are preformed hydrophilic units B.

Re γ), η) and τ)

To produce the described block copolymers a), the phosphonic anhydride groups are reacted with polyalkylene oxides in a polymer-analogous manner. Here, the polyalkylene oxides used must have at least one group selected from OH, SH, NH₂ or NH. The polyethylene oxides are preformed hydrophilic units B.

Which alkylene oxides or polyalkylene oxides can preferably be used in each case is described in the section “Hydrophilic units B”.

Re vi) Hydroboration with Subsequent Oxidation

For the functionalization, the reactive polyisobutene can be subjected to a reaction with a (if appropriate, in situ-produced) borane and subsequent oxidation, giving a polyisobutene functionalized with a hydroxy group.

Suitable methods for the hydroboration are described in J. March, Advanced Organic Chemistry, 4th edition, Verlag John Wiley & Sons, pp. 783-789, to which reference is hereby made. Suitable hydroboration reagents are, for example, diborane, which is usually produced in situ by reacting sodium borohydride with BF₃ etherate, diisamylborane (bis[3-methylbut-2-yl]borane), 1,1,2-trimethylpropylborane, 9-borobicyclo[3.3.1]nonane, diisocampheylborane, which are obtainable by hydroboration of the corresponding alkenes with diborane, chloroborane dimethylsulfide, alkyldichloroborane or H₃B—N(C₂H₅)₂.

The hydroboration is usually carried out in a solvent. Suitable solvents for the hydroboration are, for example, acyclic ethers, such as diethyl ether, methyl tert-butyl ether, dimethoxyethane, diethylene glycol dimethyl ether, triethylene glycol dimethyl ether, cyclic ethers, such as tetrahydrofuran or dioxane, and hydrocarbons, such as hexane or toluene or mixtures thereof. The reaction temperature is usually determined by the reactivity of the hydroboration agent and is normally between the melting point and the boiling point of the reaction mixture, preferably in the range from 0° C. to 60° C.

Usually, the hydroboration agent is used in excess based on the alkene. The boron atom preferably adds onto the less substituted and thus sterically less hindered carbon atom.

Usually, the alkylboranes formed are not isolated, but converted directly to the products of value by subsequent reaction. A very important reaction of the alkylboranes is the reaction with alkaline hydrogen peroxide to give an alcohol which preferably corresponds formally to the anti-Markovnikov hydroxylation of the alkene.

To produce the described block copolymers a), in a further step, the polyisobutene functionalized with hydroxy groups is reacted with alkylene oxides by means of graft polymerization. Which alkylene oxides are preferably used is described in the section “Hydrophilic units B”.

Re vii) Reaction with an SO₃ Source

For the functionalization, the reactive polyisobutene can furthermore be reacted with an SO₃ source, forming a polyisobutene with terminal sulfonic acid groups.

The polyisobutenes functionalized with sulfonic acid groups can be produced by reacting the reactive polyisobutenes with an SO₃ source. Suitable SO₃ sources are a mixture of sulfur trioxide and air, sulfur trioxide hydrates, sulfur trioxide amine complexes, sulfur trioxide ether complexes, sulfur trioxide phosphate complexes, oleum, acetyl sulfate, a mixture of sulfur trioxide and acetic anhydride, sulfamic acid, alkyl sulfates or chlorosulfonic acids. The reaction can take place either without a diluent or in any inert anhydrous solvent. Suitable reaction temperatures are in the range from −30° C. to +200° C. and are dependent on the sulfonation reagent used. For example, a sulfonation with acetyl sulfate takes place at low temperatures and elevated temperatures should be avoided since otherwise decomposition of the product can occur. The sulfonation reagent is generally used in a molar ratio to polyisobutene of from 1:1 to 2:1. Preference is given to using acetyl sulfate or a mixture of sulfuric acid and acetic anhydride, where acetyl sulfate is formed in situ, where the polyisobutene functionalized with sulfonic acid groups is formed directly. Some of the other specified sulfonation reagents, e.g. the mixture of sulfur trioxide and oxygen, can firstly form an intermediate sultone, which has to be hydrolyzed to the desired sulfonic acid. One method of producing polyisobutenes functionalized with sulfonic acid groups is disclosed, for example, in WO 01/70830.

As described under v) for the phosphonic acid halide groups (points β-τ), the polyisobutenes functionalized with sulfonic acid groups can also be reacted with alkylene oxides, polyalkylene oxides or polyethyleneimines to give the block copolymers a). Which alkylene oxides or polyalkylene oxides are preferably used is described in the section “Hydrophilic units B”.

Re viii) Functionalization with Amino Groups

For the functionalization, the reactive polyisobutene can be reacted with oxides of nitrogen, in which case, following subsequent hydrogenation, polyisobutenes with terminal amino groups are obtained.

Suitable oxides of nitrogen are, for example, NO, NO₂, N₂O₃, N₂O₄, mixtures of these oxides of nitrogen with one another and mixtures of these oxides with nitrogen with oxygen. Particular preference is given to mixtures of NO or NO₂ with oxygen. Furthermore, the oxides of nitrogen can additionally comprise inert gases, for example nitrogen. The reaction of the polyisobutenes with the oxides of nitrogen generally takes place at a temperature of from −30 to +150° C. in an inert organic solvent. The products obtained are then hydrogenated, preferably by catalytic hydrogenation with hydrogen in the presence of hydrogenation catalysts. The hydrogenation is generally carried out in a temperature range from 20 to 250° C., depending on the reduction system used. The hydrogenation pressure in the catalytic hydrogenation is generally 1 bar to 300 bar. A method of producing polymers terminated with amino groups is disclosed, for example, in WO 97/03946.

To produce the described block copolymers a), in a further step, the polyisobutene functionalized with amino groups is reacted with alkylene oxides by means of graft polymerization. Which alkylene oxides are preferably used is described in the section “Hydrophilic units B”.

Re ix) Addition of Hydrogen Sulfide and Thiols

For the functionalization, the reactive polyisobutene can be subjected to a reaction with hydrogen sulfide or thiols, such as alkyl- or arylthiols, hydroxymercaptans, aminomercaptans, thiocarboxylic acids or silanethiols, giving a polyisobutene functionalized with thio groups.

Suitable hydro-alkylthio additions are described in J. March, Advanced Organic Chemistry, 4th edition, Verlag John Wiley & Sons, pp. 766-767, to which reference is made here in its entirety. The reaction can generally take place either in the absence or in the presence of initiators, and in the absence of electromagnetic radiation. In the case of the addition of hydrogen sulfide, polyisobutenes functionalized with thiol groups are obtained. In the reaction with thiols in the absence of initiators, the Markovnikov addition products onto the double bond are generally obtained. Suitable initiators of the hydro-alkylthio addition are, for example, protic acids and Lewis acids, such as concentrated sulfuric acid or AlCl₃. Furthermore, suitable initiators are those which are capable of forming free radicals. In the case of the hydro-alkylthio addition in the presence of these initiators, the anti-Markovnikov addition products are usually obtained. Furthermore, the reaction can take place in the presence of electromagnetic radiation with a wavelength of from 10 to 400 nm, preferably 200 to 300 nm.

To produce the described block copolymers a), in a further step, the polyisobutene functionalized with thiol groups is reacted with alkylene oxides by means of graft polymerization. Which alkylene oxides are preferably used is described in the section “Hydrophilic units B”.

Hydrophilic Units B

The amphiphilic polymers a) consist of one or more hydrophobic units A and one or more hydrophilic units B. The hydrophobic units A consist of reactive polyisobutenes modified with terminal, polar groups. These functionalizations of the reactive polyisobutenes are described above. To introduce the hydrophilic units B, the functionalized polyisobutenes (units A) are reacted, depending on the nature of their polar group(s), either with alkylene oxides by means of graft polymerization or in polymer-analogous reactions with polyalkylene oxides or polyethyleneimines. The way in which the hydrophilic units are introduced has been described above. Irrespective of the type of introduction, the same compositions apply for the hydrophilic units B of polyethylene oxides.

Amphiphilic block copolymers a) can be obtained by reacting the functionalized polyisobutene with alkylene oxide or by polymer-analogous reaction with polyalkylene oxide. Which method is chosen depends on the type of functionalization of the reactive polyisobutene.

Alkylene oxides used for the reaction with functionalized polyisobutene are preferably ethylene oxide or ethylene oxide/propylene oxide, preferably with a fraction of from 0 to 50% by weight propylene oxide, particularly preferably with a fraction of from 0 to 20% by weight propylene oxide, very particularly preferably of ethylene oxide. Here, the alkylene oxide block which forms may be a random copolymer, a gradient copolymer, an alternating or a block copolymer of ethylene oxide and propylene oxide. Besides ethylene oxide and propylene oxide, the following pure alkylene oxides or else mixtures may be used: 1,2-butene oxide, 2,3-butene oxide, 2-methyl-1,2-propene oxide (isobutene oxide), 1-pentene oxide, 2,3-pentene oxide, 2-methyl-1,2-butene oxide, 3-methyl-1,2-butene oxide, 2,3-hexene oxide, 3,4-hexene oxide, 2-methyl-1,2-pentene oxide, 2-ethyl-1,2-butene oxide, 3-methyl-1,2-pentene oxide, decene oxide, 4-methyl-1,2-pentene oxide, styrene oxide or be formed from a mixture of oxides of industrially available raffinate streams.

Either polyalkylene oxides or polyethyleneimines can be used as hydrophilic unit B. Preference is given to polyalkylene oxides, based on ethylene oxide, propylene oxide, butylene oxide or else further alkylene oxides. Further alkylene oxides which may be used are the following pure alkylene oxide or else mixtures: 1-butene oxide, 2,3-butene oxide, 2-methyl-1,2-propene oxide (isobutene oxide), 1-pentene oxide, 2,3-pentene oxide, 2-methyl-1,2-butene oxide, 3-methyl-1,2-butene oxide, 2,3-hexene oxide, 3,4-hexene oxide, 2-methyl-1,2-pentene oxide, 2-ethyl-1,2-butene oxide, 3-methyl-1,2-pentene oxide, decene oxide, 4-methyl-1,2-pentene oxide, styrene oxide or mixture of oxides which are formed from industrially available raffinate streams. In addition, polyglycerol and poly-THF can also be used.

Depending on the type of monomer building blocks, the polyalkylene oxides comprise the following structural units:

—(CH₂)₂-0-, —(CH₂)₃-0-, —(CH₂)₄-0-, —CH₂CH(R⁹)-0-, —CH₂—CHOR¹⁰—CH₂-0-

where R⁹ is C₁-C₂₄-alkyl;

R¹⁰ is hydrogen, C₁-C₂₄-alkyl, R⁹—C(═O)—, R⁹—NH—C(═O)—.

Here, the structural units may either be homopolymers or random copolymers, gradient copolymers, alternating or block copolymers.

Preferably, the hydrophilic units B used are compounds of the following formula (II)

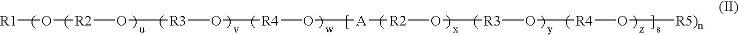

where the variables, independently of one another, have the following meanings:

R¹: hydrogen, C₁-C₂₄-alkyl, R⁶—C(═O)—, R⁶—NH—C(═O)—, polyalcohol radical;

R⁵: hydrogen, C₁-C₂₄-alkyl, R⁶≠C(∇O)—, R⁶—NH—C(═O)—;

R² to R⁴: —(CH₂)₂—, —(CH₂)₃—, —(CH₂)₄—, —CH₂—CH(R⁶)—, —CH₂—CHOR⁷—CH₂—;

R⁶: C₁-C₂₄-alkyl;

R⁷: hydrogen, C₁-C₂₄-alkyl, R⁶—C(═O)—, R⁶—NH—C(═O)—;

A: —C(═O)—O, —C(═O)-D-C(═O)—O, —CH₂—CH(—OH)-D-CH(—OH)—CH₂—O,—C(═O)—NH-D-NH—C(═O)—O;

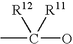

D: —(CH₂)ₜ—, arylene, opt. substituted;

R¹¹, R¹²: hydrogen, C₁-C₂₄-alkyl, C₁-C₂₄-hydroxyalkyl, benzyl or phenyl;

n: is 1 when R¹ is not a polyalcohol radical or is 1 to 500 when R′ is a polyalcohol radical

s=0 to 1000; t=1 to 12; u=1 to 2000; v=0 to 2000; w=0 to 2000;

x=0 to 2000; y=0 to 2000; z=0 to 2000.

Alkyl radicals for R⁶ and R¹¹ and R¹² which may be mentioned are branched or unbranched C₁-C₂₄-alkyl chains, preferably methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, 2-ethylhexyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl or n-eicosyl.

Preferred representatives of the abovementioned alkyl radicals which may be mentioned are branched or unbranched C₁-C₁₂—, particularly preferably C₁-C₆-alkyl chains.

Preference is given to polyalkylene oxides which are composed of repeating alkylene oxide units, such as of ethylene oxide or ethylene oxide/propylene oxide units, preferably with a fraction of from 0 to 50% propylene oxide, particularly preferably with a fraction of from 0 to 20% propylene oxide units. In this connection, it may be a random copolymer, a gradient copolymer, an alternating copolymer or a block copolymer of ethylene oxide and propylene oxide. A very particularly preferred polyalkylene oxide is polyethylene oxide.

The number-average molecular weight of the polyalkylene oxides is in the range from 150 to 50 000, preferably in the range from 200 to 50 000, particularly preferably in the range from 500 to 30 000, very particularly preferably in the range from 800 to 15 000.

In a further embodiment, the polyalkylene oxides may be monoalkyl polyethylene oxide (alkyl is, for example, methyl, ethyl, C₁₂, C₁₈, etc.), monoester polyethylene oxide (ester is, for example, R—(C(═O)—, where R═C₄-C₂₄), monoaminopolyethylene oxide, monothiopolyethylene oxide, diaminopolyethylene oxide (cf. JP-A-09272796, PEO-diamine), etc.

Suitable polyethylene oxides (preformed hydrophilic units B) are, for example, the commercially available Pluriol® E grades (BASF), suitable polypropylene oxides are, for example, the commercially available Pluriol® P grades (BASF), suitable mixed copolymers of ethylene oxide and propylene oxide are, for example, the commercially available Pluriol® PE or Pluriol® RPE grades (BASF), suitable monoalkylpolyethylene oxides are, for example, the commercially available Lutensol® grades (BASF).

Besides straight-chain homopolymers or copolymers, it is also possible to use branched homopolymers or copolymers as hydrophilic unit B. Branched polymers can be produced by, for example, adding ethylene oxide and, if appropriate, also propylene oxide and/or butylene oxide onto polyalcohol radicals, e.g. onto pentaerythritol, glycerol, trimethylolpropane or onto sugar alcohols such as sucrose, D-sorbitol and D-mannitol, but also onto polysaccharides such as cellulose and starch. The alkylene oxide blocks can be in random distribution, in gradient distribution, alternating or sequential.

However, it is also possible to use polyesters of polyalkylene oxides and aliphatic or aromatic dicarboxylic acids, for example oxalic acid, succinic acid, adipic acid and terephthalic acid, with molar masses of from 1500 to 25 000, as described, for example, in EP-A-0 743 962, as polyether-containing compound. Furthermore, it is also possible to use polycarbonates by reacting polyalkylene oxides with phosgene or carbonates such as, for example, diphenyl carbonate, and polyurethanes by reacting polyalkylene oxides with aliphatic and aromatic diisocyanates.

Furthermore, polyalkylene oxides which can be used are also homopolymers and copolymers of polyalkylene-oxide-containing ethylenically unsaturated monomers, such as, for example, polyalkylene oxide(meth)acrylates, polyalkylene oxide vinyl ethers, polyalkylene oxide(meth)acrylamides, polyalkylene oxide allyamines or polyalkylene oxide vinylamines. Copolymers of such monomers with other ethylenically unsaturated monomers can of course also be used. Suitable polyalkylene oxide allyl ethers are, for example, the Pluriol® AR grades (BASF).

As hydrophilic unit B, however, it is also possible to use reaction products of polyethyleneimines with alkylene oxides. The alkylene oxides used in this case are preferably ethylene oxide, propylene oxide, butylene oxide and mixtures thereof, particularly preferably ethylene oxide. Polyethyleneimines which can be used are polymers with number-average molecular weights of from 300 to 20 000, preferably 500 to 10 000, very particularly preferably 500 to 5000. The weight ratio between alkylene oxide used and polyethyleneimine is in the range from 100:1 to 0.1:1, preferably in the range 50:1 to 0.5:1, very particularly preferably in the range 20:1 to 0.5:1.

To produce the hydrophilic polyalkylene oxide units B, use is made of alkoxylation catalysts. This applies irrespective of the type of bonding to the hydrophobic functionalized polyisobutene unit A either as preformed polyalkylene oxide unit introduced in a polymer-analogous manner, or polyalkylene oxide unit forming during the alkoxylation by grafting. Alkoxylation catalysts which can be used are bases, for example alkali metal hydroxides or alkali metal alkoxides, but also Lewis acids, for example BF₃, SbCl₅, SnCl₄×2H₂O, BF₃×H₃BO₄, or BF₃ dietherate. Particularly suitable alkoxylation catalysts are double hydroxide clays, such as hydrotalcite, which can in particular be modified with additives, as described in DE-A 43 25 237.

Depending on the choice of alkoxylation catalysts, specific properties of the alkoxylates result in each case, especially with regard to the distribution of the degree of alkoxylation. Thus, when using the last-mentioned double hydroxide clays, alkoxylation products with a narrow molecular weight distribution or homolog distribution are obtained, which are particularly suitable for use in the block copolymers according to the invention.

The advantageous properties described above, in particular with regard to the degree of alkoxylation, are also achieved through use of double metal cyanide (DMC) compounds, as are described, for example, in DE-A 102 43 361 as alkoxylation catalysts.

The amphiphilic block copolymers a) used for the preparations according to the invention consist of at least one hydrophilic unit A, formed from reactive polyisobutenes, and at least one hydrophilic unit B, formed from a polyalkylene oxide or a polyethyleneimine. For the linkage of one or more units A with one or more units B, the hydrophobic units A comprise at least one polar functional group as anchor group. Depending on the type of anchor group(s), the functionalized polyisobutenes are reacted either with alkylene oxides in a graft polymerization or in a polymer-analogous reaction with polyalkylene oxides or polyethyleneimines.

The linkage of the hydrophobic unit A and of the hydrophilic unit B preferably takes place in a polymer-analogous reaction. Here, one or more functionalized polyisobutenes are reacted with polyalkylene oxides or polyethyleneimines. In polymer-analogous reactions, therefore, preformed blocks A and B are used.

Particular preference is given to using polyalkylene oxides as preformed blocks B. Preference is given to polyalkylene oxides which are composed of repeating alkylene oxide units, such as ethylene oxide or ethylene oxide/propylene oxide units, preferably with a fraction of from 0 to 50% propylene oxide units, particularly preferably with a fraction of from 0 to 20% propylene oxide units. This may be a random copolymer, a gradient copolymer, an alternating or a block copolymer of ethylene oxide and propylene oxide. A very particularly preferred polyalkylene oxide is polyethylene oxide.

The molecular weight of the polyalkylene oxides is in the range from 150 to 50 000 (number-average), preferably in the range from 200 to 50 000, particularly preferably in the range from 500 to 30 000, very particularly preferably in the range from 800 to 15 000.

Besides polyalkylene oxides, such as polyethylene oxide, polypropylene oxide, mixed copolymers of EO and PO. The mixed copolymers of EO and PO may be a random copolymer, a gradient copolymer, an alternating or a block copolymer of ethylene oxide and propylene oxide. In a further embodiment, the polyalkylene oxides may be monoalkyl polyethylene oxide (alkyl=methyl, ethyl, C₁₂, C₁₈, etc.), monoester polyethylene oxide (ester=R—(C(═O)—, where R═C₄-C₂₄), monoaminopolyethylene oxide, monothiopolyethylene oxide, diaminopolyethylene oxide (cf. JP-A-09272796, PEO-diamine, etc.

Suitable polyethylene oxides (preformed hydrophilic units B) are, for example, the Pluriol® E grades from BASF AG, suitable polypropylene oxides are, for example, the Pluriol® P grades from BASF AG, suitable mixed copolymers of ethylene oxide and propylene oxide are, for example, the Pluriol® PE or Pluriol® RPE grades from BASF AG, suitable monoalkyl polyethylene oxides are, for example, the Lutensol® grades from BASF AG.

For the preparations according to the invention, preference is given to using hydrophobic units A formed from reactive polyisobutenes which have at least one polar functional group which is capable of polymer-analogous reactions with hydrophilic blocks B. Preferred hydrophobic units A are selected from

phosphated polyisobutenephenols described under i), phosphated hydrogenated polyisobutenephenols, phosphated polyisobutenephenols which have been subjected beforehand to a Mannich reaction, sulfated polyisobutenephenols, sulfated hydrogenated polyisobutenephenols, sulfated polyisobutenephenols which have been subjected beforehand to a Mannich reaction,

functionalized polyisobutene described under iii) which are produced by means of an ene reaction. Suitable enophiles are fumaryl dichloride, fumaric acid, maleoyl dichloride, maleic anhydride and maleic acid, preferably maleic anhydride and maleic acid, very particularly maleic anhydride,

polyisobutenes functionalized with carboxy groups described under iv),

polyisobutenes functionalized with phosphonic acid groups described under v),

polyisobutenes functionalized with sulfonic acid groups described under vii).

Particularly preferred hydrophobic units A are selected from polyisobutenes functionalized with phosphonic acid, sulfonic acid and maleic anhydride groups. Hydrophobic units A that are very particularly suitable for the preparations according to the invention are polyisobutenes functionalized with succinic anhydride groups (PIBSA).

The polyisobutene block here has an average molar mass of Mₙ of 150 to 50 000, preferably of Mₙ=200 to 20 000, particularly preferably from Mₙ=450 to 5000.

In a particular embodiment of the amphiphilic block copolymers a) used according to the invention, their hydrophobic units A consist of polyisobutenesuccinic anhydrides (PIBSA) and their hydrophilic units B consist of polyalkylene oxides.

Particularly preferred polyalkylene oxides are polyethylene oxide, polypropylene oxide, mixed copolymers of EO and PO, monoalkylpolyethylene oxides and monoalkylpolypropylene oxides. Very particular preference is given to amphiphilic block copolymers A composed of polyethylene oxides or monoalkylpolyethylene oxides and PIBSA. Said reaction products form linear AB and ABA structures if the polyisobutenesuccinic anhydride used is a polyisobutene functionalized only at one chain end with a succinic anhydride group. If, for example, a polyisobutene functionalized at both chain ends (formed from the living cationic polymerization) is used, then linear BAB and (AB)ₙ structures can also be formed. Where n is an integer where n=2-100, preferably n=2-50 and particularly preferably n=2-10.

The hydrophilic units B of the block copolymers preferably have a number-average molecular weight Mₙ in the range from 150-50 000, preferably from 500-30 000 and in particular from 800-15 000 g/mol.

Preferably, the amphiphilic polymer a) has structures of the empirical formula AₚB_{q}, in which p and q, independently of one another, are 1 to 8.

In a preferred embodiment of the invention, the amphiphilic polymer a) has a triblock structure ABA.

In a further embodiment, hydrophilic units B which can be used are branched or comb-like polyalkylene oxides. Branched or comb-like polyalkylene oxides are formed by alkoxylation of polyalcohols. Polyalcohols are, for example, glycerol, trimethylolpropane, pentaerythritol, glucose, sucrose, generally carbohydrates, starch and starch hydrolyzates or polyvinyl alcohols.

Possible hydrophilic units are, for example, the reaction products of polyhydric alcohols, for example glycerol, with alkylene oxide, for example ethylene oxide. This produces comb-like molecules, where the glycerol structures form the “handle” and the polyethylene oxide chains form the “teeth” of the comb. The linkage to the hydrophobic units A can then take place via the free OH groups of the polyalkylene oxide chain ends.

Particularly preferred structures are diblock copolymers AB and triblock copolymers ABA composed of PIBSA as hydrophobic block A and of polyethylene oxide and monoalkylpolyethylene oxide as hydrophilic block B.

The synthesis of triblock copolymers of the structure ABA preferably starts from a succinic anhydride which comprises a covalently bonded polyisobutylene block, i.e. from polyisobutenesuccinic anhydride (PIBSA). This is the block A which is bonded to succinic anhydride via a covalent C—C bond. Succinic anhydride takes on the function of a linker which joins blocks A and B together. PIBSA is reacted in a polymer-analogous reaction with polyethylene oxides to give the half-esters. The reaction of PIBSA with polyalkylene glycols thus consists in an esterification.

The diagrammatic reaction of PIBSA and a polyethylene oxide serves as an example:

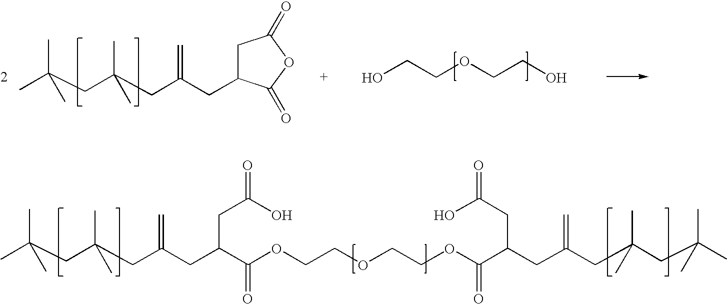

Depending on the use, a certain ratio between hydrophobic PIB block and hydrophilic polyalkylene oxide block is chosen. Another way of controlling the desired effect is to use diblock or triblock copolymers or other block structures. In individual cases, a mixture of the copolymers described here is advantageous. Mixture variants may be of variable hydrophobic block, variable hydrophilic block, variable structure (AB or ABA or AₚB_{q} where p and q, independently of one another, are from 3 to 8 or comb structures).

In a preferred embodiment, besides the amphiphilic block copolymers a) which can also comprise remains of starting materials, the cosmetic preparations also comprise further polyalkylene oxides, in particular polyethylene oxides, monoalkylpolyethylene oxides or branched polyalkylene oxides and/or free, preferably nonfunctionalized PIB. Free PIB is understood as meaning PIB which has not been covalently linked to alkylene oxide, polyalkylene oxide or polyethyleneimine. Preferably, this free PIB is not functionalized with a polar group.

If the amphiphilic block copolymer is present with free PIB in the preparations, then the weight ratio of amphilic block copolymer a) to free PIB is preferably from 100:1 to 0.1:1, particularly preferably 50:1 to 0.2:1, very particularly preferably 20:1 to 0.2:1.

If the amphiphilic block copolymer is present together with free polyethylene oxide, monoalkylpolyethylene oxide and/or branched polyalkylene oxide in the preparations, then the weight ratio of amphiphilic block copolymer a) to free polyethylene oxide, monoalkylpolyethylene oxide and/or branched polyalkylene oxide is in the range from 100:1 to 0.1:1, preferably in the range 50:1 to 0.2:1, very particularly preferably in the range 20:1 to 0.2:1.

Examples of the block copolymers present in the preparations according to the invention are block copolymers composed of at least one hydrophobic block A consisting of polyisobutene and at least one hydrophilic block B consisting of polyalkylene oxide. The structure of the block copolymers can here generally be described by AₚB_{q} (where p and q, independently of one another are from 1 to 8).

It is also possible to use block copolymers with a comb structure, where A is a polyisobutene block with an average molar mass Mₙ of from 150 to 50 000, and

B is a polyalkylene oxide block with an average molar mass Mₙ of from 150 or 200 to 50 1000.

In a particular embodiment, the block copolymers a) for the preparations according to the invention can be provided beforehand in water. Particular preference is given to aqueous preparations which comprise block copolymers composed of polyisobutene functionalized with succinic anhydride groups (PIBSA) as hydrophobic block A and of polyethylene oxide or monoalkylpolyethylene oxide as hydrophilic block B of structure ABA or AB, where

A is a polyisobutene block with an average molar mass Mₙ of from 450 to 5000, and

B is a polyalkylene oxide block with an average molar mass Mₙ of from 800 to 15 000.

A preferred embodiment of the invention are cosmetic preparations according to the invention where the hydrophobic unit A and the hydrophilic unit B have a number-average molar mass Mₙ of from 150 to 50 000 g/mol.

Another preferred embodiment of the invention are cosmetic preparations according to the invention where Mₙ of the hydrophobic unit A is in the range from 200 to 20 000 g/mol and Mₙ of the hydrophilic unit B is in the range from 500 to 30 000 g/mol. Another preferred embodiment are cosmetic preparations according to the invention where Mₙ of the hydrophobic unit A is in the range from 450 to 5000 g/mol and Mₙ of the hydrophilic unit B is in the range from 800 to 15 000 g/mol.

Preference is given to hydrophobic units with Mₙ of at least 150 g/mol, particularly preferably of at least 200 g/mol and in particular of at least 450 g/mol and of at most 50 000 g/mol, particularly preferably of at most 20 000 g/mol and in particular of at most 5000 g/mol.

Preference is given to hydrophilic units with Mₙ of at least 150 g/mol, particularly preferably of at least 200 g/mol, in particular of at least 500 g/mol and most preferably of at least 800 g/mol and of at most 50 000 g/mol, particularly preferably of at most 30 000 g/mol and in particular of at most 15 000 g/mol.

In the preparations according to the invention it is possible to use amphiphilic block copolymers a) which are obtained through the linking of hydrophilic units of an arbitrary aforementioned molecular weight Mₙ with hydrophobic units of an arbitrary aforementioned molecular weight.

In the preparations according to the invention, any mixtures of different amphiphilic block copolymers a) with varying respective stoichiometry AₚB_{q} and/or structure (block, comb etc.) and/or of varying respective molecular weights of the hydrophobic and hydrophilic units A and B can be used.

In the preparations according to the invention, unreacted polyalkylene oxides, polyisobutene, reactive polyisobutene and functionalized polyisobutene may also be present. Polyalkylene oxides, monoalkylpolyethylene oxides, branched polyalkylene oxides, polyisobutene, reactive polyisobutene and functionalized polyisobutene can also be added to the preparations in a targeted manner.

It may also be advantageous to add partially or completely hydrogenated polyisobutene to the preparations. Such hydrogenated polyisobutenes are described, for example, in the unpublished German patent application with the application number DE 102005022021.5 and the unpublished international application with the application number PCT/EP2006/004461, to which reference is made here in their entirety.

It may be advantageous to provide mixtures of amphiphilic block copolymers a) and further substances selected from polyalkylene oxides, monoalkylpolyethylene oxides, branched polyalkylene oxides, polyisobutenes, reactive polyisobutenes, hydrogenated polyisobutene and functionalized polyisobutene in aqueous phase in order then to use them in the preparations according to the invention.

The amphiphilic block copolymer can be used in the preparations without a diluent, in solution or in dispersion. Suitable solvents and dispersants are all cosmetically acceptable solvents, in particular water and mixtures of water and alcohols.

Emulsions based on the amphiphilic block copolymers a) produce a very pleasant feel to the touch on the surfaces treated therewith, such as, for example, the skin, and, compared to the prior art, have a very high salt stability, i.e. stability even in the case of high electrolyte concentrations.

Emulsions according to the invention can have particles with diameters of less than one μm and form multiphase emulsions (MPE), which leads to advantageous, increased transparency compared with the preparations of the prior art. In the field of cosmetic preparations, products with increased transparency are often preferred. A further special feature of the emulsions according to the invention is that they can be provided with a multimodal, preferably bimodal, particle size distribution.

It is further advantageous that the amphiphilic block copolymers a) in cosmetic preparations can also assume the role of a thickener, in particular in preparations with increased salt concentration and/or pigment concentration. Thus, if appropriate, the number of required ingredients of a preparation can be reduced or the addition of rheology modifiers can be rendered superfluous.

A further advantage of the preparations according to the invention is the enhancement of the effect of other ingredients of the preparations, in particular of the active ingredients present. This is then termed a so-called boosting effect. The preparations have such boosting effects, for example, in the presence of UV photoprotective filters, such as, for example, TiO2, i.e. the sun protection factor (SPF) is increased compared with the presence of TiO₂ in the absence of the amphiphilic block copolymers a). This boosting effect also arises in the case of the common presence of amphiphilic block copolymer a) and other cosmetic and dermatological active ingredients.

A further advantage of the preparations according to the invention is that the active ingredients, such as, for example, vitamins or pigments in the case of the simultaneous presence of the amphiphilic block copolymers a) are present in a very uniform and finely divided form.

Cosmetic Preparations

The cosmetic preparations according to the invention comprise the amphiphilic block copolymer a) in an amount in the range from 0.01 to 15% by weight, preferably at least 0.1 and at most 10, further preferably at most 5 and most preferably a concentration of 0.2 to at most 3.5% by weight, based on the weight of the cosmetic preparation.

The cosmetic preparations according to the invention can be in the form of O/W emulsions, hydrodispersion formulations, solids-stabilized formulations, stick formulations, PIT formulations, creams, foams, sprays (pump spray or aerosol), gels, gel sprays, lotions, oils, oil gels or mousses and be formulated accordingly with customary further auxiliaries.

Preferred cosmetic preparations for the purposes of the present invention are gel creams, hydroformulations, stick formulations, cosmetic oil and oil gels, mascara, self-tanning compositions, face care compositions, body care compositions, after sun preparations, hair-shaping compositions, hair-setting compositions, hair gels and compositions for decorative cosmetics.

The invention provides finely divided emulsions comprising the components a) to d) according to claim 1. Such finely divided emulsions may be PIT emulsions and characterized by high storage stability, i.e. even at elevated temperature, neither agglomeration of the droplets nor separation of the preparation takes place.

Skin Cosmetic Preparations

Cosmetic preparations according to the invention which may be mentioned are, for example, skin cosmetic preparations, in particular those for the care of the skin. These are present in particular as O/W skin creams, day and night creams, eye creams, face creams, antiwrinkle creams, mimic creams, moisturizing creams, bleaching creams, vitamin creams, skin lotions, care lotions and moisturizing lotions.

Furthermore, they are suitable for skin cosmetic preparations, such as face tonic, face masks, deodorants and other cosmetic lotions and for use in decorative cosmetics, for example as concealing stick, stage make-up, in mascara and eye shadows, lipsticks, kohl pencils, eyeliners, make-up, foundations, blushes and powders and eyebrow pencils, washing, showering and bath preparations.

Furthermore, the preparations according to the invention can be used in nose strips for pore cleansing, in antiacne compositions, repellants, shaving compositions, hair-removal compositions, personal hygiene compositions, footcare compositions, and in babycare.

Besides the components a) to d), the skin cosmetic preparations according to the invention can also comprise further active ingredients and auxiliaries customary in skin cosmetics, as described below. These include preferably emulsifiers different from b), preservatives, perfume oils, cosmetic active ingredients, such as phytantriol, vitamin A, E and C, retinol, bisabolol, panthenol, natural and synthetic photoprotective agents, bleaches, colorants, tinting agents, tanning agents, collagen, protein hydrolyzates, stabilizers, pH regulators, dyes, salts, thickeners, gel formers, consistency regulators, silicones, humectants, conditioners, refatting agents and further customary additives.

Cosmetically acceptable polymers can also be added to the preparations according to the invention if specific properties are to be set. To improve certain properties, such as, for example, the feel to the touch, the spreading behavior, the water resistance and/or the binding of active ingredients and auxiliaries such as pigments, the preparations can additionally also comprise conditioning substances based on silicone compounds. Suitable silicone compounds are, for example, polyalkylsiloxanes, polyarylsiloxanes, polyarylalkylsiloxanes, polyether siloxanes or silicone resins.

In one embodiment of the invention, the preparations according to the invention comprise no further conditioning polymers since the combined presence of components a) to d) already leads to a good conditioning effects. Further possible ingredients of the preparations according to the invention are described below.

Hair Cosmetic Preparations

Hair cosmetic preparations according to the invention are neutralizers for permanent waves, curl relaxers, styling wrap lotions, hair-setting compositions, hair gels, hair tonics, hair foams, hair mousses, shampoos, hair-shaping compositions and hair colorants. A preferred embodiment is preparations which are in the form of sprays or hair foams.

Besides the components a) to d), a hydrous standard hair spray formulation for setting the hair has, for example, also 2 to 10% by weight of a setting polymer, ethanol, water and, as propellant gas(es), dimethyl ether and/or propane/n-butane and/or propane/isobutane.

Component with an HLB value in the range from 8 to 20 suitable as emulsifier

Components b) suitable for use in the preparations according to the invention are emulsifiers with an HLB value of from 8 to 20, preferably from 8 to 17 and particularly preferably from 10 to 17.

Component b) is present in the preparations according to the invention, based on the overall preparation, in an amount of from 0.01 to 10% by weight, preferably 0.1 to 5% by weight and in particular 0.5 to 2.5% by weight.

With the help of the HLB value (in accordance with W. C. Griffin, J. Soc. Cosmetic Chem.1 (1949) 311), emulsifiers can be classified according to the ratio of hydrophilic to lipophilic groups (HLB=hydrophilic-lipophilic balance).

Compounds suitable as component c) are described, for example, in Karl-Heinz Schrader, Grundlagen und Rezepturen der Kosmetika [Fundamentals and Formulations of Cosmetics], 2nd edition, Verlag Hüthig, Heidelberg, pp. 395-399, to which reference is made here in its entirety. Determination of the HLB value of emulsifiers is known to the person skilled in the art and described, for example, on p. 394 of the abovementioned literature reference.

Examples of components c) are given in the table below:

| | | | nonionogenic | |
|---|---|---|---|---|
| | | | anion-active | |
| Tradename | Manufacturer | Chemical | cation-active | HLB |
| G-2125 | 1 | tetraethylene glycol | n | 9.4 |
| | | monolaurate | | |
| Brij 30 | 1 | polyoxyethylene lauryl ether | n | 9.5 |
| Tween 61 | 1 | polyoxyethylene sorbitan | n | 9.6 |
| | | monostearate | | |
| Gelatine | | “Pharmagel B” | n | 9.8 |
| Tween 81 | 1 | polyoxyethylene sorbitan | n | 10.0 |
| | | monooleate | | |
| Arlypon OAG | 2 | fatty alcohol polyglycol ether | n | 10.0 |
| G-3806 | 1 | polyoxyethylene cetyl ether | n | 10.3 |
| Tween 65 | 1 | polyoxyethylene sorbitan | n | 10.5 |
| | | tristearate | | |
| Methylcellulose | — | methocel 15 cps | | 10.5 |
| Lamecreme SA 7 | 2 | fatty alcohol glycol ether | n | 10.9 |
| Tween 85 | 1 | polyoxyethylene sorbitan | n | 11.0 |
| | | trioleate | | |
| G-1790 | 1 | polyoxyethylene lanolin derivative | n | 11.0 |
| Myrj 45 | 1 | polyethylene glycol | n | 11.2 |
| | | monostearate | | |
| Arlypon OA8 | 2 | polyoxyethylene oleyl | | 11.3 |
| | | alcohol ether | | |
| | 4 | polyethylene glycol- | n | 11.4 |
| | | 400 monooleate | | |
| Cremophor S9 | 7 | polyethylene glycol- | n | 11.6 |
| | | 400 monostearate | | |
| G-2161 | 1 | polyethylene glycol- | n | 11.6 |
| | | 400 monostearate | | |
| Atlox 3300 | 1 | alkylaryl sulfonate | n | 11.7 |
| Lamecreme LPM | 2 | glycerol monodistearate | a | 12.0 |
| Atolx 3300 | | triethanolamine oleate | a | 12.0 |
| G-3910 | 1 | polyoxyethylene oleyl ether | n | 12.2 |
| G-2127 | 1 | polyoxyethylene monolaurate | n | 12.8 |
| Renex 690 | 1 | polyoxyethylene alkyl aryl ether | n | 13.0 |
| Lamecreme AOM | 2 | glycerol monodistearate | n | 13.0 |
| Lamecreme CSM | 2 | glycerol monodistearate | a | 13.0 |
| Lamecreme ZEM | 2 | glycerol monodistearate | a | 13.0 |
| Renex 690 | 4, 6 | polyethylene glycol- | n | 13.1 |
| | | 400 monooleate | | |
| Tragacanth USP | — | | n | 13.2 |
| Cremophor EL | 7 | polyoxyethylene-castor oil | n | 13.3 |
| G-1284 | 1 | polyoxyethylene-castor oil | n | 13.3 |
| G-1425 | 1 | polyethylene glycol sorbitol- | n | 8.0 |
| | | lanolin derivative | | |
| Acacia | — | USP | n | 8.0 |
| G-3608 | 1 | polyoxypropylene stearate | n | 8.0 |
| Span 20 | 1 | sorbitan monolaurate | n | 8.6 |
| Arlacel 20 | 1 | sorbitan monolaurate | n | 8.6 |
| G-2111 | 1 | polyoxyethylene oxypropylene | n | 9.0 |
| | | oleate | | |
| Tween 21 | 1 | polyoxyethylene sorbitan | n | 13.3 |
| | | monolaurate | | |
| Renex 20 | 1 | polyoxyethylene ester of | n | 13.5 |
| | | mixed fatty acids and resin | | |
| | | acids | | |
| Lamecreme KSM | 2 | glycerol monodistearate | a | 14.0 |
| G-1441 | 1 | polyoxyethylene sorbitol- | n | 14.0 |
| | | lanolin derivative | | |
| Lamacit 877 | 2 | polyoxyethylene | | 14.7 |
| | | alkylphenol ether | | |
| G-7596 J | 1 | polyoxyethylene | n | 14.9 |
| | | sorbitan monolaurate | | |
| Lamacit CA | 2 | polyoxyethylene fatty | | 14.9 |
| | | alcohol ether | | |
| Lamacit GML-12 | 2 | polyoxyethylene glycerol | n | 15.0 |
| | | monolaurate | | |
| Tween 60 | 1 | polyoxyethylene sorbitan | n | 15.0 |
| | | monolaurate | | |
| Myrj 49 | 1 | polyoxyethylene monostearate | n | 15.0 |
| Cremophor O | 7 | polyoxyethylene fatty | n | 16.0 |
| | | alcohol ether | | |
| G-1471 | 1 | polyoxyethylene sorbitol- | n | 16.0 |
| | | lanolin derivative | | |
| | | | n | 16.0 |
| Cetomacrogol-1000 | 8 | polyethylene glycol-100 | n | 16.1 |
| | | monocetyl ether | | |
| Lamacit GMO-25 | 2 | poloxyethylene glycerol | n | 16.2 |
| | | monooleate | | |
| Lamacit GML-20 | 2 | polyoxyethylene glycerol | n | 15.0 |
| | | monolaurate | | |
| Tween 20 | 1 | polyoxyethylene sorbitan | n | 16.7 |
| | | monolaurate | | |
| Brij 35 | 1 | polyoxyethylene lauryl ether | n | 16.9 |
| Emulsifier GL-120 | 2 | pentarythritol lanolin | n | 17.0 |
| | | polyglycol ether | | |
| — | | sodium oleate | a | 18.0 |
| — | | potassium oleate | a | 20.0 |

Manufacturer:
(1) ICI, Essen
(2) Henkel, Dusseldorf
(3) Th, Goldschmidt, Essen
(4) Kessler Chem. Comp. Inc., Philadelphia
(5) Emulsol Corp., Chicago
(6) Glyco-Products Inc., New York
(7) BASF, Ludwigshafen
(8) Cyclo Chem. Ltd., London

Component c) is present in the preparations according to the invention in an amount of at least % by weight, preferably at least, particularly preferably at least and at most, preferably at most and particularly preferably at most.

Oils, fats and waxes

The preparations according to the invention comprise an oil phase and/or fat phase c). For the purposes of this invention, this term is understood as meaning all cosmetically acceptable oils, fats and waxes.

A particular advantage of the present invention is that when using amphiphilic polymer a) and emulsifier b), the required amount of further oils, fats or waxes c) can be significantly less than in customary preparations, where the application properties are at least equally as good or even better.

Constituents of the oil phase and/or fat phase of the preparation according to the invention are advantageously selected from the group of lecithins and of fatty acid triglycerides, namely the triglycerol esters of saturated and/or unsaturated, branched and/or unbranched alkanecarboxylic acids of chain length from 8 to 24, in particular 12 to 18, carbon atoms. The fatty acid triglycerides can, for example, advantageously be selected from the group of synthetic, semisynthetic and natural oils, such as, for example, olive oil, sunflower oil, soybean oil, peanut oil, rapeseed oil, almond oil, palm oil, coconut oil, castor oil, wheatgerm oil, grapeseed oil, thistle oil, evening primrose oil, macadamia nut oil and the like. Further polar oil components can be selected from the group of esters of saturated and/or unsaturated, branched and/or unbranched alkanecarboxylic acids of chain length from 3 to 30 carbon atoms and saturated and/or unsaturated, branched and/or unbranched alcohols of chain length from 3 to 30 carbon atoms, and also from the group of esters of aromatic carboxylic acids and saturated and/or unsaturated, branched and/or unbranched alcohols of chain length from 3 to 30 carbon atoms. Such ester oils can then advantageously be selected from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, dicaprylyl carbonate (Cetiol CC) and cocoglycerides (Myritol 331), butylene glycol dicaprylate/dicaprate and dibutyl adipate, and synthetic, semisynthetic and natural mixtures of such esters, such as, e.g. jojoba oil.

Furthermore, one or more oil components can advantageously be selected from the group of branched and unbranched hydrocarbons and hydrocarbon waxes, silicone oils, dialkyl ethers, the group of saturated or unsaturated, branched or unbranched alcohols.

Any mixtures of such oil and wax components are also to be used advantageously for the purposes of the present invention. It may also, if appropriate, be advantageous to use waxes, for example cetyl palmitate, as the sole lipid component of the oil phase.

According to the invention, the oil component is advantageously selected from the group 2-ethylhexyl isostearate, octyidodecanol, isotridecyl isononanoate, isoeicosane, 2-ethylhexyl cocoate, C₁₂₋₁₅-alkyl benzoate, caprylic/capric triglyceride, dicaprylyl ether.

According to the invention, mixtures of C₁2-₁₅-alkyl benzoate and 2-ethylhexyl isostearate, mixtures of C₁₂₋₁₅-alkyl benzoate and isotridecyl isononanoate, and mixtures of C₁₂₋₁₅-alkyl benzoate, 2-ethylhexyl isostearate and isotridecyl isononanoate are advantageous.

According to the invention, as oils with a polarity of from 5 to 50 mN/m, particular preference is given to using fatty acid triglycerides, in particular soybean oil and/or almond oil.

Of the hydrocarbons, paraffin oil, cycloparaffin, squalane, squalene, polydecene and in particular (optionally hydrogenated) polyisobutenes are to be used advantageously for the purposes of the present invention. Such hydrogenated polyisobutenes are described, for example, in the unpublished German patent application with the application number DE 102005022021.5, to which reference is hereby made in its entirety.

In a preferred embodiment of the invention, the preparations according to the invention comprise polyisobutene and/or reactive polyisobutene which is used as described above for the production of the amphiphilic block copolymers a), where the polyisobutene used is sometimes not reactive and/or the reactive polyisobutene is not reacted as described above according to one of steps i) to xi), i.e. the reactive double bond remains intact. It is particularly advantageous to use such mixtures of unreactive polyisobutene, reactive polyisobutene, reactive polyisobutene reacted according to one of steps i) to xi) and amphiphilic block copolymers a) which are formed in the production of the amphiphilic block copolymers a) for producing the preparations according to the invention.

Such mixtures are commercially available, for example, as Glissopal®, Hyvis® or Napvis®.

In addition, the oil phase can advantageously be selected from the group of Guerbet alcohols. Guerbet alcohols are named after Marcel Guerbet who described their production for the first time. They are formed according to the reaction equation

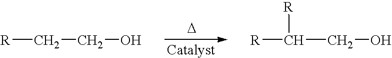

by oxidation of an alcohol to an aldehyde, by aldol condensation of the aldehyde, cleaving off of water from the aldol and hydrogenation of the allyl aldehyde. Guerbet alcohols are liquid even at low temperatures and cause virtually no skin irritations. They can advantageously be used as fatting, superfatting and also refatting constituents in cosmetic preparations.

The use of Guerbet alcohols in cosmetics is known per se. Such species are then characterized in most cases by the structure

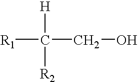

Here, R₁ and R₂ are usually unbranched alkyl radicals.

According to the invention, the Guerbet alcohol or alcohols are advantageously selected from the group where

R₁=propyl, butyl, pentyl, hexyl, heptyl or octyl and

R₂=hexyl, heptyl, octyl, nonyl, decyl, undecyl, dodecyl, tridecyl or tetradecyl.

Guerbet alcohols preferred according to the invention are 2-butyloctanol (commercially available, for example, as Isofol®12 (Condea)) and 2-hexyldecanol (commercially available, for example, as Isofol®16 (Condea)).

Mixtures of Guerbet alcohols according to the invention are also to be used advantageously according to the invention, such as, for example, mixtures of 2-butyloctanol and 2-hexyldecanol (commercially available, for example, as Isofol®14 (Condea)).

Any mixtures of such oil and wax components are also to be used advantageously for the purposes of the present invention.

The oil component can advantageous also have a content of cyclic or linear silicone oils or consist entirely of such oils, although it is preferred to use an additional content of other oil phase components apart from silicone oil. Low molecular weight silicones or silicone oils are usually defined by the following general formula

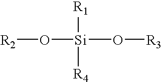

Higher molecular weight silicones or silicone oils are generally defined by the following general formula

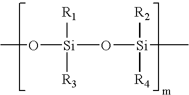

where the silicon atoms may be substituted by identical or different alkyl radicals and/or aryl radicals, which are represented here in general terms by the radicals R₁ to R₄. However, the number of different radicals is not necessarily limited to up to 4. m can here assume values of from 2 to 200 000.

Cyclic silicones to be used advantageously according to the invention are generally defined by the following general formula

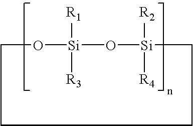

where the silicon atoms may be substituted by identical or different alkyl radicals and/or aryl radicals, which are shown here in general terms by the radicals R₁ to R₄. However, the number of different radicals is not necessarily limited to up to 4. n here can assume values from 3/2 to 20. Fractional values for n take into consideration that uneven numbers of siloxyl groups may be present in the cycle.

Phenyltrimethicone is advantageously selected as silicone oil. Other silicone oils, for example dimethicone, hexamethylcyclotrisiloxane, phenyldimethicone, cyclomethicone (e.g. decamethylcyclopentasiloxane), hexamethylcyclotrisiloxane, polydimethylsiloxane, poly(methylphenylsiloxane), cetyldimethicone, behenoxydimethicone are also to be used advantageously for the purposes of the present invention. Mixtures of cyclomethicone and isotridecyl isononanoate, and also those of cyclomethicone and 2-ethylhexyl isostearate are also advantageous. However, it is also advantageous to choose silicone oils of similar constitution to the compounds described above whose organic side chains have been derivatized, for example polyethoxylated and/or polypropoxylated. These include, for example, polysiloxane polyalkyl-polyether copolymers, such as, for example, cetyl-dimethicone copolyol.

Cyclomethicone (octamethylcyclotetrasiloxane) is advantageously used as silicone oil to be used according to the invention.

Fat components and/or wax components to be used advantageously according to the invention can be selected from the group of vegetable waxes, animal waxes, mineral waxes and petrochemical waxes. For example, candelilla wax, carnauba wax, Japan wax, espartograss wax, cork wax, guaruma wax, rice germ oil wax, sugar cane wax, berry wax, ouricury wax, montan wax, jojoba wax, shea butter, beeswax, shellac waxes, spermaceti, lanolin (wool wax), uropygial grease, ceresin, ozokerite (earth wax), paraffin waxes and microwaxes.

Further advantageous fat components and/or wax components are chemically modified waxes and synthetic waxes, such as, for example, Syncrowax®HRC (glyceryl tribe-henate), and Syncrowax®AW 1 C (C₁₈₋₃₆-fatty acid), and montan ester waxes, sasol waxes, hydrogenated jojoba waxes, synthetic or modified beeswaxes (e.g. dimethicone copolyol beeswax and/or C₃₀₋₅₀-alkyl beeswax), cetyl ricinoleates, such as, for example, Tegosoft®CR, polyalkylene waxes, polyethylene glycol waxes, but also chemically modified fats, such as, for example, hydrogenated vegetable oils (for example hydrogenated castor oil and/or hydrogenated coconut fatty glycerides), triglycerides, such as, for example, hydrogenated soy glyceride, trihydroxystearin, fatty acids, fatty acid esters and glycol esters, such as, for example, C₂₀₋₄₀-alkyl stearate, C₂₀₋₄₀-alkylhydroxystearoyl stearate and/or glycol montanate. Also further advantageous are certain organosilicon compounds which have similar physical properties to the specified fat components and/or wax components, such as, for example, stearoxytrimethylsilane.

According to the invention, the fat components and/or wax components can be used either individually or as a mixture in the preparations.

Any mixtures of such oil components and wax components are also to be used advantageously for the purposes of the present invention.

The oil phase is advantageously selected from the group 2-ethylhexyl isostearate, octyidodecanol, isotridecyl isononanoate, butylene glycol dicaprylate/dicaprate, 2-ethylhexyl cocoate, C₁₂₋₁₅-alkyl benzoate, caprylic/capric acid triglyceride, dicaprylyl ether.

Mixtures of octyidodecanol, caprylic/capric acid triglyceride, dicaprylyl ether, dicaprylyl carbonate, cocoglycerides or mixtures of C₁₂₋₁₅-alkyl benzoate and 2-ethylhexyl isostearate, mixtures of C₁₂₋₁₅-alkyl benzoate and butylene glycol dicaprylate/dicaprate, and mixtures of C₁₂₋₁₅-alkyl benzoate, 2-ethylhexyl isostearate and isotridecyl isononanoate are particularly advantageous.

The oil component is furthermore advantageously selected from the group of phospholipids. The phospholipids are phosphoric acid esters of acylated glycerols. Of greatest importance among the phosphatidylcholines are, for example, the lecithins, which are characterized by the general structure

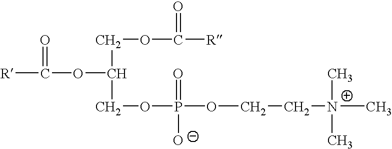

where R′ and R″ are typically unbranched aliphatic radicals having 15 or 17 carbon atoms and up to 4 cis double bonds.

According to the invention, Merkur Weissoel Pharma 40 from Merkur Vaseline, Shell Ondina® 917, Shell Ondina® 927, Shell Oil 4222, Shell Ondina®933 from Shell & DEA Oil, Pionier® 6301 S, Pionier® 2071 (Hansen & Rosenthal) can be used as paraffin oil advantageous according to the invention.

The content of the oils and/or fat phase c) is at most 50, preferably at most 30, further preferably at most 20% by weight, based on the total weight of the preparation.

Suitable cosmetically and pharmaceutically compatible oil and/or fat phases c) are described in Karl-Heinz Schrader, Grundlagen und Rezepturen der Kosmetika [Fundamentals and Formulations of Cosmetics], 2nd edition, Verlag Hüthig, Heidelberg, pp. 319-355, to which referenced is hereby made.

According to the invention, apart from the abovementioned substances, the preparations comprise further additives customary in cosmetics or dermatology.

Such further additives are, for example, UV photoprotective agents, antioxidants, refatting agents, superfatting agents, antiperspirants, perfume, dyes, antimicrobial substances, refatting agents, complexing agents and sequestrants, pearlizing agents, plant extracts, vitamins, active ingredients, conditioners, preservatives, bactericides, pigments which have a coloring effect, thickeners, softening, moisturizing and/or humectant substances, alcohols, polyols, polymers, organic acids, foam stabilizers, electrolytes, organic solvents or silicone derivatives.

With regard to the specified further ingredients known to the person skilled in the art for the preparations, reference may be made to “Kosmetik und Hygiene von Kopf bis Fuβ” [Cosmetics and hygiene from head to toe], ed. W. Umbach, 3rd edition, Wiley-VCH, 2004, pp. 123-128, to which reference is made at this point in its entirety.

Antiperspirants

By influencing the activity of the ecrine sweat glands, antiperspirants reduce the formation of perspiration and thus counteract armpit wetness and body odor. Aqueous or anhydrous formulations of antiperspirants typically comprise the following ingredients:
astringent active ingredients,
oil components,
nonionic emulsifiers,
coemulsifiers,
consistency regulators,
auxiliaries, such as, for example, thickeners or complexing agents and/or
nonaqueous solvents, such as, for example, ethanol, propylene glycol and/or glycerol.

Suitable astringent antiperspirant active ingredients are primarily salts of aluminum, of zirconium or of zinc. Such suitable antihydrotic active ingredients are, for example, aluminum chloride, aluminum chlorohydrate, aluminum dichlorohydrate, aluminum sesquichlorohydrate and complex compounds thereof, e.g. with propylene glycol-1,2, aluminum hydroxyallantoinate, aluminum chloride tartrate, aluminum zirconium trichlorohydrate, aluminum zirconium tetrachlorohydrate, aluminum zirconium pentachlorohydrate and complex compounds thereof, e.g. with amino acids such as glycine.

In addition, customary oil-soluble and water-soluble auxiliaries may be present in antiperspirants in smaller amounts.

Such oil-soluble auxiliaries may, for example, be:
antiinflammatory, skin-protecting or pleasant-smelling essential oils,
synthetic skin-protecting active ingredients and/or
oil-soluble perfume oils.

Customary water-soluble additives are, for example, preservatives, water-soluble fragrances, pH extenders, e.g. buffer mixtures, water-soluble thickeners, e.g. water-soluble natural or synthetic polymers, such as, for example, xanthan gum, hydroxyethylcellulose, polyvinylpyrrolidone or high molecular weight polyethylene oxides. Reference may also be made to the statements in “Kosmetik” [Cosmetics], editor W. Umbach, Thieme Verlag Stuttgart, 2nd edition 1995, pp. 372-376, to which reference is made at this point in its entirety.

Antidandruff Agents

Antidandruff agents which can be used are Octopirox® (1-hydroxy-4-methyl-6-(2,4,4-trimethylpentyl)-2-(1H)-pyridonemonoethanolamine salt), Baypival®, piroctone olamine, Ketoconazole®, (4-acetyl-1-(-4-[2-(2.4-dichlorophenyl)-2-(1H-imidazol-1-ylmethyl)-1,3-dioxylan-c-4-ylmethoxyphenyl)piperazine, selenium disulfide, sulfur colloidal, sulfur polyethylene glycol sorbitan monooleate, sulfur rizinol polyethoxylate, sulfur tar distillates, salicylic acid (e.g. in combination with hexachlorophene), undexylenic acid monoethanolamide sulfosuccinate Na salt, Lamepon® UD (protein-undecylenic acid condensate, zinc pyrethione, aluminum pyrithione and magnesium pyrithione/dipyrithione magnesium sulfate.

Ethoxylated Glycerol Fatty Acid Esters

Further ingredients to be used advantageously for the preparations according to the invention are ethoxylated oils selected from the group of ethoxylated glycerol fatty acid esters, particularly preferably PEG-10 olive oil glycerides, PEG-11 avocado oil glycerides, PEG-11 cocoa butter glycerides, PEG-13 sunflower oil glycerides, PEG-15 glyceryl isostearate, PEG-9 coconut fatty acid glycerides, PEG-54 hydrogenated castor oil, PEG-7 hydrogenated castor oil, PEG-60 hydrogenated castor oil, jojoba oil ethoxylate (PEG-26 jojoba fatty acids, PEG-26 jojoba alcohol), glycereth-5 cocoate, PEG-9 coconut fatty acid glycerides, PEG-7 glyceryl cocoate, PEG-45 palm kernel oil glycerides, PEG-35 castor oil, olive oil-PEG-7 ester, PEG-6 caprylic acid/capric acid glycerides, PEG-10 olive oil glycerides, PEG-13 sunflower oil glycerides, PEG-7 hydrogenated castor oil, hydrogenated palm kernel oil glyceride-PEG-6 ester, PEG-20 corn oil glycerides, PEG-18 glyceryl oleate cocoate, PEG40 hydrogenated castor oil, PEG-40 castor oil, PEG-60 hydrogenated castor oil, PEG-60 corn oil glycerides, PEG-54 hydrogenated castor oil, PEG-45 palm kernel oil glycerides, PEG-80 glyceryl cocoate, PEG-60 almond oil glycerides, PEG-60 “Evening Primrose” glycerides, PEG-200 hydrogenated glyceryl palmate, PEG-90 glyceryl isostearate.

Preferred ethoxylated oils are PEG-7 glyceryl cocoate, PEG-9 cocoglycerides, PEG-40 hydrogenated castor oil, PEG-200 hydrogenated glyceryl palmate.

Ethoxylated glycerol fatty acid esters are used in aqueous cosmetic preparations for various purposes. Glycerol fatty acid esters with low degrees of ethoxylation (3-12 ethylene oxide units) usually serve as refatting agents for improving the feel on the skin after drying, glycerol fatty acid esters with a degree of ethoxylation of about 30-50 serve as solubility promoters for nonpolar substances such as perfume oils. Glycerol fatty acid esters with high degrees of ethoxylation are used as thickeners. It is common to all of these substances that they produce a special feel on the skin upon application when diluted with water.

Conditioners

In a preferred embodiment of the invention, the preparations also comprise conditioners. Conditioners preferred according to the invention are, for example, all compounds which are listed in the International Cosmetic Ingredient Dictionary and Handbook (volume 4, editor: R. C. Pepe, J. A. Wenninger, G. N. McEwen, The Cosmetic, Toiletry, and Fragrance Association, 9th edition, 2002) under Section 4 under the keywords Hair Conditioning Agents, Humectants, Skin-Conditioning Agents, Skin-Conditioning Agents-Emollient, Skin-Conditioning Agents-Humectant, Skin-Conditioning Agents-Miscellaneous, Skin-Conditioning Agents-Occlusive und Skin Protectants, and all compounds listed in EP-A 934 956 (pp.11-13) under “water soluble conditioning agent” and “oil soluble conditioning agent”. Further advantageous conditioners are, for example, the compounds referred to according to the INCl as Polyquaternium (in particular Polyquaternium-1 to Polyquaternium-56).

Suitable conditioners include, for example, also polymeric quaternary ammonium compounds, cationic cellulose derivatives, chitosan derivatives and polysaccharides. Conditioners advantageous according to the invention can be selected here from the compounds given in table 1 below.

TABLE 1
Conditioners to be used advantageously

| | | | Example |
|---|---|---|---|
| INCI name | CAS Number | Type of polymer | (tradename) |
| Polyquaternium-2 | CAS 63451-27-4 | Urea, N,N′-bis[3- | Mirapol ® A-15 |
| | | (dimethylamino)propyl]-, | |
| | | polymer with 1,1′-oxybis | |
| | | (2-chloroethane) | |
| Polyquaternium-5 | CAS 26006-22-4 | Acrylamide, β-methacryloxy- | |
| | | ethyltriethylammonium | |
| | | methosulfate | |
| Polyquaternium-6 | CAS 26062-79-3 | N,N-dimethyl-N-2- | Merquat ® 100 |
| | | propenyl-2-propenaminium | |
| | | chloride | |
| Polyquaternium-7 | CAS 26590-05-6 | N,N-dimethyl-N-2-propenyl-2- | Merquat ® S |
| | | propenaminium chloride, | |
| | | 2-propenamide | |
| Polyquaternium-10 | CAS 53568-66-4, | Quaternary ammonium | Celquat ® SC-230M, |
| | 55353-19-0, 54351-50- | salt of hydroxy- | Polymer JR 400 |
| | 7, 68610-92-4, 81859- | ethylcellulose | |
| | 24-7 | | |
| Polyquaternium-11 | CAS 53633-54-8 | Vinylpyrrolidone/dimethyl- | Gafquat ® 755N |
| | | aminoethyl methacrylate | |
| | | copolymer/diethyl sulfate | |
| | | reaction product | |
| Polyquaternium-16 | CAS 29297-55-0 | Vinylpyrrolidone/ | Luviquat ® HM552 |
| | | vinylimidazolinum | |
| | | methochloride copolymer | |
| Polyquaternium-17 | CAS 90624-75-2 | | Mirapol ® AD-1 |
| Polyquaternium-19 | CAS 110736-85-1 | Quaternized water- | |
| | | soluble polyvinyl alcohol | |
| Polyquaternium-20 | CAS 110736-86-2 | Quaternized polyvinyl | |
| | | octadecyl ether | |
| | | dispersible in water | |
| Polyquaternium-21 | | Polysiloxane-polydi- | Abil ® B 9905 |
| | | methyldimethylammonium | |
| | | acetate copolymer | |
| Polyquaternium-22 | CAS 53694-17-0 | Dimethyldiallylammonium | Merquat ® 280 |
| | | chloride/acrylic | |
| | | acid copolymer | |
| Polyquaternium-24 | CAS 107987-23-5 | Polymeric quaternary | Quartisoft ® LM-200 |
| | | ammonium salt of | |
| | | hydroxyethylcellulose | |
| Polyquaternium-28 | CAS 131954-48-8 | Vinylpyrrolidone/methacryl- | Gafquat ® HS-100 |
| | | amidopropyltrimethyl- | |
| | | ammonium chloride copolymer | |
| Polyquaternium-29 | CAS 92091-36-6, | Chitosan which has been | Lexquat ® CH |
| | 148880-30-2 | reacted with propylene oxide | |
| | | and quaternized with | |
| | | epichlorohydrin | |
| Polyquaternium-31 | CAS 136505-02-7, | Polymeric, quaternary ammonium | Hypan ® QT 100 |
| | 139767-67-7 | salt which has been produced by | |
| | | reacting DMAPA acrylate/acrylic | |
| | | acid/acrylonitrogens copolymer | |
| | | and diethyl sulfate | |
| Polyquaternium-32 | CAS 35429-19-7 | N,N,N-trimethyl-2-{[β2-methyl- | |
| | | 1-oxo-2-propenyl)oxy]- | |
| | | ethanaminium chloride, | |
| | | polymer with 2-propenamide | |
| Polyquaternium-37 | CAS 26161-33-1 | | |
| Polyquaternium-44 | | Copolymeric quaternary | |
| | | ammonium salt of | |
| | | vinylpyrrolidone and | |
| | | quaternized imidazoline | |

Further conditioners advantageous according to the invention are cellulose derivatives and quaternized guar gum derivatives, in particular guar hydroxypropylammonium chloride (e.g. Jaguar Excel®, Jaguar C 162® (Rhodia), CAS 65497-29-2, CAS 39421-75-5). Nonionic poly-N-vinylpyrrolidone/polyvinyl acetate copolymers (e.g. Luviskol®VA 64 (BASF)), anionic acrylate copolymers (e.g. Luviflex®Soft (BASF)), and/or amphoteric amide/acrylate/methacrylate copolymers (e.g. Amphomer® (National Starch)) can also be used advantageously according to the invention as conditioners. Further possible conditioners are quaternized silicones.

Antioxidants

In a preferred embodiment, the cosmetic preparations comprise antioxidants. According to the invention, antioxidants which can be used are all antioxidants suitable or customary for cosmetic and/or dermatological applications.

The antioxidants are advantageously selected from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, γ-lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof), and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerated doses (e.g. pmol to μmol/kg), also (metal) chelating agents (e.g. α-hydroxyfatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, furfurylidene sorbitol and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of benzoic resin, rutinic acid and derivatives thereof, α-glycosylrutin, ferulic acid, furfurylidene glucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) suitable according to the invention of these specified active ingredients.

The amount of the abovementioned antioxidants (one or more compounds) in the preparations is preferably 0.001 to 30% by weight, particularly preferably 0.05 to 20% by weight, in particular 0.1 to 10% by weight, based on the total weight of the preparation.

If vitamin E and/or derivatives thereof are the antioxidant or anti oxidants, it is advantageous to provide these in concentrations of from 0.001 to 10% by weight, based on the total weight of the preparation.

If vitamin A or vitamin A derivatives, or carotenes or derivatives thereof are the antioxidant or the antioxidants, it is advantageous to provide these in concentrations of from 0.001 to 10% by weight, based on the total weight of the preparation.

(Co) Emulsifiers

The preparations according to the invention can also comprise further (co)emulsifiers different from b). Suitable as such are, for example, nonionogenic surfactants from at least one of the following groups:

Addition products of from 2 to 30 mol of ethylene oxide and/or 0 to 5 mol of propylene oxide onto linear fatty alcohols having 8 to 22 carbon atoms, onto fatty acids having 12 to 22 carbon atoms, onto alkylphenols having 8 to 15 carbon atoms in the alkyl group, and alkylamines having 8 to 22 carbon atoms in the alkyl radical;

addition products of from 1 to 15 mol of ethylene oxide onto castor oil and/or hydrogenated castor oil;

addition products of from 15 to 60 mol of ethylene oxide onto castor oil and/or hydrogenated castor oil;

partial esters of glyerol and/or sorbitan with unsaturated, linear or saturated, branched fatty acids having 12 to 22 carbon atoms and/or hydroxycarboxylic acids having 3 to 18 carbon atoms, and adducts thereof having 1 to 30 mol of ethylene oxide;

partial esters of polyglycerol (average degree of self-condensation 2 to 8), polyethylene glycol (molecular weight 400 to 5000), trimethylolpropane, pentaerythritol, sugar alcohols (e.g. sorbitol), alkyl glucosides (e.g. methyl glucoside, butyl glucoside, lauryl glucoside), and polyglucosides (e.g. cellulose) with saturated and/or unsaturated, linear or branched fatty acids having 12 to 22 carbon atoms and/or hydroxycarboxylic acids having 3 to 18 carbon atoms, and adducts thereof having 1 to 30 mol of ethylene oxide;

mixed esters of pentaerythritol, fatty acids, citric acid and fatty alcohol as in DE 1165574 C and/or mixed esters of fatty acids having 6 to 22 carbon atoms, methylglucose and polyols, preferably glycerol or polyglycerol.

Mono-, di- and trialkyl phosphates, and mono-, di- and/or tri-PEG alkyl phosphates and salts thereof;

wool wax alcohols;

polysiloxane-polyalkyl-polyether copolymers or corresponding derivatives;

polyalkylene glycols, and

glycerol carbonate.

The addition products of ethylene oxide and/or of propylene oxide onto fatty alcohols, fatty acids, alkylphenols or onto castor oil are known, commercially available products. These are homolog mixtures whose average degree of alkoxylation corresponds to the ratio of the quantitative amounts of ethylene oxide and/or propylene oxide and substrate with which the addition reaction is carried out. C_{12/18}-fatty acid mono- and -diesters of addition products of ethylene oxide onto glycerol are known from DE 2024051 C as refatting agent for cosmetic preparations.

Typical examples of suitable partial glycerides are hydroxystearic acid monoglyceride, hydroxystearic acid di-glyceride, isostearic acid monoglyceride, isostearic acid diglyceride, oleic acid monoglyceride, oleic acid diglyceride, ricinoleic acid moglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric diglyceride, malic acid monoglyceride, malic acid diglyceride, and technical-grade mixtures thereof which can also comprise small amounts of triglyceride from the production process as minor component. Addition products of from 1 to 30, preferably 5 to 10, mol of ethylene oxide onto the specified partial glycerides are likewise suitable.

Sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaieate, sorbitan sesquimaleate, sorbitan dimaleate, sorbitan trimaleate, and technical-grade mixtures thereof. Addition products of from 1 to 30, preferably 5 to 10, mol of ethylene oxide onto the specified sorbitan esters are likewise suitable.

Typical examples of suitable polyglycerol esters are polyglyceryl-2 dipolyhydroxystearate (Dehymuls® PGPH), polyglycerol-3 diisostearate (Lameform® TGI), polyglyceryl-4 isostearate (Isolan® GI 34), polyglyceryl-3 oleate, diisostearoyl polyglyceryl-3 diisostearate (Isolan^{B} PDI), polyglyceryl-3 methylglucose distearate (Tego Care® 450), polyglyceryl-3 beeswax (Cera Bellina®), polyglyceryl-4 caprate (Polyglycerol Caprate T2010/90), polyglyceryl-3 cetyl ether (Chimexane® NL), polyglyceryl-3 distearate (Cremophor® GS 32) and polyglyceryl polyricinoleate (Admul® WOL 1403) polyglyceryl dimerate isostearate, and mixtures thereof.

Examples of further suitable polyol esters are the mono-, di- and triesters, if appropriate reacted with 1 to 30 mol of ethylene oxide, of trimethylolpropane or pentaerythritol with lauric acid, coconut fatty acid, tallow fatty acid, palmitic acid, stearic acid, oleic acid, behenic acid and the like.

Furthermore, emulsifiers which can be used are zwitterionic surfactants. Zwitterionic surfactants is the term used to refer to those surface-active compounds which carry at least one quaternary ammonium group and at least one carboxylate and one sulfonate group in the molecule. Particularly suitable zwitterionic surfactants are the so-called betaines, such as the N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxymethyl-3-hydroxyethylimidazolines having in each case 8 to 18 carbon atoms in the alkyl or acyl group, and cocoacylaminoethyl hydroxyethylcarboxymethyl glycinate. The fatty acid amide derivative known under the CTFA name Cocamidopropyl Betaine is particularly preferred.

Likewise suitable emulsifiers are ampholytic surfactants. Ampholytic surfactants are understood as meaning those surface-active compounds which, apart from a C_{8/18}-alkyl or -acyl group in the molecule, comprise at least one free amino group and at least one —COOH or —SO₃H group and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids having in each case about 8 to 18 carbon atoms in the alkyl group. Particularly preferred ampholytic surfactants are N-cocoalkylaminopropionate, cocoacylaminoethylaminopropionate and C_{12/18}-acylsarcosine.

Finally, cationic surfactants are also suitable as emulsifiers, those of the ester quat type, preferably methyl-quaternized difatty acid triethanolamine ester salts, being particularly preferred.

Apart from the amphiphilic block copolymers a), the preparations according to the invention must comprise no further (co)emulsifiers.

UV Filter Substances

In a preferred embodiment, the preparations according to the invention comprise oil-soluble and/or water-soluble UVA and/or UVB filters. The preparations advantageously comprise substances which absorb UV radiation in the UVB region and substances which absorb UV radiation in the UVA region, where the total amount of the filter substances is, for example, 0.1 to 30% by weight, preferably 0.5 to 20% by weight, in particular 1 to 15% by weight, based on the total weight of the preparations, in order to provide cosmetic preparations which protect the skin from the entire range of ultraviolet radiation.

The greatest part of the photoprotective agents in the cosmetic or dermatological preparations serving to protect the human epidermis consists of compounds which absorb UV light in the UV-B region. For example, the fraction of the UV-A absorbers to be used according to the invention is, for example, 10 to 90% by weight, preferably 20 to 50% by weight, based on the total amount of substances absorbing UV-B and UV-A.

The UVB filters may be oil-soluble or water-soluble. Advantageous UVB filter substances are, for example:

benzimidazolesulfonic acid derivatives, such as, for example, 2-phenylbenzimidazole-5-sulfonic acid and salts thereof

benzotriazole derivatives, such as, for example, 2,2′-methylenebis(6-(2H-benzotriazol-2-yl)-4-(1,1,3,3-tetramethylbutyl)phenol)

4-aminobenzoic acid derivatives, preferably 2-ethylhexyl 4-(dimethylamino)benzoate, amyl 4-(dimethylamino)benzoate;

esters of benzalmalonic acid, preferably di(2-ethylhexyl) 4-methoxybenzalmalonate;

esters of cinnamic acid, preferably 2-ethylhexyl 4-methoxycinnamate, isopentyl 4-methoxycinnamate;

derivatives of benzophenone, preferably 2-hydroxy-4-methoxybenzophenone, 2-hydroxy-4-methoxy-4′-methylbenzophenone, 2,2′-dihydroxy-4-methoxybenzophenone;

methylidenecamphor derivatives, preferably 4-methylbenzylidenecamphor, benzylidenecamphor;

triazine derivatives, preferably tris(2-ethylhexyl) 4,4′,4″-(1,3,5-triazine-2,4,6-triylimino)-trisbenzoate [INCl: Diethylhexyl Butamido Triazine, UVA-Sorb® HEB (Sigma 3V)] and 2,4,6-tris-[anilino(p-carbo-2′-ethyl-1′-hexyloxy)]-1,3,5-triazine [INCl: Octyl Triazone, UVINUL®T 150 (BASF)].

Water-soluble UVB filter substances to be used advantageously are, for example, sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and salts thereof.

UVA filters to be used advantageously are, for example:

1,4-phenylenedimethinecamphorsulfonic acid derivatives, such as, for example, 3,3′-(1,4-phenylenedimethine)bis(7,7-dimethyl-2-oxobicyclo[2.2.1]heptane-1-methamsulfonic acid and its salts

1,3,5-triazine derivatives, such as 2,4-bis{[(2-ethylhexyloxy)-2-hydroxy)phenyl}-6-(4-methoxyphenyl)-1,3,5)triazine (e.g. Tinosorb®S (Ciba))

dibenzoylmethane derivatives, preferably 4-isopropyldibenzoylmethane, 4-(tert-butyl)-4′-methoxydibenzoyl methane

benzoxazole derivatives, for example 2,4-bis[4-[5-(1,1-dimethylpropyl)benzoxazol-2-yl]phenylimino]-6-[(2-ethylexyl)imino]-1,3,5-triazine (CAS No. 288254-16-0, Uvasorb®K2A (3V Sigma))

hydroxybenzophenones, for example hexyl 2-(4′-diethylamino-2′-hydoxybenzoyl)-benzoate (also: aminobenzophenone) (Uvinul®A Plus (BASF))

Furthermore, according to the invention, it may, if appropriate, be advantageous to provide preparations with further UVA and/or UVB filters, for example certain salicylic acid derivatives, such as 4-isopropylbenzyl salicylate, 2-ethylhexyl salicylate, octyl salicylate, homomenthyl salicylate.

The total amount of salicylic acid derivatives in the cosmetic or dermatological preparations is advantageously selected from the range 0.1-15.0, preferably 0.3-10.0% by weight, based on the total weight of the preparations. A further photoprotective filter to be used advantageously according to the invention is ethylhexyl 2-cyano-3,3-diphenylacrylate (Octocrylen, Uvinul®N 539 (BASF)).

The table below lists some of the photoprotective filters suitable for use in the preparations according to the invention:

For example, UV photoprotective filters to be mentioned are

| | | CAS No. |
|---|---|---|
| No. | Substance | (=acid) |
| 1 | 4-aminobenzoic acid | 150-13-0 |
| 2 | 3-(4′-trimethylammonium)benzylidenebornan-2-one | 52793-97-2 |
| | methylsulfate | |
| 3 | 3,3,5-trimethylcyclohexyl salicylate | 118-56-9 |
| | (homosalate) | |
| 4 | 2-hydroxy-4-methoxybenzophenone | 131-57-7 |
| | (oxybenzone) | |
| 5 | 2-phenylbenzimidazole-5-sulfonic acid and its potassium, | 27503-81-7 |
| | sodium and triethanolamine salts | |
| 6 | 3,3′-(1,4-phenylenedimethine)bis(7,7-dimethyl- | 90457-82-2 |
| | 2-oxobicyclo[2.2.1]heptane-1-methanesulfonic acid) and its | |
| | salts | |
| 7 | polyethoxyethyl 4-bis(polyethoxy)aminobenzoate | 113010-52-9 |
| 8 | 2-ethylhexyl 4-dimethylaminobenzoate | 21245-02-3 |
| 9 | 2-ethylhexyl salicylate | 118-60-5 |
| 10 | 2-isoamyl 4-methoxycinnamate | 71617-10-2 |
| 11 | 2-ethylhexyl 4-methoxycinnamate | 5466-77-3 |
| 12 | 2-hydroxy-4-methoxybenzophenone-5-sulfonic acid | 4065-45-6 |
| | (sulisobenzone) and the sodium salt | |
| 13 | 3-(4′-sulfobenzylidene)bornan-2-one and salts | 58030-58-6 |
| 14 | 3-benzylidenebornan-2-one | 16087-24-8 |
| 15 | 1-(4′-isopropylphenyl)-3-phenylpropane-1,3-dione | 63260-25-9 |
| 16 | 4-isopropylbenzyl salicylate | 94134-93-7 |
| 17 | 3-imidazol-4-ylacrylic acid and its ethyl ester | 104-98-3 |
| 18 | ethyl 2-cyano-3,3-diphenylacrylate | 5232-99-5 |
| 19 | 2′-ethylhexyl 2-cyano-3,3-diphenylacrylate | 6197-30-4 |
| 20 | menthyl o-aminobenzoate or: | 134-09-8 |
| | 5-methyl-2-(1-methylethyl)-2-aminobenzoate | |
| 21 | glyceryl p-aminobenzoate or: | 136-44-7 |
| | 1-glyceryl 4-aminobenzoate | |
| 22 | 2,2′-dihydroxy-4-methoxybenzophenone (dioxybenzone) | 131-53-3 |
| 23 | 2-hydroxy-4-methoxy-4-methylbenzophenone | 1641-17-4 |
| | (mexenone) | |
| 24 | triethanolamine salicylate | 2174-16-5 |
| 25 | dimethoxyphenylglyoxalic acid or: | 4732-70-1 |
| | 3,4-dimethoxyphenylglyoxalacidic sodium | |
| 26 | 3-(4′-sulfobenzylidene)bornan-2-one and its salts | 56039-58-8 |
| 27 | 4-tert-butyl-4′-methoxydibenzoylmethane | 70356-09-1 |
| 28 | 2,2′,4,4′-tetrahydroxybenzophenone | 131-55-5 |
| 29 | 2,2′-methylenebis[6-(2H-benzotriazol-2-yl)-4-(1,1,3,3- | 103597-45-1 |
| | tetramethylbutyl)phenol] | |
| 30 | 2,2′-(1,4-phenylene)bis-1H-benzimidazole-4,6- | 180898-37-7 |
| | disulfonic acid, Na salt | |
| 31 | 2,4-bis[4-(2-ethylhexyloxy)-2-hydroxy]phenyl- | 187393-00-6 |
| | 6-(4-methoxyphenyl)(1,3,5)-triazine | |
| 32 | 3-(4-methylbenzylidene)camphor | 36861-47-9 |
| 33 | polyethoxyethyl 4-bis(polyethoxy)paraaminobenzoate | 113010-52-9 |
| 34 | 2,4-dihydroxybenzophenone | 131-56-6 |
| 35 | 2,2′-dihydroxy-4,4′-dimethoxybenzophenone-5,5′- | 3121-60-6 |
| | disodium sulfonate | |
| 36 | benzoic acid, 2-[4-(diethylamino)-2-hydroxybenzoyl]-, hexyl ester | 302776-68-7 |
| 37 | 2-(2H-benzotriazol-2-yl)-4-methyl-6-[2-methyl-3-[1,3,3,3- | 155633-54-8 |
| | tetramethyl-1-[(trimethylsilyl)oxy]disiloxanyl]propyl]phenol | |
| 38 | 1,1-[(2,2′-dimethylpropoxy)carbonyl]-4,4-diphenyl-1,3-butadiene | 363602-15-7 |

According to the invention, polymeric or polymer-bound filter substances can also be used.

Metal oxides, such as titanium dioxide or zinc oxide, are used widely in sunscreen compositions. Their effect is essentially based on reflection, scattering and absorption of the harmful UV radiation and essentially depends on the primary particle size of the metal oxides. Furthermore, the cosmetic or dermatological preparations according to the invention can advantageously comprise inorganic pigments based on metal oxides and/or other metal compounds that are insoluble or sparingly soluble in water, selected from the group of the oxides of zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals, and mixtures of such oxides. They are particularly preferably pigments based on ZnO.

The inorganic pigments may be present here in coated form, i.e. have been surface-treated. This surface treatment can, for example, consist in providing the pigments with a thin hydrophobic layer in a method known per se, as described in DE-A-33 14 742.

Photoprotective agents suitable for use in the preparations according to the invention are the compounds specified in EP-A 1 084 696 in paragraphs [0036] to [0053], to which reference is made at this point in its entirety. Of suitability for the use according to the invention are all UV photoprotective filters which are specified in annex 7 (to Section 3b) of the German Cosmetics Ordinance under “Ultraviolet filters for cosmetic compositions”.

The list of specified UV photoprotective filters which can be used in the preparations according to the invention is not exhaustive.

Active Ingredients

It has been found that highly diverse active ingredients of varying solubility can be homogeneously incorporated into the cosmetic preparations according to the invention.

According to the invention, the active ingredients (one or more compounds) can advantageously be selected from the group consisting of acetylsalicylic acid, atropine, azulene, hydrocortisone and derivatives thereof, e.g. hydrocortisone-17 valerate, vitamins B and D series, in particular vitamin B₁, vitamin B₁₂, vitamin D, vitamin A or derivatives thereof, such as retinyl palmitate, vitamin E or derivatives thereof, such as, for example, tocopheryl acetate, vitamin C and derivatives thereof, such as, for example, ascorbyl glucoside, but also niacinamide, panthenol, bisabolol, polydocanol, unsaturated fatty acids, such as, for example, the essential fatty acids (usually referred to as vitamin F), in particular γ-linolenic acid, oleic acid, eicosapentanoic acid, docosahexanoic acid and derivatives thereof, chloramphenicol, caffeine, prostaglandins, thymol, camphor, squalene, extracts or other products of vegetable and animal origin, for example evening primrose oil, borage oil or currant seed oil, fish oils, cod liver oil, but also ceramides and ceramide-like compounds, frankincense extract, green tea extract, water lily extract, liquorice extract, hamamelis, antidandruff active ingredients (e.g. selenium disulfide, zinc pyrithione, piroctone, olamine, climbazole, octopirox, polydocanol and combinations thereof), complex active ingredients, such as, for example, those from γ-oryzanol and calcium salts, such as calcium panthotenate, calcium chloride, calcium acetate.

It is also advantageous to choose the active ingredients from the group of refatting substances, for example purcellin oil, Eucerit® and Neocerit®.

In addition, the active ingredient or active ingredients are particularly advantageously selected from the group of NO synthase inhibitors, particularly if the preparations according to the invention are to serve for the treatment and prophylaxis of the symptoms of intrinsic and/or extrinsic skin aging and also for the treatment and prophylaxis of the harmful effects of ultraviolet radiation on the skin and the hair. A preferred NO synthase inhibitor is nitroarginine.

The active ingredient or active ingredients are further advantageously selected from the group comprising catechins and bile acid esters of catechins and aqueous or organic extracts of plants and parts of plants which have a content of catechins or bile acid esters of catechins, such as, for example, the leaves of the Theaceae plant family, in particular of the species Camellia sinensis (green tea). Their typical ingredients (e.g. polyphenols or catechins, caffeine, vitamins, sugars, minerals, amino acids, lipids) are particularly advantageous.

Catechins constitute a group of compounds which are to be regarded as hydrogenated flavones or anthocyanidins and are derivatives of “catechins” (catechol, 3,3′,4′,5,7-flavanepentaol, 2-(3,4-dihydroxyphenyl)chromane-3,5,7-triol). Epicatechin ((2R,3R)-3,3′,4′,5,7-flavanepentaol) is also an advantageous active ingredient for the purposes of the present invention.

Also advantageous are plant extracts with a content of catechins, in particular extracts of green tea, such as, for example, extracts of leaves of the plants of the species Camellia spec., very particularly of the tea varieties Camellia sinenis, C. assamica, C. taliensis and C. inawadiensis and hybrids of these with, for example, Camellia japonica.

Preferred active ingredients are also polyphenols or catechins from the group (−)-catechin, (+)-catechin, (−)-catechin gallate, (−)-gallocatechin gallate, (+)-epicatechin, (−)-epicatechin, (−)-epicatechin gallate, (−)-epigallocatechin, (−)-epigallocatechin gallate.

Flavone and its derivatives (often also collectively called “flavones”) are also advantageous active ingredients for the purposes of the present invention. They are characterized by the following basic structure (substitution positions given):

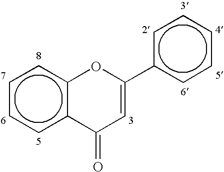

Some of the more important flavones which can also preferably be used in preparations according to the invention are listed in table 2 below.

TABLE 2
Table 2: Flavones

| | OH— substitution positions | | | | | | | |
|---|---|---|---|---|---|---|---|---|
| | 3 | 5 | 7 | 8 | 2′ | 3′ | 4′ | 5′ |
| Flavone | − | − | − | − | − | − | − | − |
| Flavonol | + | − | − | − | − | − | − | − |
| Chrysin | − | + | + | − | − | − | − | − |
| Galangin | + | + | + | − | − | − | − | − |
| Apigenin | − | + | + | − | − | − | + | − |
| Fisetin | + | − | + | − | − | + | + | − |
| Luteolin | − | + | + | − | − | + | + | − |
| Kaempferol | + | + | + | − | − | − | + | − |
| Quercetin | + | + | + | − | − | + | + | − |
| Morin | + | + | + | − | + | − | + | − |
| Robinetin | + | − | + | − | − | + | + | + |
| Gossypetin | + | + | + | + | − | + | + | − |
| Myricetin | + | + | + | − | − | + | + | + |

Headings Table 2 OH substitution positions
left-hand column reads
Flavone - then as German for next 6 lines then Kaempferol

In nature, flavones usually occur in glycosylated form.

According to the invention, the flavonoids are preferably selected from the group of substances of the general formula

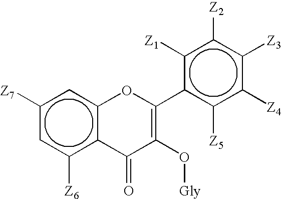

where Z₁ to Z₇, independently of one another, are selected from the group H, OH, alkoxy and hydroxyalkoxy, where the alkoxy or hydroxyalkoxy groups may be branched or unbranched and have 1 to 18 carbon atoms, and where Gly is selected from the group of mono- and oligoglycoside radicals.

However, according to the invention, the flavonoids can also be selected advantageously from the group of substances of the general formula

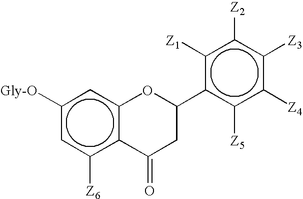

where Z₁ to Z₆, independently of one another, are as selected from the group H, OH, alkoxy and hydroxyalkoxy, where the alkoxy or hydroxyalkoxy groups may be branched or unbranched and have 1 to 18 carbon atoms, and where Gly is selected from the group of mono- and oligoglycoside radicals.

Preferably, such structures can be selected from the group of substances of the general formula

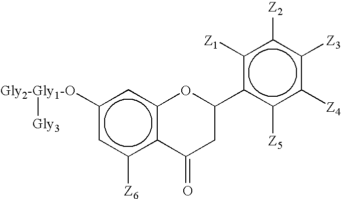

where Z₁ to Z₆, independently of one another, are as mentioned above and Gly₁, Gly₂ and Gly₃, independently of one another, are monoglycoside radicals or oligoglycoside radicals. Gly₂ and Gly₃ can also, individually or together, represent saturations by hydrogen atoms.

Preferably, Gly₁, Gly₂ and Gly₃, independently of one another, are selected from the group of hexosyl radicals, in particular the rhamnosyl radicals and glucosyl radicals. However, other hexosyl radicals, for example allosyl, altrosyl, galactosyl, gulosyl, idosyl, mannosyl and talosyl, are also, if appropriate, to be used advantageously.

It may also be advantageous according to the invention to use pentosyl radicals.

Advantageously, Z₁ to Z₅, independently of one another, are selected from the group H, OH, methoxy, ethoxy and 2-hydroxyethoxy, and the flavone glycosides correspond to the general structural formula

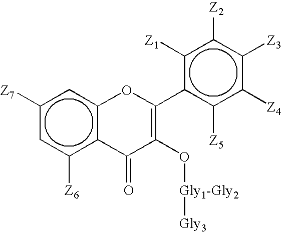

The flavone glycosides are particularly advantageously selected from the group which is represented by the following structure

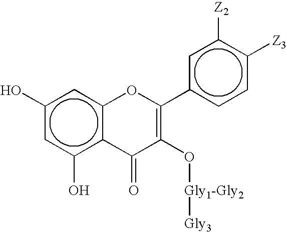

where Gly₁, Gly₂ and Gly₃, independently of one another, are monoglycoside radicals or oligoglycoside radicals. Gly₂ and Gly₃ can also, individually or together, represent saturations by hydrogen atoms.

Preferably, Gly₁, Gly₂ and Gly₃, independently of one another, are preferably selected from the group of hexosyl radicals, in particular the rhamnosyl radicals and glucosyl radicals. However, other hexosyl radicals, for example allosyl, altrosyl, galactosyl, gulosyl, idosyl, mannosyl and talosyl, are also, if appropriate, to be used advantageously.

According to the invention, it may also be advantageous to use pentosyl radicals.

For the purposes of the present invention, it is particularly advantageous to select the flavone glycoside or the flavone glycosides from the group α-glucosylrutin, α-glucosylmyricetin, α-glucosylisoquercitrin, a-glucosylisoquercetin and α-glucosylquercitrin.

Further advantageous active ingredients are sericoside, pyridoxol, vitamin K, biotin and aroma substances. Furthermore, the active ingredients (one or more compounds) can also very advantageously be selected from the group of hydrophilic active ingredients, in particular from the following group: α-hydroxy acids, such as lactic acid or salicylic acid, or salts thereof, such as, for example, Na lactate, Ca lactate, TEA lactate, urea, allantoin, serine, sorbitol, glycerol, milk proteins, panthenol, chitosan.

The list of the specified active ingredients or active ingredient combinations which can be used in the preparations according to the invention is not of course intended to be limiting. The active ingredients can be used individually or in any combinations with one another.

The amount of such active ingredients (one or more compounds) in the preparations according to the invention is preferably 0.001 to 30% by weight, particularly preferably 0.05 to 20% by weight, in particular 1 to 10% by weight, based on the total weight of the preparation.

The specified and further active ingredients which can be used in the preparations according to the invention are given in DE 103 18 526 A1 on pages 12 to 17, to which reference is made at this point in its entirety.

Pearlescent Waxes

Suitable pearlescent waxes for the use in the preparations according to the invention are, for example: alkylene glycol esters, specifically ethylene glycol distearate; fatty acid alkanolamides, specifically coconut fatty acid diethanolamide; partial glycerides, specifically stearic acid monoglyceride; esters of polybasic, optionally hydroxy-substituted carboxylic acids with fatty alcohols having 6 to 22 carbon atoms, specifically long-chain esters of tartaric acid; fatty substances, such as, for example, fatty alcohols, fatty ketones, fatty aldehydes, fatty ethers and fatty carbonates, which in total have at least 24 carbon atoms, specifically laurone and distearyl ether; fatty acids, such as, stearic acid, hydroxystearic acid or behenic acid, ring-opening products of olefin epoxides having 12 to 22 carbon atoms with fatty alcohols having 12 to 22 carbon atoms and/or polyols having 2 to 15 carbon atoms and 2 to 10 hydroxyl groups, and mixtures thereof.

Furthermore, the preparations according to the invention can comprise glitter substances and/or other effect substances (e.g. color streaks).

Enzyme Inhibitors

Suitable enzyme inhibitors are, for example, esterase inhibitors. These are preferably trialkyl citrates, such as trimethyl citrate, tripropyl citrate, triisopropyl citrate, tributyl citrate and, in particular triethyl citrate (Hydagen®CAT). The substances inhibit the enzyme activity and thereby reduce the odor formation. Further substances which are suitable as esterase inhibitors are sterol sulfates or phosphates, such as, for example, lanosterol, cholesterol, campesterol, stigmasterol and citosterol sulfate or phosphate, dicarboxylic acids and esters thereof, such as, for example, glutaric acid, monoethyl glutarate, diethyl glutarate, adipic acid, monoethyl adipate, diethyl adipate, malonic acid and diethyl malonate, hydroxycarboxylic acids and esters thereof, such as, for example, citric acid, malic acid, tartaric acid or diethyl tartrate, and zinc glycinate.

Dyes

Dyes which can be used are the subtances approved and suitable for cosmetic, dermatological or pharmaceutical purposes, as listed, for example, in the publication “Kosmetische Färbemittel” [Cosmetic colorants] of the Farbstoffkommission der Deutschen Forschungsgemeinschaft [Dyes Commission of the German Research Society], Verlag Chemie, Weinheim, 1984, pp. 81-106. These dyes are usually used in concentrations of from 0.001 to 0.1 by weight, based on the total mixture.

Film Formers

Customary film formers are, for example, chitosan, microcrystalline chitosan, quaternized chitosan, polyvinylpyrrolidone, vinylpyrrolidone-vinyl acetate copolymers, polymers of the acrylic acid series, quaternary cellulose derivatives, collagen, hyaluronic acid and salts thereof and similar compounds.

Gel Formers

Gel formers which can be used are all gel formers customary in cosmetics. These include lightly crosslinked polyacrylic acid, for example Carbomer (INCl), cellulose derivatives, e.g. hydroxypropylcellulose, hydroxyethylcellulose, cationically modified celluloses, polysaccharides, e.g. xanthum gum, caprylic/capric triglycerides, sodium acrylate copolymer, polyquaternium-32 (and) Paraffinum Liquidum (INCl), sodium acrylates copolymer (and) Paraffinum Liquidum (and) PPG-1 trideceth-6, acrylamidopropyltrimonium chloride/acrylamide copolymer, steareth-10 allyl ether acrylates copolymer, polyquaternium-37 (and) Paraffinum Liquidum (and) PPG-1 trideceth-6, polyquaternium 37 (and) propylene glycol dicaprate dicaprylate (and) PPG-1 trideceth-6, polyquaternium-7, polyquaternium-44.

Consistency Regulators

Suitable consistency regulators are primarily fatty alcohols or hydroxyfatty alcohols having 12 to 22 and preferably 16 to 18 carbon atoms and also partial glycerides, fatty acids or hydroxyfatty acids. Preference is given to a combination of these substances with alkyl oligoglucosides and/or fatty acid N-methylglucamides of equal chain length and/or polyglycerol poly-12-hydroxystearates. Suitable thickeners are, for example, polysaccharides, in particular xanthan gum, guar-guar, agar-agar, alginates and tyloses, carboxymethylcellulose and hydroxyethylcellulose, also relatively high molecular weight polyethylene glycol mono- and diesters of fatty acids, polyacrylates (e.g. Carbopol® from Goodrich or Synthalen® from Sigma), polyacrylamides, polyvinyl alcohol and polyvinylpyrrolidone, surfactants, such as, for example, ethoxylated fatty acid glycerides, esters of fatty acids with polyols, such as, for example, pentaerythritol or trimethylolpropane, fatty alcohol ethoxylates with a narrowed homolog distribution or alkyl oligoglucosides, and electrolytes such as sodium chloride and ammonium chloride.

Thickeners

The cosmetic preparations according to the invention can also comprise thickeners. Suitable thickeners for the preparations according to the invention are crosslinked polyacrylic acids and derivatives thereof, polysaccharides, such as xanthan gum, guar-guar, agar-agar, alginates or tyloses, cellulose derivatives, e.g. carboxymethylcellulose or hydroxycarboxymethylcellulose, also relatively high molecular weight polyethylene glycol mono- and diesters of fatty acids, fatty alcohols, monoglycerides and fatty acids, polyvinyl alcohol and polyvinylpyrrolidone.

Suitable thickeners are also polyacrylates such as Carbopol® (Noveon), Ultrez® (Noveon), Luvigel® EM (BASF), Capigel®98 (Seppic), Synthalene® (Sigma), the Aculyn® grades from Rohm and Haas, such as Aculyn® 22 (copolymer of acrylates and methacrylic acid ethoxylates with stearyl radical (20 EO units)) and Aculyn® 28 (copolymer of acrylates and methacrylic acid ethoxylates with behenyl radical (25 EO units)).

Suitable thickeners are also, for example, aerosil grades (hydrophilic silicas), polyacrylamides, polyvinyl alcohol and polyvinylpyrrolidone, surfactants, such as, for example, ethoxylated fatty acid glycerols, esters of fatty acids with polyols, such as, for example, pentaerythritol or trimethylolpropane, fatty alcohol ethoxylates with a narrowed homolog distribution or alkyl oligoglucosides, and electrolytes such as sodium chloride and ammonium chloride.

Odor Absorbers and Perfume Oils

Suitable odor absorbers are substances which can absorb and largely hold onto odor-forming compounds. They lower the partial pressure of the individual components and thus also reduce their rate of spread. It is important that perfumes here have to remain unaffected. Odor absorbers have no effectiveness against bacteria. As main constituent, they comprise, for example, a complex zinc salt of ricinoleic acid or special, largely odor-neutral fragrances which are known to the person skilled in the art as “fixative”, such as, for example, extracts of labdanum or styrax or certain abietic acid derivatives.

Functioning as odor-masking agents are fragrances or perfume oils which, in addition to their function as odor-masking agent, impart their particular scent note to the deodorants. Perfume oils which may be mentioned are, for example, mixtures of natural and synthetic fragrances. Natural fragrances are extracts of flowers, stems and leaves, fruits, fruit peels, roots, woods, herbs and grasses, needles and branches, and resins and balsams. Also suitable are animal raw materials, such as, for example, civet and castoreum. Typical synthetic fragrance compounds are products of the ester, ether, aldehyde, ketone, alcohol and hydrocarbon types. Fragrance compounds of the ester type are, for example, benzyl acetate, p-tert-butylcyclohexyl acetate, linalyl acetate, phenylethyl acetate, linalyl benzoate, benzyl formate, allylcyclohexyl propionate, styrallyl propionate and benzyl salicylate. The ethers include, for example, benzyl ethyl ether, the aldehydes, for example, the linear alkanals having 8 to 18 carbon atoms, citrate, citronellal, citronellyloxyacetaldehyde, cyclamenaldehyde, hydroxycitronellal, lilial and bourgeonal, the ketones, for example, the ionones and methyl cedryl ketone, the alcohols anethol, citronellol, eugenol, isoeugenol, geraniol, linalool, phenylethyl alcohol and terpineol, the hydrocarbons include primarily the terpenes and balsams.

However, preference is given to using mixtures of different fragrances which together produce a pleasant scent note. Essential oils of lower volatility, which are mostly used as aroma components, are also suitable as perfume oils, e.g. sage oil, camomile oil, clove oil, melissa oil, mint oil, cinnamon leaf oil, linden blossom oil, juniper berry oil, vetiver oil, olibanum oil, galbanum oil, labdanum oil and lavandin oil. Preferably, bergamot oil, dihydromyrcenol, lilial, lyral, citronellol, phenylethyl alcohol, alpha-hexylcinnamaldehyde, geraniol, benzyl acetone, cyclamenaldehyde, linalool, Boisambrene Forte, ambroxan, indole, hedione, sandelice, lemon oil, mandarin oil, orange oil, allyl amyl glycolate, cyclovertal, lavandin oil, clary sage oil, beta-damascone, geranium oil Bourbon, cyclohexyl salicylate, Vertofix®Coeur, Iso-E-Super®, Fixolide®NP, evernyl, iraldein gamma, phenylacetic acid, geranyl acetate, benzyl acetate, rose oxide, romilat, irotyl and floramat, alone or in mixtures.

Hydrotopes

To improve the flow behavior, hydrotropes, such as, for example, ethanol, isopropyl alcohol, or polyols can also be used. Polyols which are suitable here preferably have 2 to 15 carbon atoms and at least two hydroxyl groups.

Typical examples are glycerol;

alkylene glycols, such as, for example, ethylene glycol, diethylene glycol, propylene glycol, butylene glycol, hexylene glycol, and polyethylene glycols with an average molecular weight of from 100 to 1000 g/mol; technical-grade oligoglycerol mixtures with a degree of self-condensation of from 1.5 to 10, such as, for example, technical-grade diglycerol mixtures with a diglycerol content of from 40 to 50% by weight.

methylol compounds, such as, in particular, trimethylolethane, trimethylolpropane,

trimethylolbutane, pentaerythritol and dipentaerythritol;

low alkyl glucosides, in particular those having 1 to 8 carbon atoms in the alkyl radical, such as, for example, methyl glucoside and butyl glucoside;

sugar alcohols having 5 to 12 carbon atoms, such as, for example, sorbitol or mannitol;

sugars having 5 to 12 carbon atoms, such as, for example, glucose or sucrose;

amino sugars, such as, for example, glucamine.

Suitable insect repellants are N,N-diethyl-m-toluamide, 1,2-pentanediol or ethyl butylacetylaminopropionate, suitable self-tanning agents are dihydroxyacetone. Suitable tyrosine inhibitors, which prevent the formation of melanine and are used in depigmentation compositions, are, for example, arbutin, kojic acid, coumaric acid and ascorbic acid (vitamin C).

Antibacterial Agents

Suitable antibacterial agents are in principle all substances effective against Gram-positive bacteria, such as, for example, 4-hydroxybenzoic acid and its salts and esters, N-(4-chlorophenyl)-N′-(3,4-dichlorophenyl)urea, 2,4,4′-trichloro-2′-hydroxydiphenyl ether(triclosan), 4-chloro-3,5-dimethylphenol, 2,2′-methylenebis(G-bromo-4-chlorophenol), 3-methyl-4-(1-methylethyl)phenol, 2-benzyl-4-chlorophenol, 3-(4-chlorophenoxy)-1,2-propanediol, 3-iodo-2-propynyl butylcarbamate, chlorhexidine, 3,4,4′-trichlorocarbanilide (TTC), antibacterial fragrances, thymol, thyme oil, eugenol, clove oil, menthol, mint oil, farnesol, phenoxyethanol, glycerol monolaurate (GML), diglycerol monocaprate (DMC), N-alkylsalicylamides, such as, for example, n-octylsalicylamide or n-decylsalicylamide.

The antibacterially effective substances are generally used in concentrations of from about 0.1 to 0.3% by weight.

Preservatives

In one embodiment of the invention, the cosmetic preparations according to the invention can also comprise preservatives. Preparations with high water contents must be reliably protected against the build-up of germs. The most important preservatives used for this purpose are urea condensates, p-hydroxybenzoic acid esters, the combination of phenoxyethanol with methyldibromoglutaronitrile and acid preservatives with benzoic acid, salicylic acid and sorbic acid.

Preparations with high fractions of surfactants or polyols and low water contents can also be formulated free from preservatives.

The preparations according to the invention can advantageously comprise one or more preservatives. Advantageous preservatives for the purposes of the present invention are, for example, formaldehyde donors (such as, for example, DMDM hydantoin, which is commercially available, for example, under the tradename Glydant® (Lonza), iodopropyl butylcarbamates (e.g. Glycacil-L®, Glycacil-S® (Lonza), Dekaben®LMB (Jan Dekker)), parabens (p-hydroxybenzoic acid alkyl esters, such as, for example, methyl, ethyl, propyl and/or butyl paraben), dehydroacetic acid (Euxyl® K 702 (Schülke&Mayr), phenoxyethanol, ethanol, benzoic acid. So-called preservation aids, such as, for example, octoxyglycerol, glycines, soya etc., are also advantageously used.

The table below gives an overview of customary preservatives which may also be present in the cosmetic preparations according to the invention.

| | |
|---|---|
| E 200 | sorbic acid |
| E 201 | sodium sorbate |
| E 202 | potassium sorbate |
| E 203 | calcium sorbate |
| E 210 | benzoic acid |
| E 211 | sodium benzoate |
| E 212 | potassium benzoate |
| E 213 | calcium benzoate |
| E 214 | ethyl p-hydroxybenzoate |
| E 215 | ethyl p-hydroxybenzoate Na salt |
| E 216 | n-propyl p-hydroxybenzoate |
| E 217 | n-propyl p-hydroxybenzoate Na salt |
| E 218 | methyl p-hydroxybenzoate |
| E 219 | methyl p-hydroxybenzoate Na salt |
| E 220 | sulfur dioxide |
| E 221 | sodium sulfite |
| E 222 | sodium hydrogensulfite |
| E 223 | sodium disulfite |
| E 224 | potassium disulfite |
| E 226 | calcium sulfite |
| E 227 | calcium hydrogensulfite |
| E 228 | potassium hydrogensulfite |
| E 230 | biphenyl (diphenyl) |
| E 231 | orthophenylphenol |
| E 232 | sodium orthophenyl phenoxide |
| E 233 | thiabendazole |
| E 235 | natamycin |
| E 236 | formic acid |
| E 237 | sodium formate |
| E 238 | calcium formate |
| E 239 | hexamethylenetetramine |
| E 249 | potassium nitrite |
| E 250 | sodium nitrite |
| E 251 | sodium nitrate |
| E 252 | potassium nitrate |
| E 280 | propionic acid |
| E 281 | sodium propionate |
| E 282 | calcium propionate |
| E 283 | potassium propionate |
| E 290 | carbon dioxide |

Also advantageous are the preservatives or preservative aids customary in cosmetics, such as dibromodicyanobutane (2-bromo-2-bromomethylglutarodinitrile), phenoxyethanol, 3-iodo-2-propynyl butylcarbamate, 2-bromo-2-nitropropane-1,3-diol, imidazolidinylurea, 5-chloro-2-methyl-4-isothiazolin-3-one, 2-chloroacetamide, benzalkonium chloride, benzyl alcohol, salicylic acid and salicylates.

It is particularly preferred if iodopropyl butylcarbamates, parabens(methyl, ethyl, propyl and/or butylparaben) and/or phenoxyethanol are used as preservatives. Suitable preservatives are generally the further classes of substances listed in Annex 6, Part A and B of the Cosmetics Ordinance.

According to the invention, preservatives are present in a total concentration of at most 2% by weight, preferably at most 1.5% by weight and particularly preferably at most 1% by weight, based on the total weight of the preparation.

Complexing Agents

Since the raw materials and also the preparations themselves are produced predominantly in steel apparatuses, the end products can comprise iron (ions) in trace amounts. In order to prevent these impurities adversely affecting product quality as a result of reactions with dyes and perfume oil constituents, complexing agents such as salts of ethylenediaminetetraacetic acid, of nitrilotriacetic acid, of iminodisuccinic acid or phosphates are added.

Pigments

In a preferred embodiment, the preparations according to the invention, in particular the hair and skin cosmetic preparations, comprise at least one pigment.

The pigments are present in the product mass in undissolved form and may be present in an amount of from 0.01 to 25% by weight, particularly preferably from 5 to 15% by weight. The preferred particle size is 1 to 200 μm, in particular 3 to 150 μm, particularly preferably 10 to 100 μm. The pigments are colorants that are virtually insoluble in the application medium and may be inorganic or organic. Inorganic-organic mixed pigments are also possible. Preference is given to inorganic pigments. The advantage of inorganic pigments is their excellent resistance to light, weather and temperature. The inorganic pigments may be of natural origin, for example produced from chalk, ocher, umbra, green earth, burnt siena or graphite. The pigments may be white pigments, such as, for example, titanium dioxide or zinc oxide, black pigments, such as, for example, iron oxide black, colored pigments, such as, for example, ultramarine or iron oxide red, luster pigments, metal effect pigments, pearlescent pigments, and fluorescent or phosporescent pigments, where preferably at least one pigment is a colored, nonwhite pigment.

Metal oxides, hydroxides and oxide hydrates, mixed phase pigments, sulfur-containing silicates, metal sulfides, complex metal cyanides, metal sulfates, chromates and molybdates, and the metals themselves (bronze pigments) are suitable. In particular, titanium dioxide (Cl 77891), black iron oxide (Cl 77499), yellow iron oxide (Cl 77492), red and brown iron oxide (Cl 77491 ), manganese violet (Cl 77742), ultramarine (sodium aluminum sulfosilicates, Cl 77007, Pigment Blue 29), chromium oxide hydrate (C177289), iron blue (ferric ferrocyanide, Cl7751 0), carmine (cochineal) are suitable.

Particular preference is given to pearlescent and colored pigments based on mica which are coated with a metal oxide or a metal oxychloride, such as titanium dioxide or bismuth oxychloride, and, if appropriate, further color-imparting substances, such as iron oxides, iron blue, ultramarine, carmine etc., and where the color can be determined by varying the layer thickness. Such pigments are sold, for example, under the tradenames Rona®, Colorona®, Dichrona® and Timiron® by Merck, Germany.

Organic pigments are, for example, the natural pigments sepia, gamboge, bone charcoal, Cassel brown, indigo, chlorophyll and other plant pigments. Synthetic organic pigments are, for example, azo pigments, anthraquinoids, indigoids, dioxazine, quinacridone, phthalocyanine, isoindolinone, perylene and perinone, metal complex, alkali blue and diketopyrrolopyrrole pigments.

In one embodiment, the preparation according to the invention comprises 0.01 to 10% by weight, particularly preferably from 0.05 to 5% by weight, of at least one particulate substance. Suitable substances are, for example, substances which are solid at room temperature (25° C.) and are present in the form of particles. For example, silica, silicates, aluminates, clay earths, mica, salts, in particular inorganic metal salts, metal oxides, e.g. titanium dioxide, minerals and polymer particles, are suitable.

The particles are present in the preparation in undissolved, preferably stably dispersed form, and, following application to the application surface and evaporation of the solvent, can be deposited in solid form.

Preferred particulate substances are silica (silica gel, silicon dioxide) and metal salts, in particular inorganic metal salts, where silica is particularly preferred. Metal salts are, for example, alkali metal or alkaline earth metal halides, such as sodium chloride or potassium chloride; alkali metal or alkaline earth metal sulfates, such as sodium sulfate or magnesium sulfate.

Polymers

To achieve certain advantageous effects or actions, the cosmetic preparations according to the invention can further comprise additional polymers.

Suitable polymers are, for example, cationic polymers with the INCl name Polyquaternium, e.g. copolymers of vinylpyrrolidone/N-vinylimidazolium salts (Luviquat® FC, Luviquat® HM, Luviquat® MS, Luviquat® Care, Luviquat® UltraCare, Luviquat® Supreme), copolymers of N-vinylpyrrolidone/dimethylaminoethyl methacrylate, quaternized with diethyl sulfate (Luviquat® PQ 11), copolymers of N-vinylcaprolactam/N-vinylpyrrolidone/N-vinylimidazolium salts (Luviquat® Hold); cationic cellulose derivatives (Polyquaternium-4 and -10), acrylamido copolymers (Polyquaternium-7) and chitosan. Suitable cationic (quaternized) polymers are also Merquat® (polymer based on dimethyldiallylammonium chloride), Gafquat® (quaternary polymers which are formed by reacting polyvinylpyrrolidone with quaternary ammonium compounds), polymer JR (hydroxyethylcellulose with cationic groups) and plant-based cationic polymers, e.g. guar polymers, such as the Jaguar® grades from Rhodia. Further suitable polymers are also neutral polymers, such as polyvinylpyrrolidones, copolymers of N-vinylpyrrolidone and vinyl acetate and/or vinyl propionate and/or stearyl(meth)acrylate, polysiloxanes, polyvinylcaprolactam and other copolymers with N-vinylpyrrolidone, polyethyleneimine and salts thereof, polyvinylamines and salts thereof, cellulose derivatives, polyaspartic acid salts and derivatives. These include, for example, Luviflex® Swing (partially saponified copolymer of polyvinyl acetate and polyethylene glycol, BASF) or Kollicoat® IR.

Suitable polymers are also the (meth)acrylic acid amide copolymers described in WO 03/092640, in particular those described as examples 1 to 50 (table 1, page 40 ff.) and examples 51 to 65 (table 2, page 43), to which reference is made at this point in its entirety.

Suitable polymers are also nonionic, water-soluble or water-dispersible polymers or oligomers, such as polyvinylcaprolactam, e.g. Luviskol® Plus (BASF), or polyvinylpyrrolidone and copolymers thereof, in particular with vinyl esters, such as vinyl acetate, e.g. Luviskol® VA 37 (BASF); polyamides, e.g. based on itaconic acid and aliphatic diamines, as are described, for example, in DE-A43 33 238.

Suitable polymers are also amphoteric or zwitterionic polymers, such as the octylacrylamide/methyl methacrylate/tert-butylaminoethyl methacrylate/2-hydroxypropyl methacrylate copolymers available under the names Amphomer® (National Starch), and zwitterionic polymers as are disclosed, for example, in the German patent applications DE 39 29 973, DE 21 50 557, DE 28 17 369 and DE 37 08 451. Acrylamidopropyltrimethylammonium chloride/acrylic acid or methacrylic acid copolymers and alkali metal and ammonium salts thereof are preferred zwitterionic polymers. Further suitable zwitterionic polymers are methacroylethylbetaine/methacrylate copolymers which are commercially available under the name Amersette® (AMERCHOL), and copolymers of hydroxyethyl methacrylate, methyl methacrylate, N,N-dimethylaminoethyl methacrylate and acrylic acid (Jordapon®).

Suitable polymers are also nonionic, siloxane-containing, water-soluble or -dispersible polymers, e.g. polyether siloxanes, such as Tegopren® (Goldschmidt) or Belsil® (Wacker).

Also suitable are, furthermore, biopolymers, i.e. polymers which are obtained from naturally renewable raw materials and are composed of natural monomer building blocks, e.g. cellulose derivatives, chitin derivatives, chitosan derivatives, DNA derivatives, hyaluronic acid derivatives and RNA derivatives.

Further preparations according to the invention comprise at least one further water-soluble polymer, in particular chitosans (poly(D-glucosamines)) of differing molecular weight and/or chitosan derivatives.

Anionic Polymers

Further polymers suitable for the preparations according to the invention are copolymers containing carboxylic acid groups. These are polyelectrolytes with a relatively large number of anionically dissociatable groups in the main chain and/or one side chain. They are capable of forming polyelectrolyte complexes (symplexes) with the copolymers A).

In a preferred embodiment, the polyelectrolyte complexes used in the compositions according to the invention have an excess of anionogenic/anionic groups.

Besides at least one of the abovementioned copolymers A), the polyelectrolyte complexes also comprise at least one acid-group-containing polymer.

The polyelectrolyte complexes preferably comprise copolymer(s) A) and acid-group-containing polymers in a quantitative weight ratio of from about 50:1 to 1:20, particularly preferably from 20:1 to 1:5.

Suitable polymers containing carboxylic acid groups are obtainable, for example, by free-radical polymerization of α,β-ethylenically unsaturated monomers. Use is made here of monomers m1) which comprise at least one free-radically polymerizable, α,β-ethylenically unsaturated double bond and at least one anionogenic and/or anionic group per molecule.

Suitable polymers containing carboxylic acid groups are also polyurethanes containing carboxylic acid groups. Preferably, the monomers are selected from monoethylenically unsaturated carboxylic acids, sulfonic acids, phosphonic acids and mixtures thereof.

The monomers m1) include monoethylenically unsaturated mono- and dicarboxylic acids having 3 to 25, preferably 3 to 6, carbon atoms, which can also be used in the form of their salts or anhydrides. Examples thereof are acrylic acid, methacrylic acid, ethacrylic acid, α-chloroacrylic acid, crotonic acid, maleic acid, maleic anhydride, itaconic acid, citraconic acid, mesaconic acid, glutaconic acid, aconitic acid and fumaric acid. Furthermore, the monomers include the half-esters of monoethylenically unsaturated dicarboxylic acids having 4 to 10, preferably 4 to 6, carbon atoms, e.g. of maleic acid, such as monomethyl maleate. The monomers also include monoethylenically unsaturated sulfonic acids and phosphonic acids, for example vinylsulfonic acid, allylsulfonic acid, sulfoethyl acrylate, sulfoethyl methacrylate, sulfopropyl acrylate, sulfopropyl methacrylate, 2-hydroxy-3-acryloxypropylsulfonic acid, 2-hydroxy-3-methacryloxypropylsulfonic acid, styrenesulfonic acid, 2-acrylamido-2-methylpropanesulfonic acid, vinylphosphonic acid and allylphosphonic acid. The monomers also include the salts of the abovementioned acids, in particular the sodium, potassium and ammonium salts, and the salts with the abovementioned amines. The monomers can be used as such or as mixtures with one another. The stated fractions by weight all refer to the acid form.

Preferably, the monomer ml) is selected from acrylic acid, methacrylic acid, ethacrylic acid, α-chloroacrylic acid, crotonic acid, maleic acid, maleic anhydride, fumaric acid, itaconic acid, citraconic acid, mesaconic acid, glutaconic acid, aconitic acid and mixtures thereof, particularly preferably acrylic acid, methacrylic acid and mixtures thereof.

The abovementioned monomers ml) can in each case be used individually or in the form of any mixtures.

Of suitability in principle as comonomers for producing the polymers containing carboxylic acid groups are the compounds a) to d) specified above as components of copolymer A) with the proviso that the molar fraction of anionogenic and anionic groups which the polymer containing carboxylic acid groups comprises in copolymerized form is greater than the molar fraction of cationogenic and cationic groups.

In a preferred embodiment, the polymers containing carboxylic acid groups comprise at least one monomer in copolymerized form which is selected from the abovementioned crosslinkers d). Reference is made to suitable and preferred crosslinkers d).

Furthermore, the polymers containing carboxylic acid groups preferably comprise at least one monomer m2) in copolymerized form which is selected from compounds of the general formula (VI)

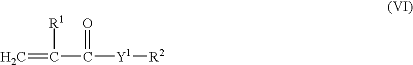

in which

R¹ is hydrogen or C₁-C₈-alkyl,

Y¹ is O, NH or NR³, and

R² and R³, independently of one another, are C₁-C₃₀-alkyl or C₅-C₈-cycloalkyl, where the alkyl groups may be interrupted by up to four nonadjacent heteroatoms or heteroatom-containing groups which are selected from O, S and NH. Preferably, R¹ in the formula VI is hydrogen or C₁-C₄-alkyl, in particular hydrogen, methyl or ethyl. Preferably, R² in the formula VI is C₁-C₈-alkyl, preferably methyl, ethyl, n-butyl, isobutyl, tert-butyl or a group of the formula —CH₂—CH₂—NH—C(CH₃)₃. If R³ is alkyl, then it is preferably C₁-C₄-alkyl, such as methyl, ethyl, n-propyl, n-butyl, isobutyl and tert-butyl.

Suitable monomers m2) are methyl(meth)acrylate, methyl ethacrylate, ethyl(meth)acrylate, ethyl ethacrylate, tert-butyl(meth)acrylate, tert-butyl ethacrylate, n-octyl(meth)acrylate, 1,1,3,3-tetramethylbutyl(meth)acrylate, ethylhexyl(meth)acrylate, n-nonyl(meth)acrylate, n-decyl(meth)acrylate, n-undecyl(meth)acrylate, tridecyl(meth)acrylate, myristyl(meth)acrylate, pentadecyl(meth)acrylate, palmityl(meth)acrylate, heptadecyl(meth)acrylate, nonadecyl(meth)acrylate, arrachinyl(meth)acrylate, behenyl(meth)acrylate, lignocerenyl(meth)acrylate, cerotinyl(meth)acrylate, melissinyl meth)acrylate, palmitoleinyl(meth)acrylate, oleyl(meth)acrylate, linolyl(meth)acrylate, linolenyl(meth)acrylate, stearyl(meth)acrylate, lauryl(meth)acrylate and mixtures thereof.

Suitable monomers m2) are also acrylamide, methacrylamide, N-methyl(meth)acrylamide, N-ethyl(meth)acrylamide, N-propyl(meth)acrylamide, N-(n-butyl)(meth)acrylamide, N-(tert-butyl)(meth)acrylamide, N,N-dimethyl(meth)acrylamide, N,N-diethyl(meth)acrylamide, piperidinyl(meth)acrylamide and morpholinyl(meth)acrylamide, N-(n-octyl)(meth)acrylamide, N-(1,1,3,3-tetramethylbutyl)(meth)acrylamide, N-ethylhexyl(meth)acrylamide, N-(n-nonyl)(meth)acrylamide, N-(n-decyl)(meth)acrylamide, N-(n-undecyl)(meth)acrylamide, N-tridecyl(meth)acrylamide, N-myristyl(meth)acrylamide, N-pentadecyl(meth)acrylamide, N-palmityl(meth)acrylamide, N-heptadecyl(meth)acrylamide, N-nonadecyl(meth)acrylamide, N-a rrachinyl(meth)acrylamide, N-behenyl(meth)acrylamide, N-lignocerenyl(meth)acrylamide, N-cerotinyl(meth)acrylamide, N-melissinyl(meth)acrylamide, N-palmitoleinyl(meth)acrylamide, N-oleyl(meth)acrylamide, N-linolyl(meth)acrylamide, N-linolenyl(meth)acrylamide, N-stearyl(meth)acrylamide and N-lauryl(meth)acrylamide.

Furthermore, the polymers containing carboxylic acid groups preferably comprise at least monomer m3) in copolymerized form which is selected from compounds of the general formula VII

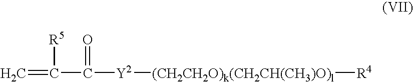

in which

the order of the alkylene oxide units is arbitrary,

k and l, independently of one another, are an integer from 0 to 1000, where the sum of k and l is at least 5,

R⁴ is hydrogen, C₁-C₃₀-alkyl or C₅-C₈-cycloalkyl,

R⁵ is hydrogen or C₁-C₈-alkyl,

Y² is O or NR⁶, where R⁶ is hydrogen, C₁-C₃₀-alkyl or C₅-C₈-cycloalkyl.

Preferably, in the formula VII, k is an integer from 1 to 500, in particular 3 to 250. Preferably,. I is an integer from 0 to 100. Preferably, R⁵ is hydrogen, methyl, ethyl, n-propyl, isopropyl, n-butyl, sec-butyl, tert-butyl, n-pentyl or n-hexyl, in particular hydrogen, methyl or ethyl. Preferably, R⁴ in the formula VII is hydrogen, methyl, ethyl, n-propyl, isopropyl, n-butyl, sec-butyl, n-pentyl, n-hexyl, octyl, 2-ethylhexyl, decyl, lauryl, palmityl or stearyl. Preferably, Y² in the formula VII is O or NH. Suitable polyether acrylates VII) are, for example, the polycondensation products of the abovementioned α,β-ethylenically unsaturated mono- and/or dicarboxylic acids and their acid chlorides, acid amides and anhydrides with polyetherols. Suitable polyetherols can be produced easily by reacting ethylene oxide, 1,2-propylene oxide and/or epichlorohydrin with a starter molecule, such as water or a short-chain alcohol R⁴—OH. The alkylene oxides can be used individually, alternately one after the other or as a mixture. The polyether acrylates VII) can be used on their own or in mixtures for producing the polymers used according to the invention. Suitable polyether acrylates II) are also urethane(meth)acrylates with alkylene oxide groups. Compounds of this type are described in DE 198 38 851 (component e2)), to which reference is made here in its entirety.

Anionic polymers preferred as polymers containing carboxylic acid groups are, for example, homopolymers and copolymers of acrylic acid and methacrylic acid and salts thereof. These also include crosslinked polymers of acrylic acid, as obtainable under the INCl name Carbomer. Such crosslinked homopolymers of acrylic acid are commercially available, for example, under the name Carbopol® from Noveon. Preference is also given to hydrophobically modified crosslinked polyacrylate polymers such as Carbopol® Ultrez 21 from Noveon.

Further examples of suitable anionic polymers are copolymers of acrylic acid and acrylamide and salts thereof; sodium salts of polyhydroxycarboxylic acids, water-soluble or water-dispersible polyesters, polyurethanes and polyureas. Particularly suitable polymers are copolymers of (meth)acrylic acid and polyether acrylates, where the polyether chain is terminated with a C₈-C₃₀-alkyl radical. These include, for example, acrylate/beheneth-25 methacrylate copolymers, which are available under the name Aculyn® from Rohm and Haas. Particularly suitable polymers are also copolymers of t-butyl acrylate, ethyl acrylate, methacrylic acid (e.g. Luvimer® 100P, Luvimer® Pro55), copolymers of ethyl acrylate and methacrylic acid (e.g. Luviumer® MAE), copolymers of N-tert-butylacrylamide, ethyl acrylate, acrylic acid (Ultrahold® 8, Ultrahold® Strong), copolymers of vinyl acetate, crotonic acid and, if appropriate, further vinyl esters (e.g. Luviset® grades), maleic anhydride copolymers, if appropriate reacted with alcohol, anionic polysiloxanes, e.g. carboxy-functional t-butyl acrylate, methacrylic acid (e.g. Luviskol® VBM), copolymers of acrylic acid and methacrylic acid with hydrophobic monomers, such as, for example, C₄-C₃₀-alkyl esters of meth(acrylic acid), C₄-C₃₀-alkylvinyl esters, C₄-C₃₀-alkyl vinyl ethers and hyaluronic acid. Examples of anionic polymers are also vinyl acetate/crotonic acid copolymers, as are commercially available, for example, under the names Resyn® (National Starch) and Gafset® (GAF), and vinylpyrrolidone/vinyl acrylate copolymers obtainable, for example, under the trade name Luviflex® (BASF). Further suitable polymers are the vinylpyrrolidone/acrylate terpolymer available under the name Luviflex® VBM-35 (BASF) and polyamides containing sodium sulfonate or polyesters containing sodium sulfonate.

Furthermore, the group of suitable anionic polymers comprises, by way of example, Balance® CR (National Starch; acrylate copolymer), Balance® 0/55 (National Starch; acrylate copolymer), Balance® 47 (National Starch; octylacrylamide/acrylates/butylaminoethyl methacrylates copolymer), Aquaflex® FX 64 (ISP; isobutylene/ethylmaleimide/hydroxyethylmaleimide copolymer), Aquaflex® SF-40 (ISP/National Starch; VP/vinyl caprolactam/DMAPA acrylates copolymer), Allianz®0 LT-120 (ISP/Rohm & Haas; Acrylate/C1-2 succinate/hydroxyacrylate copolymer), Aquarez® HS (Eastman; polyester-1), Diaformer® Z-400 (Clariant; methacryloylethylbetaine/methacrylate copolymer), Diaformer® Z-711 (Clariant; methacryloylethyl N-oxide/methacrylate copolymer), Diaformer® Z-712 (Clariant; methacryloylethyl N-oxide/methacrylate copolymer), Omnirez® 2000 (ISP; monoethyl ester of poly(methyl vinyl ether/maleic acid in ethanol), Amphomer® HC (National Starch; acrylate/octylacrylamide copolymer), Amphomer® 28-4910 (National Starch; octylacrylamide/acrylate/butylaminoethyl methacrylate copolymer), Advantage® HC 37 (ISP; terpolymer of vinylcaprolactam/vinylpyrrolidone/dimethylaminoethyl methacrylate), Advantage® LC55 and LC80 or LC A and LC E, Advantage® Plus (ISP; VA/butyl maleate/isobornyl acrylate copolymer), Aculyne® 258 (Rohm & Haas; acrylate/hydroxyester acrylate copolymer), Luviset® P.U.R. (BASF, polyurethane-1), Luviflex® Silk (BASF), Eastman® AQ 48 (Eastman), Styleze® CC-10 (ISP; VP/DMAPA acrylates copolymer), Styleze® 2000 (ISP; VP/acrylates/lauryl methacrylate copolymer), DynamX® (National Starch; polyurethane-14 AMP-acrylates copolymer), Resyn XP® (National Starch; acrylates/octylacrylamide copolymer), Fixomer® A-30 (Ondeo Nalco; polymethacrylic acid (and) acrylamidomethylpropanesulfonic acid), Fixate® G-100 (Noveon; AMP-acrylates/allyl methacrylates copolymer).

Suitable polymers containing carboxylic acid groups are also the terpolymers of vinylpyrrolidone, C₁-C₁₀-alkyl, cycloalkyl and aryl(meth)acrylates and acrylic acid described in U.S. Pat. No. 3,405,084. Suitable polymers containing carboxylic acid groups are furthermore the terpolymers of vinylpyrrolidone, tert-butyl(meth)acrylate and (meth)acrylic acid described in EP-A-0 257 444 and EP-A-0 480 280. Suitable polymers containing carboxylic acid groups are furthermore the copolymers described in DE-A-42 23 066 which comprise at least one (meth)acrylic acid ester, (meth)acrylic acid and N-vinylpyrrolidone and/or N-vinylcaprolactam in copolymerized form. Reference is made here to the disclosure of these documents in their entirety.

The abovementioned polymers containing carboxylic acid groups are produced by known methods, for example solution polymerization, precipitation polymerization, suspension polymerization or emulsion polymerization, as described above for the copolymers A).

Suitable polymers containing carboxylic acid groups are furthermore polyurethanes containing carboxylic acid groups.

EP-A-636361 discloses suitable block copolymers with polysiloxane blocks and polyurethane/polyurea blocks which have carboxylic acid and/or sulfonic acid groups. Suitable silicon-containing polyurethanes are also described in WO 97/25021 and EP-A-751 162.

Suitable polyurethanes are also described in DE-A42 25 045, to which reference is made here in its entirety.

The acid groups of the polymers containing carboxylic acid groups may be partially or completely neutralized. Then, at least some of the acid groups are in deprotonated form, where the counterions are preferably selected from alkali metal ions, such as Na⁺, K⁺, ammonium ions and organic derivatives thereof etc.

Propellants

If the preparations according to the invention are to be provided as aerosol spray, then propellants are necessary. Suitable propellants (propellant gases) are the customary propellants, such as n-propane, isopropane, n-butane, isobutane, 2,2-dimethylbutane, n-pentane, isopentane, dimethyl ether, difluoroethane, fluorotrichloromethane, dichlorodifluoromethane or dichlorotetrafluoroethane, HFC 152 A or mixtures thereof. In particular, hydrocarbons, in particular propane, n-butane, n-pentane and mixtures thereof, and dimethyl ether and difluoroethane are used. If appropriate, one or more of the specified chlorinated hydrocarbons are co-used in propellant mixtures, but only in small amounts, for example up to 20% by weight, based on the propellant mixture. The hair cosmetic preparations according to the invention are also suitable for pump spray preparations without the addition of propellants or also for aerosol sprays with customary pressurized gases, such as nitrogen, compressed air or carbon dioxide as propellant.

Swelling Agents

Swelling agents for aqueous phases which can be used are montmorillonites, clay mineral substances, pemulen, and alkyl-modified Carbopol® grades (Goodrich). Further suitable polymers and swelling agents can be found in the review by R. Lochhead in Cosm.Toil. 108, 95 (1993).

Stabilizers

Stabilizers which can be used are metal salts of fatty acids, such as, for example, magnesium, aluminum and/or zinc stearate or ricinoleate.

Surfactants

The preparations according to the invention can also comprise surfactants. Surfactants which may be used are anionic, cationic, nonionic and/or amphoteric surfactants.

For the purposes of the present invention, advantageous anionic surfactants are acylamino acids and salts thereof, such as
acyl glutamates, in particular sodium acyl glutamate
sarcosinates, for example myristoyl sarcosin, TEA-lauroyl sarcosinate, sodium lauroyl sarcosinate and sodium cocoyl sarcosinate,

sulfonic acids and salts thereof, such as
acyl isethionates, for example sodium or ammonium cocoyl isethionate
sulfosuccinates, for example dioctyl sodium sulfosuccinate, disodium laureth sulfosuccinate, disodium lauryl sulfosuccinate and disodium undecyleneamido MEA sulfosuccinate, disodium PEG-5 lauryl citrate sulfosuccinate and derivatives, and sulfuric acid esters, such as
alkyl ether sulfate, for example sodium, ammonium, magnesium, MIPA, TIPA laureth sulfate, sodium myreth sulfate and sodium C₁₂₋₁₃ pareth sulfate,
alkyl sulfates, for example sodium, ammonium and TEA lauryl sulfate.

Further advantageous anionic surfactants are
taurates, for example sodium lauroyl taurate and sodium methyl cocoyl taurate,
ether carboxylic acids, for example sodium laureth-13 carboxylate and sodium PEG-6 cocamide carboxylate, sodium PEG-7 olive oil carboxylate
phosphoric acid esters and salts, such as, for example, DEA oleth-10 phosphate and dilaureth-4 phosphate,
alkylsulfonates, for example sodium coconut monoglyceride sulfate, sodium C₁₂₋₁₄ olefinsulfonate, sodium lauryl sulfoacetate and magnesium PEG-3 cocamide sulfate,
acyl glutamates, such as di-TEA palmitoyl aspartate and sodium caprylic/capric glutamate,
acyl peptides, for example palmitoyl-hydrolyzed milk protein, sodium cocoyl hydrolyzed soya protein and sodium/potassium cocoyl hydrolyzed collagen

and carboxylic acids and derivatives, such as

for example laureic acid, aluminum stearate, magnesium alkanolate and zinc undecylenate, ester carboxylic acids, for example calcium stearoyl lactylate, laureth-6 citrate and sodium PEG-4 lauramidecarboxylate

alkylarylsulfonates.

Advantageous cationic surfactants for the purposes of the present invention are quaternary surfactants. Quaternary surfactants comprise at least one N atom which is covalently bonded to 4 alkyl or aryl groups. For example, alkylbetaine, alkylamidopropylbetaine and alkylamidopropylhydroxysultaine are advantageous.

Further advantageous cationic surfactants for the purposes of the present invention are also
alkylamines,
alkylimidazoles and
ethoxylated amines

and in particular salts thereof.

Advantageous amphoteric surfactants for the purposes of the present invention are acyl/dialkylethylenediamines, for example sodium acyl amphoacetate, disodium acyl amphodipropionate, disodium alkyl amphodiacetate, sodium acyl amphohydroxypropylsulfonate, disodium acyl amphodiacetate, sodium acyl amphopropionate, and N-coconut fatty acid amidoethyl-N-hydroxyethylglycinate sodium salts.

Further advantageous amphoteric surfactants are N-alkylamino acids, for example aminopropylalkylglutamide, alkylaminopropionic acid, sodium alkylimidodipropionate and lauroamphocarboxyglycinate.

Advantageous active nonionic surfactants for the purposes of the present invention are
alkanolamides, such as cocamides MEA/DEA/MIPA,
esters which are formed by esterifying carboxylic acids with ethylene oxide, glycerol, sorbitan or other alcohols,
ethers, for example ethoxylated alcohols, ethoxylated lanolin, ethoxylated polysiloxanes, propoxylatled POE ethers, alkyl polyglycosides, such as lauryl glucoside, decyl glycoside and cocoglycoside, glycosides with an HLB value of at least 20 (e.g. Belsil®SPG 128V (Wacker)).

Further advantageous nonionic surfactants are alcohols and amine oxides, such as cocoamidopropylamine oxide.

Among the alkyl ether sulfates, preference is given in particular to sodium alkyl ether sulfates based on di- or triethoxylated lauryl alcohol and myristyl alcohol. They surpass the alkyl sulfates considerably with regard to the insensitivity toward water hardness, the ability to be thickened, the low-temperature stability and, in particular, the skin and mucosa compatibility. Lauryl ether sulfate has better foam properties than myristyl ether sulfate, but is inferior to this in terms of mildness.

Alkyl ether carboxylates with an average and particularly with a high belong to the mildest surfactants overall, but exhibit a poor foaming and viscosity behavior. They are often used in combination with alkyl ether sulfates and amphoteric surfactants.

Sulfosuccinic acid esters (sulfosuccinates) are mild and highly foaming surfactants, but, on account of their poor ability to be thickened, are preferably used only together with other anionic and amphoteric surfactants and, on account of their low hydrolysis stability, are used preferably only in neutral or well buffered products.

Amidopropylbetaines have excellent skin and eye mucosa compatibility. In combination with anionic surfactants, their mildness can be synergistically improved. Preference is given to the use of cocamidopropylbetaine.

Amphoacetates/amphodiacetates have, as amphoteric surfactants, very good skin and mucosa compatibility and can have a conditioning effect and/or increase the care effect of additives. They are used similarly to the betaines for optimizing alkyl ether sulfate formulations. Sodium cocoamphoacetate and disodium cocoamphodiacetate are most preferred.

Alkyl polyglycosides are mild, have good universal properties, but are weakly foaming. For this reason, they are preferably used in combinations with anionic surfactants.

Furthermore, the use of a combination of anionic and/or amphoteric surfactants with one or more nonionic surfactants is advantageous.

Buffers

Buffers ensure the pH stability of aqueous compositions according to the invention. Preferably, citrate, lactate and phosphate buffers are used.

Solubility Promoters

Solubility promoters are used in order to bring care oils or perfume oils clearly into solution and to keep them clearly in solution even at low temperature. The most common solubility promoters are ethoxylated nonionic surfactants, e.g. hydrogenated and ethoxylated castor oils.

Superfatting Agents

Superfatting agents which can be used are substances such as, for example, lanolin and lecithin and polyethoxylated or acylated lanolin and lecithin derivatives, polyol fatty acid esters, monoglycerides and fatty acid alkanolamides, the latter also serving as foam stabilizers.

Self-Tanning Products

Standard commercial self-tanning products are generally O/W emulsions. In these, the water phase is stabilized by emulsifiers customary in cosmetics. A disadvantage is the required additional stabilization by carbomers. Their use in conjunction with self-tanning agents, in particular with dihydroxyacetone (DHA), leads, as a result of a chemical reaction, to a yellowish discoloration of the preparation and to odor impairments. One alternative to the use of carbomers is the use of xanthan gum. Although in this case stable products are obtained, an unpleasant sticky feel on the skin often has to be accepted.

A further object of the present invention was therefore to provide self-tanning products which do not have the abovementioned disadvantages.

Surprisingly, this object was achieved by preparations according to the invention which comprise one or more self-tanning substances.

Accordingly, the invention also further provides cosmetic preparations according to the invention which furthermore comprise one or more self-tanning substances and, if appropriate, further cosmetic and/or dermatological active ingredients, auxiliaries and additives.

The preparations according to the invention may be present and used in various forms. Thus, for example, they may be an emulsion of the oil-in-water (O/W) type or a multiple emulsion, for example of the water-in-oil-in-water(W/O/W) type. Emulsifier-free formulations, such as hydrodispersions, hydrogels or a Pickering emulsion are also advantageous embodiments.

The consistency of the formulations can range from pasty formulations via flowable formulations to thin-liquid, sprayable products. Accordingly, creams, lotions or sprays can be formulated. For use, the cosmetic preparations according to the invention are applied to the skin in an adequate amount in the manner customary for cosmetics and dermatologicals.

Through the use it is possible to achieve not only uniform skin coloration, it is also possible to evenly color areas of skin that are a different color naturally or as a result of pathological change.

According to the invention, the self-tanning agents used are advantageously, inter alia, glycerol aldehyde, hydroxymethylglyoxal, γ-dialdehyde, erythrulose, 5-hydroxy-1,4-naphthoquinone (juglone), and 2-hydroxy-1,4-naphthoquinone, which occurs in henna leaves.

For the purposes of the invention, 1,3-dihydroxyacetone (DHA), a trivalent sugar occurring in the human body, or the combination of dihydroxyacetone and troxerutin, which is marketed by Merck under the name DHA Rapid®, are very particularly preferred. 6-Aldo-D-fructose and ninhydrin can also be used as self-tanning agents according to the invention. For the purposes of the invention, self-tanning agents are also to be understood as meaning substances which bring about a skin coloration deviating from a brown shade.

In a preferred embodiment of the invention, these preparations comprise two or more self-tanning substances in a concentration of from 0.1 to 10% by weight and particularly preferably from 0.5 to 6% by weight, in each case based on the total weight of the composition.

Preferably, these preparations comprise 1,3-dihydroxyacetone as self-tanning substance. Further preferably, these preparations comprise organic and/or inorganic photoprotective filters. The preparations can also comprise inorganic and/or organic and/or modified inorganic pigments.

Customary and advantageous ingredients further present in the preparations according to the invention are specified above and, for example, in DE 103 21 147 in paragraphs [0024] to [0132], to which reference is made at this point in its entirety.

The invention also provides the use of such preparations for coloring the hair of multicellular organisms, in particular the skin of humans and animals, in particular also for evening up the color of differently pigmented areas of skin.

The invention is illustrated in more detail in the examples below without limiting it thereto.

### EXAMPLES

Preparation of the Amphiphilic Block Copolymers a:

PIBSA=polyisobutene end-functionalized with succinic anhydride.

### Example 1

### Preparation of a Linear Triblock Copolymer ABA:

Reaction of PIBSA₅₅₀ (molar mass Mₙ 550, saponification number, SN=162 mg KOH/g) with Pluriol® E1500 (polyethylene oxide, Mₙ˜1500)

693 g of PIBSA (Mₙ=684; dispersity index PDI=1.7) and 750 g of Pluriol® E1500 were initially introduced into a 4 l three-neck flask with internal thermometer, reflux condenser and nitrogen line. During heating to 80° C., evacuation and aeration with N₂ was carried out 3 times. The reaction mixture was heated to 130° C. and held for 3 h at this temperature. The product was then left to cool to room temperature. IR spectrum (KBr) in cm⁻¹:

OH valence vibration at 3308; C—H valence vibration at 2953, 2893, 2746; C═O valence vibration at 1735; C═C valence vibration at 1639; further vibrations of the PIB structure: 1471, 1390, 1366, 1233; ether vibration of the Pluriol at 1111.

1-H-NMR spectrum (CDCl₃, 500 MHz, TMS, room temperature) in ppm: 4.9-4.7 (C═C of PIBSA); 4.3-4.1 (C(O)—O—CH₂—CH₂—); 3.8-3.5 (O—CH₂—CH₂—O, PEO chain); 3.4 (O—CH₃); 3.1-2.9; 2.8-2.4; 2.3-2.1; 2.1-0.8 (methylene and methine of the PIB chain)

### Example 2

### Preparation of a Linear Triblock Copolymer ABA:

Reaction of PIBSA₅₅₀ (saponification number, SN=162 mg KOH/g) with Pluriol® E6000 (polyethylene oxide, Mₙ˜6000)

346 g of PIBSA (Mₙ=684; PDI=1.7) and 1500 g of Pluriol® E6000 were initially introduced into a 4 l three-neck flask with internal thermometer, reflux condenser and nitrogen line. During heating to 80° C., evacuation and aeration with N₂ were carried out 3 times. The mixture was then heated to 130° C. and held at this temperature for 3 h. The product was then left to cool to room temperature.

IR spectrum (KBr) in cm⁻¹:

OH valence vibration at 3310; C—H valence vibration at 2952, 2893, 2743; C═O valence vibration at 1736; C═C valence vibration at 1639; further vibrations of the PIB structure: 1470, 1389, 1366, 1235; ether vibration of the Pluriol at 1110.

1-H-NMR spectrum (CDCl₃, 500 MHz, TMS, room temperature) in ppm: comparable with example 1, different intensities: 4.9-4.7 (C═C of PIBSA); 4.3-4.1 (C(O)—O—CH₂—CH₂—); 3.8-3.5 (O—CH₂—CH₂—O, PEO chain); 3.4 (O—CH₃); 3.1-2.9; 2.8-2.4; 2.3-2.1; 2.1-0.8 (methylene and methine of the PIB chain)

### Example 3

### Preparation of a Linear Triblock Copolymer ABA:

Reaction of PIBSA₁₀₀₀ (saponification number, SN=97 mg KOH/g) with Pluriol® E4000 (polyethylene oxide, Mₙ˜4000)

578 g of PIBSA (Mₙ=1157; PDI=1.55) and 1000 g of Pluriol® E4000 were initially introduced into a 4 l three-neck flask with internal thermometer, reflux condenser and nitrogen line. During heating to 80° C., evacuation and aeration with N₂ were carried out 3 times. The mixture was then heated to 130° C. and held at this temperature for 3 h. The product was then left to cool to room temperature.

IR spectrum (KBr) in cm⁻¹:

OH valence vibration at 3312; C—H valence vibration at 2957, 2890, 2744; C═O valence vibration at 1730; C═C valence vibration at 1640; further vibrations of the PIB structure: 1470, 1388, 1365, 1232; ether vibration of the Pluriol at 1108.

1-H-NMR spectrum (CDCl₃, 500 MHz, TMS, room temperature) in ppm: comparable with example 1, different intensities: 4.9-4.7 (C═C of PIBSA); 4.3-4.1 (C(O)—O—CH₂—CH₂—); 3.8-3.5 (O—CH₂—CH₂—O, PEO chain); 3.4 (O—CH₃); 3.1-2.9; 2.8-2.4; 2.3-2.1; 2.1-0.8 (methylene and methine of the PIB chain)

### Example 4

### Preparation of a Linear Triblock Copolymer ABA:

Reaction of PIBSA₅₅₀ (saponification number, SN=162 mg KOH/g) with Pluriol® E12000 (polyethylene oxide, Mₙ˜12000)

240 g of PIBSA (Mₙ=684; PDI=1.7) and 2100 g of Pluriol® E12000 were initially introduced into a 4 l three-neck flask with internal thermometer, reflux condenser and nitrogen line. During heating to 80° C., evacuation and aeration with N₂ were carried out 3 times. The mixture was then heated to 130° C. and held at this temperature for 3 h. The product was then left to cool to room temperature.

IR spectrum (KBr) in cm⁻¹:

OH valence vibration at 3309; C—H valence vibration at 2950, 2892, 2744; C═O valence vibration at 1738; C═C valence vibration at 1640; further vibrations of the PIB structure: 1471, 1389, 1367, 1234; ether vibration of the Pluriol at 1111.

1-H-NMR spectrum (CDCl₃, 500 MHz, TMS, room temperature) in ppm: comparable with example 1, different intensities: 4.9-4.7 (C═C of PIBSA); 4.3-4.1 (C(O)—O—CH₂—CH₂—); 3.8-3.5 (O—CH₂—CH₂—O, PEO chain); 3.4 (O—CH₃); 3.1-2.9; 2.8-2.4; 2.3-2.1; 2.1-0.8 (methylene and methine of the PIB chain)

### Example 5

### Preparation of a Diblock Copolymer AB:

Reaction of PIBSA₅₅₀ (saponification number, SN=162 mg KOH/g) with Pluriol® A350E (polyethylene oxide monomethyl ether, Mₙ˜350)

1042 g of PIBSA (Mₙ=684; PDI=1.7) were initially introduced into a 2 l three-neck flask with internal thermometer, dropping funnel and nitrogen line. During heating to 80° C., evacuation and aeration with N₂ were carried out 3 times. Following the addition of 525 g of Pluriol via the dropping funnel, the mixture was heated to 140° C. and held at this temperature for 3 h. The product was then left to cool to room temperature.

IR spectrum (KBr) in cm⁻¹:

OH valence vibration at 3308; C—H valence vibration at 2951, 2893, 2745; C═O valence vibration at 1736; C═C valence vibration at 1639; further vibrations of the PIB structure: 1471, 1389, 1366, 1233; ether vibration of the Pluriol at 1112.

1-H-NMR spectrum (CDCl₃, 500 MHz, TMS, room temperature) in ppm: 4.9-4.7 (C═C of PIBSA); 4.3-4.1 (C(O)—O—CH₂—CH₂—); 3.8-3.5 (O—CH₂—CH₂—O, PEG chain); 3.4 (O—CH₃); 3.1-2.9; 2.8-2.4; 2.3-2.1; 2.1-0.8 (methylene and methine of the PIB chain)

GPC (styrene standard, THF):

Mₙ=1182; M_{w}=1479; M_{z}=1702; polydispersity PDI=1.25

### Example 6

### Preparation of a Diblock Copolymer AB:

Reaction of PIBSA₅₅₀ (saponification number, SN=162 mg KOH/g) with Pluriol® A500E (polyethylene oxide monomethyl ether, Mₙ˜500)

970 g of PIBSA (Mₙ=684; PDI=1.7) were initially introduced into a 2 l three-neck flask with internal thermometer, dropping funnel and nitrogen line. During heating to 80° C., evacuation and aeration with N₂ were carried out 3 times. Following the addition of 700 g of Pluriol via the dropping funnel, the mixture was heated to 140° C. and held at this temperature for 3 h. The product was then left to cool to room temperature.

IR spectrum (KBr) in cm⁻¹:

OH valence vibration at 3310; C—H valence vibration at 2952, 2893, 2743; C═O valence vibration at 1734; C═C valence vibration at 1639; further vibrations of the PIB structure: 1470, 1389, 1366, 1235; ether vibration of the Pluriol at 1111.

1-H-NMR spectrum (CDCl₃, 500 MHz, TMS, room temperature) in ppm: comparable with example 1, different intensities: 4.9-4.7 (C═C of PIBSA); 4.3-4.1 (C(O)—O—CH₂—CH₂—); 3.8-3.5 (O—CH₂—CH₂—O, PEG chain); 3.4 (O—CH₃); 3.1-2.9; 2.8-2.4; 2.3-2.1; 2.1-0.8 (methylene and methine of the PIB chain) GPC (styrene standard, THF):

Mₙ=1315; M_{w}=1611; M_{z}=1838; PDI=1.22

### Example 7

### Preparation of a Diblock Copolymer AB:

Reaction of PIBSA₁₀₀₀ (saponification number, SN=95 mg KOH/g) with Pluriol®A350E (polyethylene oxide monomethyl ether, Mₙ˜350)

1300 g of PIBSA (Mₙ=1320; PDI=1.5) were initially introduced into a 2 l three-neck flask with internal thermometer, dropping funnel and nitrogen line. During heating to 80° C., evacuation and aeration with N₂ were carried out 3 times. Following the addition of 385 g of Pluriol via the dropping funnel, the mixture was heated to 140° C. and held at this temperature for 3 h. The product was then left to cool to room temperature.

IR spectrum (KBr) in cm⁻¹:

OH valence vibration at 3306; C—H valence vibration at 2954, 2894, 2744; C═O valence vibration at 1732; C═C valence vibration at 1640; further vibrations of the PIB structure: 1471, 1390, 1366, 1234; ether vibration of the Pluriol at 1108.

1-H-NMR spectrum (CDCl₃, 500 MHz, TMS, room temperature) in ppm: comparable with example 1, different intensities: 4.9-4.7 (C═C of PIBSA); 4.3-4.1 (C(O)—O—CH₂—CH₂—); 3.8-3.5 (O—CH₂—CH₂—O, PEG chain); 3.4 (O—CH₃); 3.1-2.9; 2.8-2.4; 2.3-2.1; 2.1-0.8 (methylene and methine of the PIB chain)

GPC (styrene standard, THF):

Mₙ=1699; M_{w}=2213; M_{z}=2745; PDI=1.30

### Example 8

### Preparation of a Diblock Copolymer AB:

Reaction of PIBSA₁₀₀₀ (saponification number, SN=95 mg KOH/g) with Pluriol® A500E (polyethylene oxide monomethyl ether, Mₙ˜500)

1180 g of PIBSA (Mₙ=1320; PDI=1.5) were initially introduced into a 2 l three-neck flask with internal thermometer, dropping funnel and nitrogen line. During heating to 80° C., evacuation and aeration with N₂ were carried out 3 times. Following the addition of 500 g of Pluriol via the dropping funnel, the mixture was heated to 140° C. and kept at this temperature for 3 h. The product was then left to cool to room temperature.

IR spectrum (KBr) in cm⁻¹:

OH valence vibration at 3306; C—H valence vibration at 2951, 2893, 2745; C═O valence vibration at 1736; C═C valence vibration at 1639; further vibrations of the PIB structure: 1471, 1389, 1366, 1233; ether vibration of the Pluriol at 1112.

1-H-NMR spectrum (CDCl₃, 500 MHz, TMS, room temperature) in ppm: comparable with example 1, different intensities: 4.9-4.7 (C═C of PIBSA); 4.3-4.1 (C(O)—O—CH₂—CH₂—); 3.8-3.5 (O—CH₂—CH₂—O, PEG chain); 3.4 (O—CH₃); 3.1-2.9; 2.8-2.4; 2.3-2.1; 2.1-0.8 (methylene and methine of the PIB chain)

GPC (styrene standard, THF):

Mₙ=1784; M_{w}=2309; M_{z}=2896; PDI=1.29

### Example 9

### Preparation of a Diblock Copolymer AB:

Reaction of PIBSA₅₅₀ (saponification number, SN=156 mg KOH/g) with Lutensol® AO 30 (polyethylene oxide monoalkyl ether RO(CH₂CH₂O)ₓH, R═C₁₃C₁₅-oxo alcohol, x=30)

180 g of PIBSA₅₅₀ (Mₙ=719; PDI=1.7) and 383 g of Lutensol® AO 30 (Mₙ˜1530) were initially introduced into a 1 l flask with internal thermometer and nitrogen line. During heating to 80° C., evacuation and aeration with N₂ were carried out 3 times. The mixture was then heated to 130° C. and held at this temperature for 3 h. The product was then left to cool to room temperature.

IR spectrum (KBr) in cm⁻¹:

OH valence vibration at 3312; C—H valence vibration at 2950, 2888, 2746; C═O valence vibration at 1733; C═C valence vibration at 1640; further vibrations of the PIB structure: 1470, 1388, 1365, 1232; ether vibration of the Pluriol at 1109.

### Example 10

### Preparation of the Diblock Copolymer AB:

Reaction of PIBSA₁₀₀₀ (saponification number, SN=87.5 mg KOH/g) with Lutensol® AT 80 (polyethylene oxide monoalkyl ether RO(CH₂CH₂O)ₓH, R═C₁₆C₁₈ fatty alcohol, x=80)

128 g of PIBSA₁₀₀₀ (Mₙ=1282; PDI=1.5) and 378 g of Lutensol® AT 80 (Mₙ˜3780) were initially introduced into a 1 l flask with internal thermometer and nitrogen line. While heating to 80° C., evacuation and aeration with N₂ were carried out 3 times. The mixture was then heated to 130° C. and held at this temperature for 3 h. The product was then left to cool to room temperature.

IR spectrum (KBr) in cm⁻¹:

OH valence vibration at 3320; C—H valence vibration at 2954, 2891, 2747; C═O valence vibration at 1738; C═C valence vibration at 1642; further vibrations of the PIB structure: 1471, 1390, 1368, 1237; ether vibration of Pluriol at 1114.

### Example 11

### Preparation of a Linear Triblock Copolymer ABA:

Reaction of PIBSA₁₀₀₀ (saponification number, SN=87.5 mg KOH/g) with Pluriol® P 900 (polypropylene oxide, Mₙ˜900)

385 g of PIBSA₁₀₀₀ (Mₙ=1282; dispersity index PDI=1.5) and 136 g of Pluriol® P 900 were initially introduced into a 1 l flask with internal thermometer, reflux condenser and nitrogen line. During heating to 80° C., evacuation and aeration with N₂ were carried out 3 times. The reaction mixture was then heated to 130° C. and held at this temperature for 3 h. The product was then left to cool to room temperature.

IR spectrum (KBr) in cm⁻¹:

C—H valence vibration at 2941, 2882; C═O valence vibration at 1732; C═C valence vibration at 1644; further vibrations of the PIB structure: 1472, 1393, 1364, 1236; ether vibration of Pluriol at 1094.

### Example 12

### Preparation of a Linear Triblock Copolymer ABA:

Reaction of PIBSA₁₀₀₀ (saponification number, SN=87.5 mg KOH/g) with Pluronic® PE 6400 (block copolymer of polypropylene oxide (PPO) and polyethylene oxide (PEO) with PEO-PPO-PEO structure, Mₙ˜2900, with 60% by weight of PPO and 40% by weight of PEO)

256 g of PIBSA₁₀₀₀ (Mₙ=1282; dispersity index PDI=1.5) and 290 g of Pluronic® PE 6400 were initially introduced into a 1 l flask with internal thermometer and nitrogen line. During heating to 80° C., evacuation and aeration with N₂ were carried out 3 times. The reaction mixture was then heated to 130° C. and held at this temperature for 3 h. The product was then left to cool to room temperature.

IR spectrum (KBr) in cm⁻¹:

C—H valence vibration at 2948, 2891; C═O valence vibration at 1730; C═C valence vibration at 1646; further vibrations of the PIB structure: 1471, 1395, 1364, 1237; ether vibration of Pluronics at 1101.

### Example 13

### Preparation of a Branched Block Copolymer A₃B₃:

Reaction of PIBSA₅₅₀ (saponification number, SN=156 mg KOH/g) with an ethoxylated glycerol (OH number=540 mg KOH/g, Mₙ˜310)

503 g of PIBSA₅₅₀ (Mₙ=719; dispersity index PDI=1.7) and 73 g of ethoxylated glycerol were initially introduced into a 2 l flask with internal thermometer and nitrogen line. During heating to 80° C., evacuation and aeration with N₂ were carried out 3 times. The reaction mixture was then heated to 130° C. and held at this temperature for 3 h. The product was then cooled to room temperature.

IR spectrum (KBr) in cm⁻¹:

OH valence vibration at 3305; C—H valence vibration at 2951, 2890; C═O valence vibration at 1738; C═C valence vibration at 1640; further vibrations of the PIB structure: 1472, 1389, 1366, 1232; ether vibration at 1115.

### Example 14

### Preparation of a Linear Block Copolymer AB:

Reaction of PIBSA₁₀₀₀ (saponification number, SN=87.5 mg KOH/g) with a polyethyleneimine (Mₙ˜450)

1290 g of PIBSA₁₀₀₀ (Mₙ=1282; PDI=1.5) in 200 ml of toluene were initially introduced into a 3 l flask with internal thermometer, dropping funnel, reflux condenser and nitrogen line and rendered inert with nitrogen. 450 g of polyethyleneimine were added dropwise via the dropping funnel. After the addition had taken place, the mixture was heated at 120° C. for 2 h. The toluene was then distilled off.

IR spectrum (KBr) in cm⁻¹:

NH valence vibration at 3292; C—H valence vibration at 2951, 2896, 2838; C═O valence vibration at 1701; C═C valence vibration at 1654; further vibrations of the PIB structure: 1471, 1389, 1366, 1231.

### Example 15

### Preparation of a Linear Block Copolymer AB:

Hydroboration of a polyisobutene (Mₙ=550) and subsequent propoxylation by means of DMC catalysis

20 g of NaBH₄ and 100 g of BF₃*OEt₂ in 150 ml of THF were reacted at 0° C. in a 4 l flask with internal thermometer, dropping funnel, reflux condenser and nitrogen line. 550 g of PIB in 300 ml of THF were then added dropwise. When the addition was complete, the mixture was warmed to room temperature. Then, 50 ml of water, 500 ml of 10% NaOH and 500 ml of 30% H₂O₂ were added dropwise in succession. After 3 h at room temperature, the product mixture was worked up by means of phase separation. The solvent of the organic phase was distilled off. The polyisobutene alcohol obtained was used for the subsequent reaction.

245 g of polyisobutene alcohol were initially introduced with 500 ppm of DMC catalyst at 120° C. into a 1 l autoclave. 200 g of propylene oxide were metered in at a metering rate of 150 g/h. After cooling and decompression of the reaction vessel, the catalyst was filtered off.

OH number: 27 mg KOH/g

1-H-NMR spectrum (CDCl₃, 500 MHz, TMS, room temperature) in ppm:

3.2-3.7 (O—CH(CH₃)—CH₂) of the PPO chain); 0.8-2.0 (methylene and methine of the PIB chain)

GPC (styrene standard, THF):

Mₙ=1766; M_{w}=2461; M_{z}=11800; PDI=1.39

### Example 16

### Preparation of a Linear Block Copolymer AB:

Hydrogenation of a polyisobutenephenol (Mₙ=1000) and subsequent ethoxylation by means of KOH catalysis

1100 g of a 4-polyisobutenephenol which have been prepared from a polyisobutene (Mₙ=1000) were dissolved in 500 ml of heptane. The solution was treated with 500 mg of NaH and transferred to a 3 l autoclave. After adding 50 g of Raney nickel catalyst, a hydrogen pressure of 150 bar was established. Subsequently, stirring was carried out for 2 h at 100° C. and for 1 h at 150° C. Following cooling and decompression, the Raney nickel catalyst was filtered off and the solvent was distilled off. The polyisobutenecyclohexanol obtained was used for the subsequent reaction.

220 g of polyisobutenecyclohexanol were initially introduced with 2000 ppm of KOH at 120° C. in a 1 l autoclave. At a metering rate of 100 g/h, 440 g of ethylene oxide were metered in. After cooling and decompression of the reaction vessel, the catalyst was filtered off.

OH number: 17 mg KOH/g

1-H-NMR spectrum (CDCl₃, 500 MHz, TMS, room temperature) in ppm:

3.4-3.8 (O—CH₂—CH₂) of the PEO chain); 0.8-2.0 (methylene and methine of the PIB chain and of the cyclohexane ring)

GPC (styrene standard, THF):

Mₙ=3189; M_{w}=4632; PDI=1.45

### Example 17

### Preparation of a Branched Block Copolymer AB₂:

Reaction of a polyisobutenephosphonoyl dichloride (PIB radical with Mₙ=1000) with Pluriol® A350E (polyethylene oxide monomethyl ether, Mₙ˜350)

100 g of polyisobutene (Mₙ=1000; PDI=1.65) and 100 ml of hexane were initially introduced at room temperature into a 500 ml flask and heated to 50° C. At 50° C., 42 g of PCl₅ were added to the solution and the mixture was afterstirred for 2 h. 21 g of acetic anhydride were then added dropwise at 50° C. After 30 min, the volatile constituents were distilled off at 100° C. and 5 mbar. The resulting product (polyisobutenephosphonoyl dichloride) was isolated.

140 g of Pluriol® A350E were initially introduced with 32 g of dry pyridine in 150 ml of toluene at 5° C. into a 1 l flask. 130 g of the polyisobutenephosphonoyl dichloride in 100 ml of toluene were added dropwise. The mixture was left to warm to room temperature and stirred overnight at 40° C. The precipitated-out pyridinium chloride was filtered off. The solvent was distilled off on a rotary evaporator at 80° C. and 2 mbar.

IR spectrum (KBr) in cm⁻¹:

C—H valence vibration at 2951, 2892; further vibrations of the PIB structure: 1471, 1389, 1368, 1234; P═O at 1200; P—O-alkyl at 1135; ether vibration of the Pluriol at 1112.

### Example 18

### Preparation of a Linear Block Copolymer BAB:

Hydroboration of a polyisobutene (Mₙ=2000) with two reactive chain ends to the polyisobutenediol and subsequent propoxylation by means of DMC catalysis

A solution of 364 ml of isobutene, 3.1 g of phenyltriethoxysilane and 31.4 g of 1,4-bis(o-chloroisopropyl)benzene in 400 ml of hexane and 400 ml of CH₂Cl₂ was admixed at −78° C. with 4.9 g of TiCl₄ and stirred for 2 h at −50° C. The polymerization was then interrupted with 10 ml of isopropanol, the reaction solution was warmed to room temperature, washing with water was carried out 3 times and the solvent was distilled off. Drying was then carried out at 150° C. at 2 mbar. This gives a polyisobutene with two reactive chain ends (PDI=1.33; Mₙ=1924).

2 g of NaBH₄ and 10 g of BF₃*OEt₂ in 50 ml of THF were reacted at 0° C. in a 1 l flask with internal thermometer, dropping funnel, reflux condenser and nitrogen line. 70 g of bifunctional PIB (from the above batch) in 200 ml of THF were then added dropwise. When the addition was complete, the mixture was warmed to room temperature. 50 ml of water, 150 ml of 10% NaOH and 70 ml of 30% H₂O₂ were then added dropwise in succession. After 3 h at room temperature, the product mixture was worked up by means of phase separation. The solvent of the organic phase was distilled off. The resulting polyisobutenediol was used for the subsequent reaction.

70 g of polyisobutenediol were initially introduced with 500 ppm of DMC catalyst at 120° C. into a 1 l autoclave. At a metering rate of 150 g/h, 80 g of propylene oxide were metered in. After cooling and decompression of the reaction vessel, the catalyst was filtered off.

OH number: 25 mg KOH/g

1-H-NMR spectrum (CD₂Cl₂, 500 MHz, TMS, room temperature) in ppm: 7.27 (aromatic protons of the initiator); 3.2-3.7 (O—CH(CH₃)—CH₂) of the PPO chain); 0.8-2.0 (methylene and methine of the PIB chain)

GPC (styrene standard, THF):

Mₙ=4089; M_{w}=5126; PDI=1.25

Application Examples

Examples of Preparations

The quantitative data are in % by weight unless noted otherwise. The preparations specified below are preferably provided in the respective customary devices known to the person skilled in the art; for example in bottles, tubes, squeezable bottles, cans, spray cans, pots, in impregnated wipes, spray bottles, pump spray bottles, flacons etc . . . Examples of O/W skin cream formulations

| Phase | Ingredient | INCI | O/W 1 | O/W 2 | O/W 3 | O/W 4 |
|---|---|---|---|---|---|---|
| A | Abil ® Care 85 | Bis-PEG/PPG-16/16 PEG/PPG- | 5.00 | 5.00 | 6.50 | 4.50 |
| | | 16/16 Dimethicone, | | | | |
| | | Caprylic/Capric Triglyceride | | | | |
| | Cremophor ® CO | PEG-40 Hydrogenated Castor | 3.00 | 2.00 | 4.00 | 3.50 |
| | 40 | Oil | | | | |
| | Cremophor ® WO 7 | PEG-7 Hydrogenated Castor Oil | 0.30 | 0.20 | 0.25 | 0.40 |
| | Uvinul ® A PLUS | Diethylamino Hydroxybenzoyl | 0.90 | 3.00 | 6.50 | 0 |
| | | Hexyl Benzoate | | | | |
| | Uvinul ® MC 80 | Ethylhexyl Methoxycinnamate | 5.00 | 3.00 | 0 | 1.00 |
| | Polymer Example 1 | | 5.00 | 8.00 | 3.50 | 10.00 |
| | Witconol ® APM | PPG-3 Myristyl Ether | 10.00 | 5.00 | 7.00 | 8.00 |
| | Uvinul ® T 150 | Ethylhexyl Triazone | 2.00 | 1.00 | 4.00 | 1.00 |
| | Dow | Cyclopentasiloxane, | 1.00 | 2.00 | 1.50 | 0.50 |
| | Corning ® 345 fluid | Cyclohexasiloxane | | | | |
| | Uvinul ® N 539 | Octocrylene | 5.00 | 0 | 7.00 | 2.00 |
| B | Z-Cote ® HP 1 | Zinc Oxide, | 5.00 | 7.00 | 8.90 | 10.00 |
| | | Triethoxycaprylylsilane | | | | |
| C | 1,2 Propylene | Propylene Glycol | 5.00 | 7.50 | 4.00 | 8.00 |
| | Glycol Care | | | | | |
| | D-Panthenol 50 P | Panthenol, Propylene Glycol | 2.0 | 1.50 | 0.50 | 0.75 |
| | Edeta BD | Disodium EDTA | 0.10 | 0.20 | 0.40 | 0.25 |
| | Keltrol | Xanthan Gum | 0.2 | 0.40 | 0.25 | 0 |
| | Simulgel ® 600 | Acrylamide/Sodium | 1.5 | 1.30 | 1.80 | 2.00 |
| | | Acryloyldimethyltaurate | | | | |
| | | Copolymer, Isohexadecane, | | | | |
| | | Polysorbate 80 | | | | |
| | Water dem. | Aqua dem. | ad | ad | ad | ad |
| | | | 100 | 100 | 100 | 100 |
| C | Preservative | | 0.25 | 0.25 | 0.25 | 0.25 |

Preparation:

Heat phase A and C to 80° C. Homogenize phase B into phase. Prehomogenize phase C and stir into phase A+B. Briefly afterhomogenize and cool to 40° C. and incorporate phase D. The analogous formulation is prepared analogously also with the copolymers from the preparation examples 2-18.

Examples of hydrodispersion formulations (quantitative data in % by weight)

| Phase | Ingredient | INCI | HD 5 | HD 6 | HD 7 | HD 8 |
|---|---|---|---|---|---|---|
| A | Pemulen ® TR-1 | Acrylates/C10-30 Alkyl Acrylate | 0.30 | 0.30 | 0.30 | 0.30 |
| | | Crosspolymer | | | | |
| B | Luvigel ® EM | Caprylic/Capric Triglyceride, | 1.00 | 1.25 | 2.00 | 2.50 |
| | | Sodium Acrylates Copolymer | | | | |
| | Fitoderm ® | Squalane | 5.00 | 0 | 3.50 | 7.00 |
| | Polymer Example 1 | | 2.00 | 3.50 | 1.00 | 4.50 |
| | Crodamol ® PTC | Pentaerythrityl | 5.00 | 4.00 | 5.00 | 0 |
| | | Tetracaprylate/Tetracaprate | | | | |
| | Jojoba Oil | Simmondsia Chinensis (Jojoba) | 5.00 | 4.00 | 3.50 | 2.00 |
| | | Seed Oil | | | | |
| | D,L-Alpha- | Tocopherol | 0.10 | 0 | 0.20 | 0.25 |
| | Tocopherol | | | | | |
| | Vitamin-E | Tocopheryl Acetate | 0.50 | 0 | 0.50 | 2.00 |
| | Acetate | | | | | |
| | Cremophor CO | PEG-40 Hydrogenated Castor Oil | 1.00 | 1.00 | 1.00 | 1.00 |
| | 40 | | | | | |
| | RetiStar ® | Caprylic/Capric Triglyceride, | 1.00 | 0.70 | 1.00 | 0.50 |
| | | Sodium Ascorbate, Tocopherol, | | | | |
| | | Retinol | | | | |
| | Preservative | | 0.50 | 0.50 | 0.50 | 0.50 |
| C | 1,2 Propylene | Propylene Glycol | 5.00 | 4.00 | 7.00 | 8.00 |
| | Glycol Care | | | | | |
| | Edeta BD | Disodium EDTA | 0.10 | 0.10 | 0.10 | 0.10 |
| | Water dem. | Aqua dem. | ad | ad | ad | ad |
| | | | 100 | 100 | 100 | 100 |
| D | Triethanolamine | Triethanolamine | 0.40 | 0.40 | 0.40 | 0.40 |

Preparation:

Disperse phase A into phase B. Stir phase C into phase A+B and homogenize. Neutralize with phase D and briefly afterhomogenize. The analogous formulation is prepared analogously also with the copolymers from the preparation examples 2-18.

Examples of Cream Gels

| | | | | Gel | Gel | Gel | Gel | Gel |
|---|---|---|---|---|---|---|---|---|
| Phase | Ingredient | INCI | Gel 9 | 10 | 11 | 12 | 13 | 14 |
| A | Menthol | Menthol | 0.30 | 0.30 | 0.30 | 0.30 | 0.30 | 0.30 |
| | Perfume oil | Fragrance | 0.5 | 0.5 | 0.5 | 0.5 | 0.5 | 0.5 |
| | “Ocean | | | | | | | |
| | Fresh” | | | | | | | |
| | Luvigel ® EM | Caprylic/Capric | 2.50 | 2.50 | 3.50 | 2.00 | 2.50 | 2.50 |
| | | Triglyceride, | | | | | | |
| | | Sodium Acrylate | | | | | | |
| | | Copolymer | | | | | | |
| | Polymer | | 2.00 | 3.50 | 1.00 | 4.50 | 0.50 | 2.50 |
| | Example 2 | | | | | | | |
| | Cremophor ® | PEG-40 | 3.00 | 3.00 | 3.00 | 3.00 | 3.00 | 3.00 |
| | CO 40 | Hydrogenated | | | | | | |
| | | Castor Oil | | | | | | |
| B | Water dem. | Aqua dem. | ad | ad | ad | ad | ad | ad |
| | | | 100 | 100 | 100 | 100 | 100 | 100 |
| C | Ethanol 96% | Alcohol | 15.00 | 15.00 | 15.00 | 15.00 | 15.00 | 15.00 |
| | Glycerol 87% | Glycerol | 3.00 | 3.00 | 3.00 | 3.00 | 3.00 | 3.00 |

Preparation:

Homogeneously mix phase A and stir phase B into phase A and then slowly stir in phase C. The analogous formulation is prepared analogously also with preparation examples 1 and 3-18.

Hydrodispersion Examples

| | | | HD | HD | HD | HD |
|---|---|---|---|---|---|---|
| Phase | Ingredient | INCI | 15 | 16 | 17 | 18 |
| A | D-Panthenol 50 P | Panthenol, Propylene Glycol | 5.00 | 4.00 | 3.50 | 2.50 |
| | Urea | Urea | 1.00 | 3.00 | 3.50 | 5.00 |
| | Glycerin 87% | Glycerin | 2.00 | 4.00 | 6.00 | 2.50 |
| | Aristoflex ® AVC | Ammonium | 1.20 | 1.2 | 1.30 | 1.30 |
| | | Acryloyldimethyltaurate/VP | | | | |
| | | Copolymer | | | | |
| | Polymer | | 2.50 | 6.50 | 3.00 | 4.50 |
| | Example 3 | | | | | |
| | Water dem. | Aqua dem. | ad | ad | ad | ad |
| | | | 100 | 100 | 100 | 100 |
| B | Cremophor ® CO | PEG-40 Hydrogenated Castor Oil | 1.00 | 0.50 | 3.00 | 2.70 |
| | 40 | | | | | |
| | Perfume oil | Fragrance | 0.10 | 0.10 | 0.10 | 0.15 |
| | Miglyol ® 812 | Caprylic/Capric Triglyceride | 1.00 | 2.00 | 4.50 | 2.00 |
| | Uvinul ® A Plus B | Ethylhexyl Methoxycinnamate, | 10.00 | 7.00 | 5.50 | 3.40 |
| | | Diethylamino Hydroxybenzoyl | | | | |
| | | Hexyl Benzoate | | | | |
| | Preservative | | 0.1 | 0.1 | 0.1 | 0.1 |

Preparation:

Allow phase A to swell and stir until homogeneous. Mix phase B and stir into phase A. Briefly homogenize. The analogous formulation is prepared analogously also with the copolymers from preparation examples 1 and 2 and 4-18.

Hydrodispersion Sun Care Examples

| | | | HD | HD | HD | HD |
|---|---|---|---|---|---|---|
| Phase | Ingredient | INCI | 19 | 20 | 21 | 22 |
| A | Uvinul ® MC 80 | Ethylhexyl | 7.50 | 5.00 | 3.00 | 7.00 |
| | | Methoxycinnamate | | | | |
| | Uvinul ® A Plus | Diethylamino Hydroxybenzoyl | 2.00 | 5.00 | 2.40 | 5.00 |
| | | Hexyl Benzoate | | | | |
| | Uvinul ® N 539 T | Octocrylene | 3.00 | 10.00 | 0 | 3.00 |
| | Cremophor ® CO | PEG-40 Hydrogenated | 1.00 | 1.50 | 1.50 | 1.10 |
| | 40 | Castor Oil | | | | |
| | Polymer | | 0.50 | 10.50 | 5.00 | 2.50 |
| | Example 1 | | | | | |
| | Miglyol ® 812 | Caprylic/Capric Triglyceride | 10.00 | 4.00 | 7.50 | 2.50 |
| | DC 345 ® fluid | Cyclopentasiloxane, | 1.50 | 0 | 4.00 | 1.00 |
| | | Cyclohexasiloxane | | | | |
| B | Luvigel ® EM | Caprylic/Capric Triglyceride, | 2.00 | 1.70 | 3.50 | 2.50 |
| | | Sodium Acrylate Copolymer | | | | |
| C | Water dem. | Aqua dem. | ad | ad | ad | ad |
| | | | 100 | 100 | 100 | 100 |
| D | 1,2 Propylene | Propylene Glycol | 5.00 | 3.00 | 10.00 | 7.50 |
| | Glycol Care | | | | | |
| | Cremophor ® A 25 | Ceteareth-25 | 0.50 | 1.50 | 1.00 | 0 |
| | Ethanol 96% | Alcohol | 20.00 | 10.00 | 5.00 | 15.00 |
| | Perfume oil | Fragrance | 0.10 | 0.10 | 0.10 | 0.10 |

Preparation:

Mix phase A until homogeneous and stir in phase B. Stir phase C into phase A+B and homogenize. Slowly stir in phase D and briefly afterhomogenize. The analogous formulation is prepared analogously also with the copolymers from preparation examples 2-18.

### Application Example 23

### O/W Cream for Skin Moisturization

| Additive | % by wt. |
|---|---|
| Glycerol monostearate | 2.0 |
| Cetyl alcohol | 3.0 |
| Polymer Example 2 | 5.0 |
| Vaseline | 3.0 |
| Caprylic/capric triglyceride | 4.0 |
| Octyldodecanol | 2.0 |
| Hydrogenated coconut fat | 2.0 |
| Cetyl phosphate | 0.4 |
| Vinylpyrrolidone/acrylic acid/stearyl methacrylate polymer | 3.0 |
| 60/5/35% by wt. (K value 41; 1% in isopropanol) | |
| Glycerol | 3.0 |
| Sodium hydroxide | q.s. |
| Perfume oil | q.s. |
| Preservative | q.s. |
| Water | ad 100 |

Application example 24 to 40. The analogous formulation is prepared analogously also with the copolymers from preparation examples 1 and 3-18.

### Application Example 41

### Liquid Soap

| Additive | % by wt. |
|---|---|
| Coconut fatty acid, potassium salt | 15 |
| Potassium oleate | 3 |
| Polymer Example 1 | 5 |
| Vinylpyrrolidone/stearyl methacrylate polymer 70/30% by wt. | 2 |
| (K value 47; 1% in isopropanol) | |
| Glycerol stearate | 1 |
| Ethylene glycol distearate | 2 |
| Specific additives, complexing agents, fragrances, water | ad 100 |

Application example 42 to application example 58: Application example 41 is repeated except that instead of the polymer from example 1 the copolymers from preparation examples 2-18 are used.

### Application Examples 59-63 PIT—Emulsions:

| | App. | App. | App. | App. | App. |
|---|---|---|---|---|---|
| Additive | Ex. 59 | Ex. 60 | Ex. 61 | Ex. 62 | Ex. 63 |
| Glycerol monostearate self-emulsifying | 0.50 | | 3.00 | 2.00 | 4.00 |
| Polyoxyethylene(12) cetylstearyl ether | | 5.00 | | 1.00 | 1.50 |
| Polyoxyethylene(20) cetylstearyl ether | | | | 2.00 | |
| Polyoxyethylene(30) cetylstearyl ether | 5.00 | | 1.00 | | |
| Stearyl alcohol | | | 3.00 | | 0.50 |
| Cetyl alcohol | 2.50 | 1.00 | | 1.50 | |
| 2-Ethylhexyl methoxycinnamate | | | | 5.00 | 8.00 |
| 2,4-Bis(4-(2-ethylhexyloxy)-2-hydroxyl)- | | 1.50 | | 2.00 | 2.50 |
| phenyl)-6-(4-methoxyphenyl)(1,3,5)- | | | | | |
| triazine | | | | | |
| Butyldimethoxydibenzoylmethane | | | 2.00 | | |
| Diethylamino Hydroxybenzoyl Hexyl | 0.5 | | 2.00 | 3.0 | 0.4 |
| Benzoate | | | | | |
| Diethylhexylbutamidotriazone | 1.00 | 2.00 | | 2.00 | |
| Ethylhexyltriazone | 4.00 | | 3.00 | 4.00 | |
| 4-Methylbenzylidenecamphor | | 4.00 | | | 2.00 |
| Octocrylene | | 4.00 | | | 2.50 |
| Phenylene-1,4-bis(monosodium, | | | 0.50 | | 1.50 |
| 2-benzimidazyl-5,7)-disulfonic acid | | | | | |
| Phenylbenzimidazolesulfonic acid | 0.50 | | | 3.00 | |
| C₁₂₋₁₅-Alkyl benzoate | | 2.50 | | | 5.00 |
| Titanium dioxide | 0.50 | 1.00 | | 3.00 | 2.00 |
| Zinc oxide | 2.00 | | 3.00 | 0.50 | 1.00 |
| Dicaprylyl ether | | | 3.50 | | |
| Butylene glycol dicaprylate/dicaprate | 5.00 | | | 6.00 | |
| Dicaprylyl carbonate | | | 6.00 | | 2.00 |
| Dimethicone polydimethylsiloxane | | 0.50 | 1.00 | | |
| Phenylmethylpolysiloxane | 2.00 | | | 0.50 | 0.50 |
| Shea butter (Sheabutter) | | 2.00 | | | 0.50 |
| PVP Hexadecene copolymer | 0.50 | | | 0.50 | 1.00 |
| Glycerol | 3.00 | 7.50 | 5.00 | 7.50 | 2.50 |
| Tocopherol acetate | 0.50 | | 0.25 | | 1.00 |
| Polymer Example 1 | 0.2 | 1.1 | 0.3 | 3.0 | 0.5 |
| Alpha-Glucosylrutin | 0.10 | | 0.20 | | |
| Preservative | q.s. | q.s. | q.s. | q.s. | q.s. |
| Ethanol | 3.00 | 2.00 | 1.50 | | 1.00 |
| Perfume | q.s. | q.s. | q.s. | q.s. | q.s. |
| Water | ad 100 | ad 100 | ad 100 | ad 100 | ad 100 |

The analogous formulation is prepared analogously also with the copolymers from preparation examples 2-18.

### Application Examples 64-68 PIT—emulsions

| | App. Ex. | App. Ex. | App. Ex. | App. Ex. | App. |
|---|---|---|---|---|---|
| Additives | 64 | 65 | 66 | 67 | Ex. 68 |
| Glycerol monostearate self-emulsifying | 0.50 | | 3.00 | 2.00 | 4.00 |
| Polyoxyethylene(12) cetylstearyl ether | | 5.00 | | 1.00 | 1.50 |
| Polyoxyethylene(20) cetylstearyl ether | | | | 2.00 | |
| Polyoxyethylen(30) cetylstearyl ether | 5.00 | | 1.00 | | |
| Stearyl alcohol | | | 3.00 | | 0.50 |
| Cetyl alcohol | 2.50 | 1.00 | | 1.50 | |
| 2-Ethylhexyl methoxycinnamate | | | | 5.00 | 8.00 |
| 2,4-Bis(4-(2-ethylhexyloxy)-2-hydroxyl)- | | 1.50 | | 2.00 | 2.50 |
| phenyl)-6-(4-methoxyphenyl)(1,3,5)-triazine | | | | | |
| Butyldimethoxydibenzoylmethane | | | 2.00 | | |
| Dimethico diethylbenzalmalonate | | 6.50 | | | |
| Diethylhexylbutamidotriazone | 1.00 | 2.00 | | 2.00 | |
| Ethylhexyltriazone | 4.00 | | 3.00 | 4.00 | |
| Hexyl 2-(4′-(diethylamino)-2′- | 1.50 | 4.00 | 3.50 | 5.00 | 2.00 |
| hydroxybenzoyl)benzoate | | | | | |
| Octocrylene | | 4.00 | | | 2.50 |
| Phenylene-1,4-bis(monosodium) | | | 0.50 | | 1.50 |
| 2-benzimidazyl-5,7-disulfonic acid | | | | | |
| Phenylbenzimidazolesulfonic acid | 0.50 | | | 3.00 | |
| C₁₂₋₁₅-Alkyl benzoate | | 2.50 | | | 5.00 |
| Dicaprylyl ether | | | 3.50 | | |
| Butylene glycol dicaprylate/dicaprate | 5.00 | | | 6.00 | |
| Dicaprylyl carbonate | | | 6.00 | | 2.00 |
| Dimethicone polydimethylsiloxane | | 0.50 | 1.00 | | |
| Phenylmethylpolysiloxane | 2.00 | | | 0.50 | 0.50 |
| Shea butter (Sheabutter) | | 2.00 | | | 0.50 |
| PVP Hexadecene copolymer | 0.50 | | | 0.50 | 1.00 |
| Glycerol | 3.00 | 7.50 | 5.00 | 7.50 | 2.50 |
| Tocopherol acetate | 0.50 | | 0.25 | | 1.00 |
| Polymer Example 1 | 0.10 | 1.00 | 0.20 | 0.50 | 1.50 |
| Diethylhexyl 2,6-naphthalate | | | 2.00 | | |
| Alpha-Glucosylrutin | 0.10 | | 0.20 | | |
| DMDM Hydantoin | | 0.25 | | 0.60 | 0.45 |
| Paraben | 0.15 | | 0.50 | 0.30 | |
| Konkaben LMB ® | 0.20 | | 0.40 | | |
| Trisodium EDTA | | 0.80 | | | 1.00 |
| Phenoxyethanol | 0.30 | | | 0.20 | 0.50 |
| Ethanol | 3.00 | 2.00 | 1.50 | | 1.00 |
| Perfume | q.s. | q.s. | q.s. | q.s. | q.s. |
| Water | ad 100 | ad 100 | ad 100 | ad 100 | ad 100 |

The analogous formulation is prepared analogously also with the copolymers from preparation examples 2-18.

### Application Examples 69-73 O/W Emulsions

| | App. | App. | App. | App. | App. |
|---|---|---|---|---|---|
| Additive | Ex. 69 | Ex. 70 | Ex. 71 | Ex. 72 | Ex. 73 |
| Glyceryl stearate citrate | | | 2.00 | | 2.00 |
| Glyceryl stearate self-emulsifying | 4.00 | 3.00 | | | |
| PEG-40 stearate | 1.00 | | | | |
| Polyglyceryl-3 methylglucose distearate | | | | 3.00 | |
| Sorbitan stearate | | | | | 2.00 |
| Stearic acid | | 1.00 | | | |
| Stearyl alcohol | | | 5.00 | | |
| Cetyl alcohol | 3.00 | 2.00 | | 3.00 | |
| Cetylstearyl alcohol | | | | | 2.00 |
| Caprylic/capric triglyceride | 5.00 | 3.00 | 4.00 | 3.00 | 3.00 |
| Octyldodecanol | | | 2.00 | | 2.00 |
| Dicaprylyl ether | | 4.00 | | 2.00 | 1.00 |
| Paraffinum liquidum | 5.00 | 2.00 | | 3.00 | |
| Titanium dioxide | | | 1.00 | | |
| Octocrylene | | | 3.50 | | |
| Butyldimethoxydibenzoylmethane | | | 0.50 | | |
| Polymer Example 1 | 0.90 | 3.5 | 2.7 | 5.5 | 8.0 |
| Tocopherol | 0.10 | | | | 0.20 |
| Biotin | | | 0.05 | | |
| Ethylenediaminetetraacetic acid trisodium | 0.1 | | 0.10 | 0.1 | |
| Preservative | q.s. | q.s. | q.s. | q.s. | q.s. |
| Polyacrylic acid | 3.00 | 0.1 | | 0.1 | 0.1 |
| Sodium hydroxide solution 45% | q.s | q.s. | q.s. | q.s. | q.s. |
| Glycerol | 5.00 | 3.00 | 4.00 | 3.00 | 3.00 |
| Butylene glycol | | 3.00 | | | |
| Perfume | q.s. | q.s. | q.s. | q.s. | q.s. |
| Water | ad 100 | ad 100 | ad 100 | ad 100 | ad 100 |

The analogous formulation is prepared analogously also with the copolymers from preparation examples 2-18.

### Application Examples 74-78 O/W Emulsions

| | App. | App. | App. | App. | App. |
|---|---|---|---|---|---|
| Additive | Ex. 74 | Ex. 75 | Ex. 76 | Ex. 77 | Ex. 78 |
| Glyceryl stearate citrate | | 2.00 | 2.00 | | |
| Glyceryl stearate self-emulsifying | 5.00 | | | | |
| Stearic acid | | | | 2.50 | 3.50 |
| Stearyl alcohol | 2.00 | | | | |
| Cetyl alcohol | | | | 3.00 | 4.50 |
| Cetylstearyl alcohol | | 3.00 | 1.00 | | 0.50 |
| C₁₂₋₁₅-Alkyl benzoate | | 2.00 | 3.00 | | |
| Caprylic/capric triglyceride | 2.00 | | | | |
| Octyldodecanol | 2.00 | 2.00 | | 4.00 | 6.00 |
| Dicaprylyl ether | | | | | |
| Paraffinum liquidum | | 4.00 | 2.00 | | |
| Cyclic dimethylpolysiloxane | | | | 0.50 | 2.00 |
| Dimethicone polydimethylsiloxane | 2.00 | | | | |
| Titanium dioxide | 2.00 | | | | |
| 4-Methylbenzylidenecamphor | 1.00 | | | | |
| Ethylhexyltriazone | | | | | 2.00 |
| Butyldimethoxydibenzoylmethane | 0.50 | | | | 0.50 |
| Polymer Example 1 | 0.30 | 0.10 | 1.00 | 0.50 | 0.10 |
| Tocopherol | | | | | 0.10 |
| Ethylenediaminetetraacetic acid trisodium | | | 0.20 | | 0.20 |
| Preservative | q.s. | q.s. | q.s. | q.s. | q.s. |
| Xanthan gum | | | 0.20 | | |
| Polyacrylic acid | 0.15 | 0.1 | | 0.05 | 0.05 |
| Sodium hydroxide solution 45% | q.s. | q.s. | q.s. | q.s. | q.s. |
| Glycerol | 3.00 | | 3.00 | 5.00 | 3.00 |
| Butylene glycol | | 3.00 | | | |
| Ethanol | | 3.00 | | 3.00 | |
| Perfume | q.s. | q.s. | q.s. | q.s. | q.s. |
| Water | ad 100 | ad 100 | ad 100 | ad 100 | ad 100 |

The analogous formulation is prepared analogously also with the copolymers from preparation examples 2-18.

### Application Examples 79-83 O/W Emulsions

| | App. | App. | App. | App. | App. |
|---|---|---|---|---|---|
| Additive | Ex. 79 | Ex. 80 | Ex. 81 | Ex. 82 | Ex. 83 |
| Glyceryl stearate citrate | | 2.00 | 2.00 | | |
| Glyceryl stearate self-emulsifying | 5.00 | | | | |
| Stearic acid | | | | 2.50 | 3.50 |
| Stearyl alcohol | 2.00 | | | | |
| Cetyl alcohol | | | | 3.00 | 4.50 |
| Cetylstearyl alcohol | | 3.00 | 1.00 | | 0.50 |
| C₁₂-₁₅-Alkyl benzoate | | 2.00 | 3.00 | | |
| Caprylic/capric triglyceride | 2.00 | | | | |
| Octyldodecanol | 2.00 | 2.00 | | 4.00 | 6.00 |
| Dicaprylyl ether | | | | | |
| Paraffinum liquidum | | 4.00 | 2.00 | | |
| Cyclic dimethylpolysiloxane | | | | 0.50 | 2.00 |
| Dimethicone polydimethylsiloxane | 2.00 | | | | |
| Titanium dioxide | 2.00 | | | | |
| 4-Methylbenzylidenecamphor | 1.00 | | | | |
| Ethylhexyltriazone | | 3.00 | | | 2.00 |
| butyldimethoxydibenzoylmethane | 0.50 | | | | 0.50 |
| Hexyl 2-(4′-(diethylamino)-2′- | 0.50 | 1.50 | 5.00 | 3.30 | 4.00 |
| hydroxybenzoyl)benzoate | | | | | |
| Polymer Example 1 | 2.30 | 3.10 | 1.00 | 6.50 | 3.10 |
| 2-Ethylhexyl methoxycinnamate | 1.50 | 4.00 | | 2.50 | |
| 2,4-Bis(4-(2-ethylhexyloxy)-2-hydroxyl)- | 0.80 | | 1.50 | 2.50 | |
| phenyl)-6-(4-methoxyphenyl)(1,3,5)-triazine | | | | | |
| Dimethico diethylbenzalmalonate | | | 6.00 | | |
| Diethylhexylbutamidotriazone | 1.00 | 3.00 | | 2.00 | |
| Octocrylene | 4.00 | | 5.00 | | 3.50 |
| Phenylene-1,4-bis(monosodium, | 0.50 | | 1.00 | | |
| 2-benzimidazyl-5,7-disulfonic acid) | | | | | |
| Phenylbenzimidazolesulfonic acid | | 2.00 | | 1.50 | 0.50 |
| Tocopherol | | | | | 0.10 |
| Ethylenediaminetetraacetic acid trisodium | | | 0.20 | | 0.20 |
| Preservative | q.s. | q.s. | q.s. | q.s. | q.s. |
| Xanthan gum | | | 0.20 | | |
| Polyacrylic acid | 0.15 | 0.1 | | 0.05 | 0.05 |
| Sodium hydroxide solution 45% | q.s. | q.s. | q.s. | q.s. | q.s. |
| Glycerol | 3.00 | | 3.00 | 5.00 | 3.00 |
| Butylene glycol | | 3.00 | | | |
| Ethanol | | 3.00 | | 3.00 | |
| Perfume | q.s. | q.s. | q.s. | q.s. | q.s. |
| Water | ad 100 | ad 100 | ad 100 | ad 100 | ad 100 |

The analogous formulation is prepared analogously also with the copolymers from preparation examples 2-18.

### Application Examples 84-88

### Hydrodispersions

| | App. | App. | App. | App. | App. |
|---|---|---|---|---|---|
| Additive | Ex. 84 | Ex. 85 | Ex. 86 | Ex. 87 | Ex. 88 |
| Polyoxyethylene(20) cetylstearyl ether | 1.00 | | | 0.5 | |
| Cetyl alcohol | | | 1.00 | | |
| Sodium polyacrylate | | 0.20 | | 0.30 | |
| Acrylate/C₁₀-₃₀-alkyl acrylate | 0.50 | | 0.40 | 0.10 | 0.10 |
| crosspolymer | | | | | |
| Xanthan gum | | 0.30 | 0.15 | | 0.50 |
| 2-Ethylhexyl methoxycinnamate | | | | 5.00 | 8.00 |
| 2,4-Bis(4-(2-ethylhexyloxy)-2-hydroxyl)- | | 1.50 | | 2.00 | 2.50 |
| phenyl)-6-(4-methoxyphenyl)(1,3,5)- | | | | | |
| triazine | | | | | |
| Butyldimethoxydibenzoylmethane | 1.00 | | 2.00 | | |
| Diethylhexylbutamidotriazone | | 2.00 | | 2.00 | 1.00 |
| Ethylhexyltriazone | 4.00 | | 3.00 | 4.00 | |
| 4-Methylbenzylidenecamphor | 4.00 | 4.00 | | | 2.00 |
| Octocrylene | | 4.00 | 4.00 | | 2.50 |
| Phenylene-1,4-bis(monosodium, | 1.00 | | 0.50 | | 2.00 |
| 2-benzimidazyl-5,7-disulfonic acid | | | | | |
| Phenylbenzimidazolesulfonic acid | 0.50 | | | 3.00 | |
| Titanium dioxide | 0.50 | | 2.00 | 3.00 | 1.00 |
| Zinc oxide | 0.50 | 1.00 | 3.00 | | 2.00 |
| C₁₂-₁₅-Alkyl benzoate | 2.00 | 2.50 | | | |
| Dicaprylyl ether | | 4.00 | | | |
| Butylene glycol dicaprylate/dicaprate | 4.00 | | 2.00 | 6.00 | |
| Dicaprylyl carbonate | | 2.00 | 6.00 | | |
| Dimethicone polydimethylsiloxane | | 0.50 | 1.00 | | |
| Phenylmethylpolysiloxane | 2.00 | | | 0.50 | 2.00 |
| Shea butter | | 2.00 | | | |
| PVP Hexadecene copolymer | 0.50 | | | 0.50 | 1.00 |
| Octoxyglycerol | | | 1.00 | | 0.50 |
| Glycerol | 3.00 | 7.50 | | 7.50 | 2.50 |
| Glycine soya | | | 1.50 | | |
| Tocopherol acetate | 0.50 | | 0.25 | | 1.00 |
| Polymer Example 1 | 5.4 | 6.2 | 5.6 | 2.5 | 1.9 |
| Preservative | q.s. | q.s. | q.s. | q.s. | q.s. |
| Ethanol | 3.00 | 2.00 | 1.50 | | 1.00 |
| Perfume | q.s. | q.s. | q.s. | q.s. | q.s. |
| Water | ad 100 | ad 100 | ad 100 | ad 100 | ad 100 |

The analogous formulation is prepared analogously also with the copolymers from preparation examples 2-18.

### Application Examples 89-93

### Hydrodispersions

| | App. | App. | App. | App. | App. |
|---|---|---|---|---|---|
| Additive | Ex. 89 | Ex. 90 | Ex. 91 | Ex. 92 | Ex. 93 |
| Polyoxyethylene(20) cetylstearyl ether | 1.00 | | | 0.5 | |
| Cetyl alcohol | | | 1.00 | | |
| Sodium polyacrylate | | 0.20 | | 0.30 | |
| Acrylate/C₁₀-₃₀-alkyl acrylate crosspolymer | 0.50 | | 0.40 | 0.10 | 0.10 |
| Xanthan gum | | 0.30 | 0.15 | | 0.50 |
| 2-Ethylhexyl methoxycinnamate | | | | 5.00 | 8.00 |
| 2,4-Bis(4-(2-ethylhexyloxy)-2-hydroxyl)- | | 1.50 | 2.00 | | 2.50 |
| phenyl)-6-(4-methoxyphenyl)(1,3,5)- | | | | | |
| triazine | | | | | |
| Dimethico diethylbenzalmalonate | | 3.50 | | | |
| Butyldimethoxydibenzoylmethane | 1.00 | | 2.00 | | |
| Diethylhexylbutamidotriazone | | 2.00 | | 2.00 | 1.00 |
| Ethylhexyltriazone | 4.00 | | 3.00 | 4.00 | |
| 4-Methylbenzylidenecamphor | | | | | 2.00 |
| Hexyl 2-(4′-(diethylamino)-2′- | 2.00 | 1.40 | 0.50 | 4.60 | 5.20 |
| hydroxybenzoyl)benzoate | | | | | |
| Octocrylene | | 4.00 | 4.00 | | 2.50 |
| Phenylene-1,4-bis(monosodium, | 1.00 | | 0.50 | | 2.00 |
| 2-benzimidazyl-5,7-disulfonic acid) | | | | | |
| Phenylbenzimidazolesulfonic acid | 0.50 | | | 3.00 | |
| Titanium dioxide | 0.50 | | 2.00 | 3.00 | 1.00 |
| Zinc oxide | 0.50 | 1.00 | 3.00 | | 2.00 |
| C₁₂-₁₅-Alkyl benzoate | 2.00 | 2.50 | | | |
| Diethyilexyl-2,6-naphthalate | 4.00 | | | | |
| Dicaprylyl ether | | 4.00 | | | |
| Butylene glycol dicaprylat/dicaprate | 4.00 | | 2.00 | 6.00 | |
| Dicaprylyl carbonate | | 2.00 | 6.00 | | |
| Dimethicone polydimethylsiloxane | | 0.50 | 1.00 | | |
| Phenylmethylpolysiloxane | 2.00 | | | 0.50 | 2.00 |
| Shea butter | | 2.00 | | | |
| PVP Hexadecene copolymer | 0.50 | | | 0.50 | 1.00 |
| Octoxyglycerol | | | 1.00 | | 0.50 |
| Glycerol | 3.00 | 7.50 | | 7.50 | 2.50 |
| Glycine soya | | | 1.50 | | |
| Tocopherol acetate | 0.50 | | 0.25 | | 1.00 |
| Polymer Example 1 | 6.3 | 2.10 | 4.50 | 3.00 | 1.20 |
| DMDM Hydantoin | | 0.25 | | 0.60 | 0.45 |
| Parabens | 0.15 | | 0.50 | 0.30 | |
| Konkaben LMB ® | 0.10 | | 0.30 | | |
| Trisodium EDTA | | | 0.70 | | 1.00 |
| Phenoxyethanol | | 0.40 | | 0.20 | 0.50 |
| Ethanol | 3.00 | 2.00 | 1.50 | | 1.00 |
| Perfume | q.s. | q.s. | q.s. | q.s. | q.s. |
| Water | ad 100 | ad 100 | ad 100 | ad 100 | ad 100 |

The analogous formulation is prepared analogously also with the copolymers from preparation examples 2-18.

### Application Example 94

### W/O/W Cream

| Additive | % by wt. |
|---|---|
| Glyceryl stearate | 3.00 |
| PEG-100 Stearate | 0.75 |
| Behenyl alcohol | 2.00 |
| Caprylic/capric triglyceride | 8.0 |
| Octyldodecanol | 5.00 |
| C₁₂-₁₅-Alkyl benzoate | 3.00 |
| Polymer Example 1 | 5.00 |
| Ethylhexyl methoxycinnamate | 5.00 |
| Bisethylhexyloxyphenol methoxyphenyltriazine | 1.80 |
| Ethylhexyltriazone | 1.50 |
| Magnesium sulfate (MgSO₄) | 0.80 |
| Ethylendiaminetetraacetic acid | 0.10 |
| Preservative | q.s. |
| Perfume | q.s. |
| Water | ad 100.0 |

pH adjusted to 6.0

The application example is repeated but instead of the polymer from example 1, copolymers from preparation examples 2-18 are used.

### Application Examples 97-99

### Conditioner Shampoo with Pearlescence

| | App. Ex. | App. Ex. | App. Ex. |
|---|---|---|---|
| Additive | 97 | 98 | 99 |
| Polyquaternium-10 | 0.5 | 0.5 | 0.5 |
| Sodium laureth sulfate | 9.0 | 9.0 | 9.0 |
| Cocoamidopropylbetaine | 2.5 | 2.5 | 2.5 |
| Benzophenone-3 | 1.5 | 0.5 | 1.00 |
| Pearlizing agent | 2.0 | 2.0 | 2.0 |
| Polymer Example 1 | 2.1 | 3.5 | 4.05 |
| Disodium EDTA | 0.1 | 0.2 | 0.15 |
| Preservative, perfume, thickener, pH | q.s. | q.s. | q.s. |
| adjustment and solubility promoter | | | |
| Water, demin (demineralized) | ad 100.0 | ad 100.0 | ad 100.0 |

The pH is adjusted to 6.

The analogous formulation is prepared analogously also with the copolymers from preparation examples 2-18.

### Application Examples 100-103

### Conditioner Shampoo

| | App. Ex. | App. Ex. | App. Ex. |
|---|---|---|---|
| Additive | 100 | 101 | 102 |
| Polyquaternium-10 | 0.5 | 0.5 | 0.5 |
| Sodium laureth sulfate | 9.0 | 9.0 | 9.0 |
| Benzophenone 3 | 1.00 | 1.50 | 0.50 |
| Cocoamidopropylbetaine | 2.5 | 2.5 | 2.5 |
| Polymer Example 1 | 2.5 | 3.15 | 6.1 |
| Iminodisuccinic acid Na salt | 0.2 | 0.3 | 0.8 |
| Preservative, perfume, thickener, pH | q.s. | q.s. | q.s. |
| adjustment and solubility promoter | | | |
| Water, demin (demineralized) | ad 100.0 | ad 100.0 | ad 100.0 |

The pH is adjusted to 6

The analogous formulation is prepared analogously also with the copolymers from preparation examples 2-18.

### Application Examples 103-107

### Conditioner Shampoo

| | App. | App. | App. | App. | App. Ex. |
|---|---|---|---|---|---|
| Additive | Ex. 103 | Ex. 104 | Ex. 105 | Ex. 106 | 107 |
| Amphotensid GB 2009 | 10.00 | 15.00 | 20.00 | 12.00 | 17.00 |
| Plantacare 2000 | 5.00 | 6.00 | 7.00 | 8.00 | 4.00 |
| Tego Betain L7 | 15.00 | 12.00 | 10.00 | 18.00 | 20.00 |
| Luviquat FC 550 | 0.30 | 0.20 | 0.20 | 0.20 | 0.30 |
| Perfume | 0.10 | 0.10 | 0.10 | 0.10 | 0.10 |
| Polymer Example 1 | 2.00 | 4.00 | 7.00 | 1.90 | 6.00 |
| Cremophor PS 20 | 5.00 | 1.00 | 1.00 | 7.00 | 5.00 |
| Preservative | 0.10 | 0.10 | 0.10 | 0.10 | 0.10 |
| Rewopal LA 3 | 2.00 | 1.00 | 0.50 | 2.00 | 2.00 |
| Citric acid | 0.20 | 0.20 | 0.20 | 0.20 | 0.20 |
| Stepan PEG 600 DS | 3.00 | 2.00 | 2.00 | 3.00 | 2.50 |
| Water demim. | ad 100 | ad 100 | ad 100 | ad 100 | ad 100 |

The analogous formulation is prepared analogously also with the copolymers from preparation examples 2-18.

### Application Examples 108-110

### Light Shampoo with Volume Effect

| | App. Ex. | Ânbsp. | App. Ex. |
|---|---|---|---|
| Additive | 108 | 109 | 110 |
| Sodium laureth sulfate | 10.0 | 10.0 | 10.0 |
| Ethylhexyl methoxycinnamate | 2 | 2 | 2 |
| Cocoamidopropylbetaine | 2.5 | 2.5 | 2.5 |
| Polymer Example 1 | 3.05 | 1.1 | 2.01 |
| Disodium EDTA | 0.2 | 0.15 | 0.7 |
| Preservative, perfume, thickener, pH | q.s. | q.s. | q.s. |
| adjustment and solubility promoter | | | |
| Water, demin (demineralized) | ad 100.0 | ad 100.0 | ad 100.0 |

The pH is adjusted to 5.5.

The analogous formulation is prepared analogously also with the copolymers from preparation examples 2-18.

### Application Examples 111-115

### Shampoo

| | App. Ex. | App. | App. | App. | App. Ex. |
|---|---|---|---|---|---|
| Additive | 111 | Ex. 112 | Ex. 113 | Ex. 114 | 115 |
| Texapon NSO | 35.00 | 40.00 | 30.00 | 45.00 | 27.00 |
| Plantacare 2000 | 5.00 | 5.50 | 4.90 | 3.50 | 7.00 |
| Tego Betain L7 | 10.00 | 5.00 | 12.50 | 7.50 | 15.00 |
| Perfume | 0.10 | 0.10 | 0.10 | 0.10 | 0.10 |
| Polymer Example 1 | 3.50 | 3.50 | 10.5 | 10.00 | 20.00 |
| D-Panthenol USP | 0.50 | 1.00 | 0.80 | 1.50 | 0.50 |
| Preservative | 0.10 | 0.10 | 0.10 | 0.10 | 0.10 |
| Citric acid | 0.10 | 0.10 | 0.10 | 0.10 | 0.10 |
| Rewopal LA 3 | 0.50 | 2.00 | 0.50 | 0.50 | 2.00 |
| Sodium chloride | 1.50 | 1.50 | 1.50 | 1.50 | 1.50 |
| Water dem. | ad 100 | ad 100 | ad 100 | ad 100 | ad 100 |

The analogous formulation is prepared analogously also with the copolymers from preparation examples 2-18.

### Application examples 116-120

### Solids-Stabilized Emulsions

| | App. | App. | App. | App. | App. |
|---|---|---|---|---|---|
| Additive | Ex. 116 | Ex. 117 | Ex. 118 | Ex. 119 | Ex. 120 |
| Mineral oil | | | 16.0 | 16.0 | |
| Octyldodecanol | 9.0 | 9.0 | 5.0 | | |
| Caprylic/capric triglyceride | 9.0 | 9.0 | 6.0 | | |
| C₁₂-₁₅-Alkyl benzoate | | | | 5.0 | 8.0 |
| Butylene glycol | | | | | 8.0 |
| dicaprylate/dicaprate | | | | | |
| Dicaprylyl ether | 9.0 | | | 4.0 | |
| Dicaprylyl carbonate | | 9.0 | | | |
| Hydroxyoctacosanyl hydroxy- | 2.0 | 2.0 | 2.0 | 2.0 | 1.5 |
| stearate | | | | | |
| Disteardimonium hectorite | 1.0 | 0.75 | 0.5 | 0.5 | 0.25 |
| Cera microcristallina + Paraffinum | | | | | 5.0 |
| liquidum | | | | | |
| Hydroxypropylmethylcellulose | | | | | 0.05 |
| Dimethicone | | | | | 3.0 |
| Ethylhexyl methoxycinnamate | | | | | 3.0 |
| 4-Methylbenzylidenecamphor | | | | | 4.0 |
| Diethylhexylbutamidotriazone | | | | | 4.0 |
| Methylenebisbenzotriazolyl | | | | | 4.0 |
| tetramethylbutylphenol | | | | | |
| Bisethylhexyloxyphenol- | | 0.5 | | 2.00 | 1.00 |
| methoxyphenyltriazine | | | | | |
| Drometrizoletrisiloxane | | 0.50 | | 1.00 | |
| Terephthalidenedicamphorsulfonic | | 1.00 | 0.50 | | 1.50 |
| acid | | | | | |
| Phenyldibenzimidazoletetra- | | | | 1.50 | 0.5 |
| sulfonic acid | | | | | |
| Titanium dioxide + alumina + simethicone + aqua | | 2.0 | 4.0 | 2.0 | 4.0 |
| Titanium dioxide + trimethoxycaprylylsilane | | | | | 3.0 |
| Zinc oxide | | | | 6.0 | |
| Silicadimethylsilylate | | | 1.0 | | |
| Boron nitride | 2.0 | | | | |
| Starch/sodium metaphosphate | | 0.5 | | | |
| polymer | | | | | |
| Tapioca starch | | | | 1.0 | |
| Polymer Example 1 | 2.80 | 4.10 | 1.40 | 6.50 | 1.00 |
| Hexyl 2-(4′-(diethylamino)-2′- | 0.40 | 1.80 | 5.00 | 3.50 | 4.00 |
| hydroxybenzoyl)benzoate | | | | | |
| Sodium chloride | 1.0 | 1.0 | 1.0 | 1.0 | 1.0 |
| Glycerol | 5.0 | 10.0 | 3.0 | 6.0 | 10.0 |
| Trisodium EDTA | | 1.0 | | 1.0 | |
| Methyl paraben | 0.21 | | | | 0.2 |
| Propyl paraben | 0.07 | | | | |
| Phenoxyethanol | 0.5 | | 0.4 | 0.4 | 0.5 |
| Hexamidine diisethionate | | | | | 0.08 |
| Diazolidinylurea | | | 0.28 | 0.28 | |
| Alcohol | | | | 2.5 | |
| Perfume | q.s. | q.s. | q.s. | q.s. | q.s. |
| Water | ad 100 | ad 100 | ad 100 | ad 100 | ad 100 |

The analogous formulation is prepared analogously also with the copolymers from preparation examples 2-18.

### Application Examples 121-125

### Antiperspirant Roll-On

| | | | App. | App. | App. | App. | App. |
|---|---|---|---|---|---|---|---|
| | Additive | INCI | Ex. 121 | Ex. 122 | Ex. 123 | Ex. 124 | Ex. 125 |
| Phase A | Natrosol 250 HR | Hydroxyethyl- | 0.4 | 0.2 | 0.3 | 0.4 | 0.3 |
| | | cellulose | | | | | |
| | Water dem. | Water | 30 | 30 | 30 | 30 | 30 |
| Phase B | Cremophor CO | PEG-40 | 2 | 2.5 | 3 | 3.5 | 3 |
| | 40 | Hydrogenated | | | | | |
| | | Castor Oil | | | | | |
| | Bisabolol rac | Bisabolol | 0.1 | 0.1 | 0.1 | 0.1 | 0.1 |
| | Farnesol | Farnesol | 0.3 | 0.2 | 0.3 | 0.1 | 0.3 |
| | Perfume | Perfume | 0.1 | 0.2 | 0.2 | 0.1 | 0.3 |
| | Water dem. | Water | ad 100 | ad 100 | ad 100 | ad 100 | ad 100 |
| | Ethanol 96% | Alcohol | 25 | 30 | 35 | 30 | 32 |
| | Polymer Example 1 | | 2 | 5 | 7 | 5 | 6 |
| Phase C | 1,2-Propylene | Propylene Glycol | 3 | 2 | 2 | 3 | 2.5 |
| | Glycol Care | | | | | | |
| | Luviquat FC 370 | Polyquaternium- | 3 | 2.5 | 2 | 3.5 | 4 |
| | | 16 | | | | | |
| | Allantoin | Allantoin | 0.1 | 0.1 | 0.1 | 0.1 | 0.1 |
| | Locron L | Aluminum | 5 | 5.5 | 7.5 | 6 | 5.5 |
| | | Chlorohydrate | | | | | |

To produce the antiperspirant roll-on, phase A is allowed to swell; then phase B and phase C are dissolved separately. The solutions of phases B and C are stirred into phase A. The analogous formulation is prepared analogously also with the copolymers from preparation examples 2-18.

### Application Examples 126-129

### Sunscreen Gel Cream

| | | App. | App. | App. |
|---|---|---|---|---|
| Additive | App. Ex. 126 | Ex. 127 | Ex. 128 | Ex. 129 |
| Acrylate/C10-30 alkyl acrylate | 0.40 | 0.35 | 0.40 | 0.35 |
| crosspolymer | | | | |
| Polyacrylic acid | 0.20 | 0.22 | 0.20 | 0.22 |
| Xanthan Gum | 0.10 | 0.13 | 0.10 | 0.13 |
| Cetearyl alcohol | 3.00 | 2.50 | 3.00 | 2.50 |
| C12-15 Alkyl benzoate | 4.00 | 4.50 | 4.00 | 4.50 |
| Caprylic/capric triglyceride | 3.00 | 3.50 | 3.00 | 3.50 |
| Uvinul A Plus | 2.00 | 1.50 | 0.75 | 1.00 |
| UVASorb K2A | 2.0 | 3.00 | | |
| Ethylhexyl methoxycinnamate | 3.00 | | 1.00 | |
| Bisethylhexyloxyphenol | 1.5 | 1.50 | | 2.00 |
| methoxyphenyl triazine | | | | |
| Butylmethoxydibenzoylmethane | 1.0 | | 2.00 | |
| Disodium phenyl | 2.50 | | 0.50 | 2.00 |
| dibenzimidazoletetrasulfonate | | | | |
| Ethyhexyl triazone | 4.00 | | 3.00 | 4.00 |
| Octocrylene | 1.2 | 4.00 | | |
| Diethylhexylbutamidotriazone | 1.00 | | | 2.00 |
| Phenylbenzimidazolesulfonic acid | 0.50 | | 3.00 | |
| Methylenebisbenzotriazolyltetramethyl- | 2.00 | | 0.50 | 1.50 |
| butylphenol | | | | |
| Ethylhexyl salicylate | 0.3 | | 3.00 | |
| Drometrizoletrisiloxane | 0.6 | | 0.50 | |
| Terephthalidene dicamphor sulfonic acid | 0.3 | 1.50 | | 1.00 |
| Diethylhexyl 2,6-naphthalate | 4.0 | | 7.00 | |
| Microfine titanium dioxide | 6.00 | | 3.00 | |
| Microfine zinc oxide | 2.0 | 9.00 | | 5.25 |
| Polymer Example 1 | 10.30 | 5.00 | 4.00 | 8.00 |
| Cyclic dimethylpolysiloxane | 5.00 | 5.50 | 5.00 | 5.50 |
| Dimethicone polydimethylsiloxane | 1.00 | 0.60 | 1.00 | 0.60 |
| Glycerol | 1.00 | 1.20 | 1.00 | 1.20 |
| Sodium hydroxide | q.s. | q.s. | q.s. | q.s. |
| Preservative | 0.30 | 0.23 | 0.30 | 0.23 |
| Perfume | 0.20 | | 0.20 | |
| Water | ad 100 | ad 100 | ad 100 | ad 100 |

pH adjusted to 6.0

The analogous formulation is prepared analogously also with the copolymers from preparation examples 2-18.

### Application Examples 130-136

### O/W Sunscreen Formulation

| | App. | App. | App. | App. | App. | App. | App. |
|---|---|---|---|---|---|---|---|
| Additive | Ex. 130 | Ex. 131 | Ex. 132 | Ex. 133 | Ex. 134 | Ex. 135 | Ex. 136 |
| Glycerol monostearate SE | 0.50 | 1.00 | 3.00 | | | 1.50 | |
| Glyceryl stearate citrate | 2.00 | | 1.00 | 2.00 | 4.00 | | |
| Stearic acid | | 3.00 | | 2.00 | | | |
| PEG-40 Stearate | 0.50 | | | | | 2.00 | |
| Cetyl phosphate | | | | | | 1.00 | |
| Cetearyl sulfate | | | | | | | 0.75 |
| Stearyl alcohol | | | 3.00 | | | 2.00 | 0.60 |
| Cetyl alcohol | 2.50 | 1.10 | | 1.50 | 0.60 | | 2.00 |
| Polymer Example 1 | 2.00 | 5.00 | 7.00 | 10.00 | 8.00 | 5.50 | 1.00 |
| Uvinul A Plus | 2.00 | 1.50 | 0.75 | 1.00 | 2.10 | 4.50 | 5.00 |
| UVASorb K2A | 0.5 | | | | | | |
| Ethylhexyl methoxycinnamate | 2.0 | | | | 5.00 | 6.00 | 8.00 |
| Bisethylhexyloxyphenol | 0.4 | 1.50 | | 2.00 | 2.50 | | 2.50 |
| methoxyphenyltriazine | | | | | | | |
| butylmethoxydibenzoylmethane | 4.0 | | 2.00 | | 2.00 | 1.50 | |
| Disodium phenyl | 2.50 | | 0.50 | 2.00 | | 0.30 | |
| dibenzimidazoletetrasulfonate | | | | | | | |
| Ethyhexyl triazone | 4.00 | | 3.00 | 4.00 | | 2.00 | |
| Octocrylene | 2.0 | 4.00 | | | | | 7.50 |
| Diethylhexyl butamidotriazone | 1.00 | | | 2.00 | 1.00 | | 1.00 |
| Phenylbenzimidazolesulfonic | 0.50 | | 3.00 | | | | |
| acid | | | | | | | |
| methylenebisbenzotriazolyl- | 2.00 | | 0.50 | 1.50 | 2.50 | | |
| tetramethylbutylphenol | | | | | | | |
| Ethylhexyl salicylate | 0.3 | | 3.00 | | | | 5.00 |
| Drometrizoletrisiloxane | 1.0 | | 0.50 | | | 1.00 | |
| Terephthalidenedicamphor | 0.2 | 1.50 | | 1.00 | 1.00 | | 0.50 |
| sulfonic acid | | | | | | | |
| Diethylhexyl 2,6-naphthalate | 3.50 | | 7.00 | | 3.50 | 4.00 | |
| Microfine titanium dioxide | 1.00 | | 3.00 | | 3.50 | | 1.50 |
| Microfine zinc oxide | 1.0 | | | 0.25 | | 2.00 | |
| C₁₂₋₁₅-Alkyl benzoate | | 0.25 | | | 4.00 | 7.00 | |
| Dicapryl ether | | | 3.50 | | 2.00 | | |
| Butylene glycol | 5.00 | | 6.00 | | | | |
| Dicaprylate/dicaprate | | | | | | | |
| Cocoglycerides | | | 6.00 | | 2.00 | | |
| Dimethicone | 0.50 | | 1.00 | | 2.00 | | |
| Cyclomethicone | 2.00 | | 0.50 | | 0.50 | | |
| Shea butter | | 2.00 | | | | | |
| PVP Hexadecene copolymer | 0.20 | | | 0.50 | | 1.00 | |
| Glycerol | 3.00 | 7.50 | | 7.50 | 5.00 | | 2.50 |
| Xanthan gum | 0.15 | | 0.05 | | | 0.30 | |
| Sodium carbomer | | 0.20 | | 0.15 | 0.25 | | |
| Vitamin E acetate | 0.60 | | 0.23 | | 0.70 | 1.00 | |
| Glycin soya | | | | 0.50 | | 1.50 | 1.00 |
| Ethylhexyloxyglycine | 0.30 | | | | | | |
| DMDM Hydantoin | | 0.60 | 0.40 | 0.20 | | | |
| Glyacil-L | | | | 0.18 | 0.20 | | |
| Methylparaben | 0.15 | | 0.25 | | 0.50 | | |
| Phenoxyethanol | 1.00 | 0.40 | | | 0.40 | 0.50 | 0.40 |
| Trisodium EDTA | 0.02 | | 0.05 | | | | |
| Iminosuccinic acid | | | | 0.25 | 1.00 | | |
| Ethanol | 2.00 | 1.50 | | 3.00 | | 1.20 | 5.00 |
| Perfume | 0.10 | 0.25 | 0.30 | | 0.40 | 0.20 | |
| Water | ad 100 | ad 100 | ad 100 | ad 100 | ad 100 | ad 100 | ad 100 |

The analogous formulation is prepared analogously also with the copolymers from preparation examples 2-18.

### Application Examples 137-141

### Cosmetic After-Sun Formulations

| | App. | App. | App. | App. | App. |
|---|---|---|---|---|---|
| Additive | Ex. 137 | Ex. 138 | Ex. 139 | Ex. 140 | Ex. 141 |
| Ceteaereth-20 | 1.00 | | | 0.50 | |
| Cetyl alcohol | | | 1.00 | | |
| Luvigel EM | | 2.00 | | 2.50 | 2.00 |
| Acrylate/C10-30 alkyl | 0.50 | 0.30 | 0.40 | 0.10 | 0.50 |
| acrylate crosspolymer | | | | | |
| Xanthan gum | | 0.30 | 0.15 | | |
| Polymer Example 1 | 3.00 | 6.00 | 2.00 | 6.50 | 8.50 |
| C12-15 Alkyl benzoate | 2.00 | 2.50 | | | |
| Dicapryl ether | | 4.00 | | | |
| Butylene glycol | 4.00 | | 2.00 | 6.00 | |
| dicaprylate/dicaprate | | | | | |
| Dicapryl carbonate | | 2.00 | 6.00 | | |
| Dimethicone | | 0.50 | 1.00 | | |
| Phenyltrimethicone | 2.00 | | 0.50 | | |
| Tricontanyl PVP | 0.50 | | 1.00 | | |
| Ethylhexylglycerol | | | 1.00 | | 0.80 |
| Glycerol | 3.00 | 7.50 | | 7.50 | 8.50 |
| Glycine soya | | | 1.50 | | 1.00 |
| Vitamin E acetate | 0.50 | | 0.25 | | 1.00 |
| Alpha-Glucosilrutin | 0.60 | | | 0.25 | |
| Trisodium EDTA | | 0.01 | 0.05 | | 0.10 |
| Ethanol | 15.00 | 10.00 | 8.00 | 12.00 | 9.00 |
| Perfume | 0.20 | | 0.05 | 0.40 | |
| Water | ad 100 | ad 100 | ad 100 | ad 100 | ad 100 |

The analogous formulation is prepared analogously also with the copolymers from preparation examples 2-18.

Cosmetic Formulations for Decorative Cosmetics

### Application Examples 142-148

| | App. | App. | App. | App. | App. | Anbsp. | App. |
|---|---|---|---|---|---|---|---|
| Additive | Ex. 142 | Ex. 143 | Ex. 144 | Ex. 145 | Ex. 146 | 147 | Ex. 148 |
| Glycerol monostearate SE | 0.50 | 1.00 | 3.00 | | | 1.50 | |
| Glyceryl stearate citrate | 2.00 | | 1.00 | 2.00 | 4.00 | | |
| Stearic acid | | 3.00 | | 2.00 | | | |
| PEG-40 Stearate | 0.50 | | | | | 2.00 | |
| Cetyl phosphate | | | | | | 1.00 | |
| Cetearyl sulfate | | | | | | | 0.75 |
| Stearyl alcohol | | | 3.00 | | | 2.00 | 0.60 |
| Cetyl alcohol | 2.50 | 1.10 | | 1.50 | 0.60 | | 2.00 |
| Polymer Example 1 | 2.00 | 5.00 | 7.00 | 5.50 | 7.50 | 10.00 | 1.00 |
| Titanium dioxide | 10.00 | 12.00 | 9.00 | 8.50 | 11.00 | 9.50 | 10.00 |
| Iron oxides | 2.00 | 4.00 | 3.00 | 5.00 | 3.40 | 6.00 | 4.40 |
| Zinc oxide | | 4.00 | | 2.00 | | 3.00 | |
| C12-15 Alkyl benzoate | | 0.25 | | | 4.00 | 7.00 | |
| Dicapryl ether | | | 3.50 | | 2.00 | | |
| Butylene glycol | 5.00 | | 6.00 | | | | |
| dicaprylate/dicaprate | | | | | | | |
| Cocoglycerides | | | 6.00 | | 2.00 | | |
| Dimethicone | 0.50 | | 1.00 | | 2.00 | | |
| Cyclomethicone | 2.00 | | 0.50 | | 0.50 | | |
| Shea butter | | 2.00 | | | | | |
| PVP Hexadecene | 0.20 | | | 0.50 | | 1.00 | |
| copolymer | | | | | | | |
| Glycerol | 3.00 | 7.50 | | 7.50 | 5.00 | | 2.50 |
| Xanthan gum | 0.15 | | 0.05 | | | 0.30 | |
| Sodium carbomer | | 0.20 | | 0.15 | 0.25 | | |
| Vitamin E acetate | 0.60 | | 0.23 | | 0.70 | 1.00 | |
| Glycine soya | | | | 0.50 | | 1.50 | 1.00 |
| Ethylhexyloxyglycine | 0.30 | | | | | | |
| DMDM Hydantoin | | 0.60 | 0.40 | 0.20 | | | |
| Glyacil-L | | | | 0.18 | 0.20 | | |
| Methylparaben | 0.15 | | 0.25 | | 0.50 | | |
| Phenoxyethanol | 1.00 | 0.40 | | | 0.40 | 0.50 | 0.40 |
| Trisodium EDTA | 0.02 | | 0.05 | | | | |
| Iminosuccinic acid | | | | 0.25 | 1.00 | | |
| Ethanol | 2.00 | 1.50 | | 3.00 | | 1.20 | 5.00 |
| Perfume | 0.10 | 0.25 | 0.30 | | 0.40 | 0.20 | |
| Water | ad 100 | ad 100 | ad 100 | ad 100 | ad 100 | ad100 | ad 100 |

The analogous formulation is prepared analogously also with the copolymers from preparation examples 2-18.

Cleaning Formulations for Showering/Bathing/Washing

### Application Examples 149-153

| | App. Ex. | App. | App. | App. | App. Ex. |
|---|---|---|---|---|---|
| Additive | 149 | Ex. 150 | Ex. 151 | Ex. 152 | 153 |
| Texapon N 70 | 13.00 | 15.00 | 10.50 | 12.50 | 10.00 |
| Dehyton PK 45 | 7.50 | 7.00 | 5.00 | 5.50 | 10.00 |
| Cetiol HE | 2.00 | 2.50 | 3.50 | 5.00 | 2.30 |
| Perfume | 0.10 | 0.10 | 0.10 | 0.10 | 0.10 |
| Polymer Example 1 | 1.00 | 4.50 | 7.00 | 1.40 | 3.00 |
| D-Panthenol USP | 1.00 | 1.50 | 1.80 | 1.70 | 1.40 |
| Preservative | 0.10 | 0.10 | 0.10 | 0.10 | 0.10 |
| Citric acid | 0.10 | 0.10 | 0.10 | 0.10 | 0.10 |
| Luviquat Ultra Care | 1.50 | 1.00 | 1.50 | 1.20 | 1.10 |
| Sodium chloride | 1.50 | 1.40 | 1.40 | 1.30 | 1.50 |
| Water dem. | ad 100 | ad 100 | ad 100 | ad 100 | ad 100 |

The analogous formulation is prepared analogously also with the copolymers from preparation examples 2-18.

Cleaning Formulations for Showering/Bathing/Washing

### Application Examples 154-158

| | App. Ex. | App. | App. | App. | App. |
|---|---|---|---|---|---|
| Additive | 154 | Ex. 155 | Ex. 156 | Ex. 157 | Ex. 158 |
| Amphotensid GB 2009 | 10.00 | 15.00 | 20.00 | 12.00 | 17.00 |
| Plantacare 2000 | 5.00 | 6.00 | 7.00 | 8.00 | 4.00 |
| Tego Betain L7 | 15.00 | 12.00 | 10.00 | 18.00 | 20.00 |
| Luviquat FC 550 | 0.30 | 0.20 | 0.20 | 0.20 | 0.30 |
| Perfume | 0.10 | 0.10 | 0.10 | 0.10 | 0.10 |
| Polymer Example 1 | 3.00 | 6.00 | 5.50 | 4.00 | 1.50 |
| Cremophor PS 20 | 5.00 | 1.00 | 1.00 | 7.00 | 5.00 |
| Preservative | 0.10 | 0.10 | 0.10 | 0.10 | 0.10 |
| Rewopal LA 3 | 2.00 | 1.00 | 0.50 | 2.00 | 2.00 |
| Citric acid | 0.20 | 0.20 | 0.20 | 0.20 | 0.20 |
| Stepan PEG 600 DS | 3.00 | 2.00 | 2.00 | 3.00 | 2.50 |
| Water dem. | ad 100 | ad 100 | ad 100 | ad 100 | ad 100 |

The analogous formulation is prepared analogously also with the copolymers from preparation examples 2-18.

### Application Example 159

### VOC 80 Aerosol Hairspray

| Additive | % |
|---|---|
| Polymer Example 1 | 2.00 |
| Water | 18.00 |
| Dimethyl ether | 40.00 |
| Ethanol | 40.00 |

Further additive: silicone, perfume, antifoam etc.

The application example is repeated, but instead of the polymer from example 1, copolymers from preparation examples 2-18 are used.

### Application Example 160

### VOC 55 Aerosol Hairspray

| Additive | % |
|---|---|
| Polymer Example 1 | 2.00 |
| Water | 33.00 |
| Dimethyl ether | 40.00 |
| Ethanol | 25.00 |

Further additive: silicone, perfume, antifoam,

The application example is repeated, but instead of the polymer from example 1, copolymers from preparation examples 2-18 are used.

### Application Example 161

### VOC 55 Aerosol Hairspray

| Additive | % |
|---|---|
| Polymer Example 1 | 5.00 |
| Ultrahold ® Strong (BASF) | 1.00 |
| Water | 39.00 |
| Dimethyl ether | 40.00 |
| Ethanol | 15.00 |
| +AMP | to pH 8.3 |

Further additive: silicone, perfume, antifoam, etc.

The application example is repeated, but instead of the polymer from example 1, copolymers from preparation examples 2-18 are used.

### Application Example 162

| Additive | % |
|---|---|
| Polymer Example 1 | 4.00 |
| Stepanhol ® R-1^{*)} (Stepan Chemical Co.) | 1.00 |
| Water | 40.00 |
| Dimethyl ether | 40.00 |
| Ethanol | 15.00 |
| +AMP | to pH 8.3 |

Further additive: silicone, perfume, antifoam, etc.
^{*)}Stepanhold R-1 = Poly(vinylpyrrolidone/ethyl methacrylate/methacrylic acid)

### VOC 55 Aerosol Hairspray

The application example is repeated, but instead of the polymer from example 1, copolymers from preparation examples 2-18 are used.

### Application Example 163

### Liquid Make-Up

| Additive | |
|---|---|
| Phase A | |
| Glyceryl stearate | 1.70 |
| Cetyl alcohol | 1.70 |
| Ceteareth-6 | 1.70 |
| Ceteareth-25 | 1.70 |
| Caprylic/capric triglyceride | 5.20 |
| Mineral oil | 5.20 |
| Phase B | |
| Preservative | q.s. |
| Propylene glycol | 4.30 |
| Polymer Example 1 | 2.50 |
| Dist. water | 59.50 |
| Phase C | |
| Perfume oil | q.s. |
| Phase D | |
| Iron oxide | 2.00 |
| Titanium dioxide | 12.00 |

Preparation: Phase A and phase B are heated separately from one another to 80° C. Phase B is then mixed into phase A using a stirrer. Everything is left to cool to 40° C. and then phase C and phase D are added. The mixture is homogenized several times. The application example is repeated, but instead of the polymer from example 1, copolymers from preparation examples 2-18 are used.

### Application Example 164

### Face Mask

| Phase A | |
|---|---|
| Ceteareth-6 | 3.00 |
| Ceteareth-25 | 1.50 |
| Cetearyl alcohol | 5.00 |
| Cetearyl octanoate | 6.00 |
| Mineral oil | 6.00 |
| Polymer Example 1 | 4.00 |
| Bisabolol | 0.20 |
| Glyceryl stearate | 3.00 |
| Phase B | |
| Propylene glycol | 2.00 |
| Panthenol | 5.00 |
| Preservative | q.s. |
| Dist. water | 63.80 |
| Phase C | |
| Perfume | q.s. |
| Tocopheryl acetate | 0.50 |

Preparation: Phases A and B are heated separately to about 80° C. Phase B is then stirred into phase A with homogenization; following brief afterhomogenization, the mixture is left to cool to about 40° C., phase C is added and the mixture is homogenized again.

The application example is repeated, but instead of the polymer from example 1, copolymers from preparation examples 2-18 are used.

### Application Example 165

### Peeling Cream, Type O/W

| Phase A | |
|---|---|
| Ceteareth-6 | 3.00 |
| Ceteareth-25 | 1.50 |
| Glyceryl stearate | 3.00 |
| Cetearyl alcohol, sodium cetearyl sulfate | 5.00 |
| Cetearyl octanoate | 6.00 |
| Polymer Example 1 | 3.00 |
| Mineral oil | 6.00 |
| Bisabolol | 0.20 |
| Phase B | |
| Propylene glycol | 2.00 |
| Disodium EDTA | 0.10 |
| Preservative | q.s. |
| Dist. water | 59.70 |
| Phase C | |
| Tocopheryl acetate | 0.50 |
| Perfume | q.s. |
| Phase D | |
| Polyethylene | 10.00 |

Preparation: Phases A and B are heated separately to about 80° C. Phase B is then stirred into phase A and homogenized. The mixture is left to cool to about 40° C., phase C is added and the mixture is briefly homogenized again. Phase D is then stirred in.

The application example is repeated, but instead of the polymer from example 1, copolymers from preparation examples 2-18 are used.

### Application Example 166

### Shaving Foam

| | |
|---|---|
| Ceteareth-25 | 6.00 |
| Poloxamer 407 | 5.00 |
| Dist. water | 52.00 |
| Triethanolamine | 1.00 |
| Propylene glycol | 5.00 |
| Lanolin oil PEG-75 | 1.00 |
| Polymer Example 1 | 5.00 |
| Preservative | q.s. |
| Perfume | q.s. |
| Sodium laureth sulfate | 25.00 |

Preparation: All of the components are weighed together and stirred until everything has dissolved. Bottling: 90 parts of active substance and 10 parts of propane/butane mixture 25:75.

The application example is repeated, but instead of the polymer from example 1, copolymers from preparation examples 2-18 are used.

### Application Example 167

### After Shave Balsam

| Phase A | |
|---|---|
| Acrylate/C₁₀₋₃₀ alkyl acrylate copolymer | 0.25 |
| Tocopheryl acetate | 1.50 |
| Bisabolol | 0.20 |
| Caprylic/capric triglyceride | 10.00 |
| Perfume | q.s. |
| Hydrogenated castor oil PEG-40 | 1.00 |
| Phase B | |
| Panthenol | 1.00 |
| Alcohol | 15.00 |
| Glycerol | 5.00 |
| Hydroxyethylcellulose | 0.05 |
| Polymer Example 1 | 1.92 |
| Dist. water | 64.00 |
| Phase C | |
| Sodium hydroxide | 0.08 |

Preparation: The components of phase A are mixed. Then, phase B is stirred into phase A with homogenization and briefly afterhomogenized. The mixture is neutralized with phase C and homogenized again.

The application example is repeated, but instead of the polymer from example 1, copolymers from preparation examples 2-18 are used.

### Application Example 168

### Toothpaste

| Phase A | |
|---|---|
| Dist. water | 34.79 |
| Polymer example 1 | 3.00 |
| Preservative | 0.30 |
| Glycerol | 20.00 |
| Sodium monofluorophosphate | 0.76 |
| Phase B | |
| Sodium carboxymethylcellulose | 1.20 |
| Phase C | |
| Aroma oil | 0.80 |
| Saccharin | 0.06 |
| Preservative | 0.10 |
| Bisabolol | 0.05 |
| Panthenol | 1.00 |
| Tocopheryl acetate | 0.50 |
| Silicon dioxide | 2.80 |
| Sodium lauryl sulfate | 1.00 |
| Dicalcium phosphate, anhydrous | 7.90 |
| Dicalcium phosphate dihydrate | 25.29 |
| Titanium dioxide | 0.45 |

Preparation: Phase A is dissolved. Phase B is then scattered into phase A and dissolved. Phase C is added and the mixture is left under reduced pressure at room temperature for about 45 minutes.

The application example is repeated, but instead of the polymer from example 1, copolymers from preparation examples 2-18 are used.

### Application Example 169

### Prosthesis Adhesive

| Phase A | |
|---|---|
| Bisabolol | 0.20 |
| Betacarotene | 1.00 |
| Aroma oil | q.s. |
| Cetearyl octanoate | 20.00 |
| Silicon dioxide | 5.00 |
| Polymer Example 1 | 5.00 |
| Mineral oil | 33.80 |
| Phase B | |
| PVP (20% strength solution in water) | 35.00 |

Preparation: Phase A is mixed thoroughly. Phase B is then stirred into phase A. The application example is repeated, but instead of the polymer from example 1, copolymers from preparation examples 2-18 are used.

### Application Example 170

### Lip Care Cream

| | |
|---|---|
| Phase A | |
| Cetearyl octanoate | 10.00 |
| Polybutene | 5.00 |
| Phase B | |
| Carbomer | 0.10 |
| Phase C | |
| Ceteareth-6 | 2.00 |
| Ceteareth-25 | 2.00 |
| Glyceryl stearate | 2.00 |
| Cetyl alcohol | 2.00 |
| Dimethicone | 1.00 |
| Benzophenone-3 | 1.00 |
| Bisabolol | 0.20 |
| Mineral oil | 6.00 |
| Phase D | |
| Polymer Example 1 | 8.00 |
| Panthenol | 3.00 |
| Propylene glycol | 3.00 |
| Preservative | q.s. |
| Dist. water | 54.00 |
| Phase E | |
| Triethanolamine | 0.10 |
| Phase F | |
| Tocopheryl acetate | 0.50 |
| Tocopherol | 0.10 |
| Perfume | q.s. |

Preparation: Phase A is dissolved to give a clear solution. Phase B is added and homogenized. The components of phase C are added and melted at 80° C. Phase D is heated to 80° C. Phase D is added to the mixture of phases A, B and C and homogenized. The mixture is left to cool to about 40° C., phase E and phase F are added and the mixture is homogenized again.

The application example is repeated, but instead of the polymer from example 1, copolymers from preparation examples 2-18 are used.

### Application Example 171

### Roll-On Antiperspirant

| Phase A | |
|---|---|
| Hydroxyethylcellulose | 0.40 |
| Dist. water | 50.00 |
| Phase B | |
| Alcohol | 25.00 |
| Bisabclol | 0.10 |
| Farnesol | 0.30 |
| Polymer Example 1 | 6.00 |
| PEG-40 Hydrogenated castor oil | 2.00 |
| Perfume | q.s. |
| Phase C | |
| Aluminum chlorohydrate | 5.00 |
| Propylene glycol | 3.00 |
| Dimethicone copolyol | 3.00 |
| Polyquaternium-16 | 3.00 |
| Dist. water | 2.20 |

Preparation: Phase A is allowed to swell; then phases B and C are each dissolved separately. Phase A and B are stirred into phase C.

The application example is repeated, but instead of the polymer from example 1, copolymers from preparation examples 2-18 are used.

### Application Example 172

### Pump Mousse

| Phase A | |
|---|---|
| Cocotrimonium methosulfate | 2.00 |
| Perfume | q.s. |
| Phase B | |
| Dist. water | 84.30 |
| Polyquaternium-46 (10% strength aqueous solution) | 7.00 |
| Polymer Example 1 | 5.00 |
| PEG-8 | 0.50 |
| Panthenol | 1.00 |
| Preservative | q.s. |
| PEG-25 PABA (ethoxylated p-aminobenzoic acid) | 0.20 |

Preparation: The components of phase A are mixed. The components of phase B are added in succession so that a clear solution is formed.

The application example is repeated, but instead of the polymer from example 1, copolymers from preparation examples 2-18 are used.

1. A cosmetic preparation comprising an oil-in-water emulsion, where the oil-in-water emulsion comprises
a) at least one amphiphilic polymer comprising one or more hydrophobic units A and one or more hydrophilic units (B),
where the hydrophobic units A are formed from polyisobutenes modified with terminal polar groups,
b) at least one component suitable as emulsifier having an HLB value in the range from 8 to 20,
c) at least one oil and/or fat phase and
d) water.

2. The cosmetic preparation according to claim 1, where the hydrophobic units A are obtainable by functionalizing reactive polyisobutene having a number-average molecular weight Mₙ of from 150 to 50 000 g/mol.

3. The cosmetic preparation according to claim 1, where, based on the total number of the polyisobutene molecules, at least 50 mol %, of the reactive polyisobutene to be functionalized comprises terminal double bonds.

4. The cosmetic preparation according to claim 1, where one or more hydrophilic units B of the at least one amphiphilic polymer a) are formed from repeating ethylene oxide or ethylene oxide/propylene oxide units, where the fraction of propylene oxide units is at most 50% by weight.

5. The cosmetic preparation according to claim 1, where one or more hydrophilic units B correspond to the general formula II 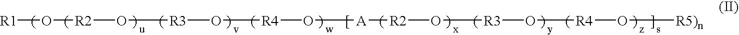
where the variables, independently of one another, have the following meanings:
R¹: hydrogen, C₁-C₂₄-alkyl, R⁶—C(═O)—, R⁶—NH—C(═O)—, polyalcohol radical;
R⁵: hydrogen, C₁-C₂₄-alkyl, R⁶—C(═O)—, R⁶—NH—C(═O)—;
R² to R⁴: —(CH₂)₂—, —(CH₂)₃—, —(CH₂)₄—, —CH₂—CH(R⁶)—, —CH₂—CHOR⁷—CH₂—;
R⁶: C₁-C₂₄-alkyl;
R⁷: hydrogen, C₁-C₂₄-alkyl, R⁶—C(═O)—, R⁶—NH—C(═O)—;
A: —C(═O)—O, —C(═O)-D-C(═O)—O, —CH₂—CH(—OH)-D-CH(—OH)—CH₂—O, —C(═O)—NH-D-NH—C(═O)O, 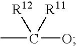
D: —(CH₂)ₜ—, arylene, optionally substituted;
R¹¹, R¹²: hydrogen, C₁—C₂₄-alkyl, C₁-C₂₄-hydroxyalkyl, benzyl or phenyl;
n: is 1 when R¹ is not a polyalcohol radical or is 1 to 500 when R¹ is a polyalcohol radical;
s=0 to 1000; t=1 to 12; u=1 to 2000; v=0 to 2000; w=0 to 2000;
x=0 to 2000; y=0 to 2000; and z=0 to 2000.

6. The cosmetic preparation according to claim 1, where the reactive polyisobutene is functionalized by a reaction which is selected from the group of reactions consisting of:
i) reaction of the reactive polyisobutene with aromatic hydroxyl compounds in the presence of an alkylation catalyst to give aromatic hydroxyl compounds alkylated with polyisobutenes,
ii) reaction of the reactive polyisobutene with a peroxy compound to give an expoxidized polyisobutene,
iii) reaction of the reactive polyisobutene with an alkene which has a double bond substituted by electron-attracting groups (enophile), in an ene reaction,
iv) reaction of the reactive polyisobutene with carbon monoxide and hydrogen in the presence of a hydroformylation catalyst to give a hydroformylated polyisobutene,
v) reaction of the reactive polyisobutene with a phosphorus halide or a phosphorus oxychloride to give a polyisobutene functionalized with phosphono groups,
vi) reaction of the reactive polyisobutene with a borane and subsequent oxidative cleavage to give a hydroxylated polyisobutene,
vii) reaction of the reactive polyisobutene with an SO₃ source to give a polyisobutene with terminal sulfo groups,
viii) reaction of the reactive polyisobutene with oxides of nitrogen and subsequent hydrogenation to give a polyisobutene with terminal amino groups, and
ix) reaction of the reactive polyisobutene with hydrogen sulfide or a thiol to give a polyisobutene functionalized with thiol groups.

7. The cosmetic preparation according to claim 1, where the amphiphilic polymers a) comprising one or more hydrophobic units A and one or more hydrophilic units (B) are obtainable by reacting functionalized polyisobutenes with alkylene oxides or by polymer-analogous reaction of functionalized polyisobutenes with polyalkylene oxides.

8. The cosmetic preparation according to claim 1, where the amphiphilic polymer a) has structures of the empirical formula AₚB_{q}, in which p and q, independently of one another, are 1 to 8.

9. The cosmetic preparation according to claim 1, where the amphiphilc polymer a) has a triblock structure ABA.

10. The cosmetic preparation according to claim 1, where the hydrophobic unit A and the hydrophilic unit (B) have a number-average molar weight Mₙ of from 150 to 50 000 g/mol.

11. The cosmetic preparation according to claim 1, where Mₙ of the hydrophobic unit A is in the range from 200 to 20 000 g/mol and Mₙ of the hydrophilic unit (B) is in the range from 500 to 30 000 g/mol.

12. The cosmetic preparation according to claim 1, where Mₙ of the hydrophobic unit A is in the range from 450 to 5000 g/mol and Mₙ of the hydrophilic unit (B) is in the range from 800 to 15 000 g/mol.

13. The cosmetic preparation according to claim 1 comprising, as amphiphilic polymer a), at least one triblock copolymer of the structure ABA constructed from polyisobutene functionalized with succinic anhydride groups (PIBSA) as hydrophobic unit A and of polyethylene oxide as hydrophilic unit (B).

14. The cosmetic preparation according to claim 1, where the cosmetic preparation is selected from the group consisting of creams, foams, sprays, gels, gel sprays, lotions, oils, oil gels and mousses.

15. The cosmetic preparation according to claim 1, where, based on the total number of the polyisobutene molecules, at least 60 mol %, of the reactive polyisobutene to be functionalized comprises terminal double bonds.

16. The cosmetic preparation according to claim 6, where the SO₃ source is acetyl sulfate or oleum.

17. The cosmetic preparation according to claim 2, where, based on the total number of the polyisobutene molecules, at least 50 mol %, of the reactive polyisobutene to be functionalized comprises terminal double bonds.

18. The cosmetic preparation according to claim 2, where one or more hydrophilic units B of the at least one amphiphilic polymer a) are formed from repeating ethylene oxide or ethylene oxide/propylene oxide units, where the fraction of propylene oxide units is at most 50% by weight.

19. The cosmetic preparation according to claim 2, where one or more hydrophilic units B correspond to the general formula II 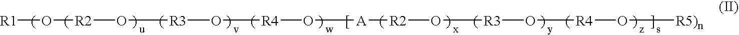
where the variables, independently of one another, have the following meanings:
R¹: hydrogen, C₁-C₂₄-alkyl, R⁶—C(═O)—, R⁶—NH—C(═O)—, polyalcohol radical;
R⁵: hydrogen, C₁-C₂₄-alkyl, R⁶—C(═O)—, R⁶—NH—C(═O)—;
R² to R⁴: —(CH₂)₂—, —(CH₂)₃—, —(CH₂)₄—, —CH₂—CH(R⁶)—, —CH₂—CHOR⁷—CH₂—;
R⁶: C₁-C₂₄-alkyl;
R⁷: hydrogen, C₁-C₂₄-alkyl, R⁶—C(═O)—, R⁶—NH—C(═O)—;
A: —C(═O)—O, —C(═O)-D-C(═O)—O, —CH₂—CH(—OH)-D-CH(—OH)—CH₂—O, —C(═O)—NH-D-NH—C(═O)—O, 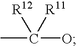
D: —(CH₂)ₜ—, arylene, optionally substituted;
R¹¹, R¹²: hydrogen, C₁-C₂₄-alkyl, C₁-C₂₄-hydroxyalkyl, benzyl or phenyl;
n: is 1 when R¹ is not a polyalcohol radical or is 1 to 500 when R¹ is a polyalcohol radical
s=0 to 1000; t=1 to 12; u=1 to 2000; v=0 to 2000; w=0 to 2000;
x=0 to 2000; y=0 to 2000; and z=0 to 2000.

20. The cosmetic preparation according to claim 2, where the reactive polyisobutene is functionalized by a reaction which is selected from the group of reactions consisting of:
i) reaction of the reactive polyisobutene with aromatic hydroxyl compounds in the presence of an alkylation catalyst to give aromatic hydroxyl compounds alkylated with polyisobutenes,
ii) reaction of the reactive polyisobutene with a peroxy compound to give an expoxidized polyisobutene,
iii) reaction of the reactive polyisobutene with an alkene which has a double bond substituted by electron-attracting groups (enophile), in an ene reaction,
iv) reaction of the reactive polyisobutene with carbon monoxide and hydrogen in the presence of hydroformylation catalyst to give a hydroformylated polyisobutene,
v) reaction of the reactive polyisobutene with a phosphorus halide or a phosphorus oxychloride to give a polyisobutene functionalized with phosphono groups,
vi) reaction of the reactive polyisobutene with a borane and subsequent oxidative cleavage to give a hydroxylated polyisobutene,
vii) reaction of the reactive polyisobutene with an SO₃ source to give a polyisobutene with terminal sulfo groups,
viii) reaction of the reactive polyisobutene with oxides of nitrogen and subsequent hydrogenation to give a polyisobutene with terminal amino groups, and
ix) reaction of the reactive polyisobutene with hydrogen sulfide or a thiol to give a polyisobutene functionalized with thiol groups.
